(19) United States
(12) Patent Application Publication
(10) Pub. No.: US 2021/0139507 A1
(43) Pub. Date: May 13, 2021

(54) FUSED PYRIDINES WHICH ACT AS INHIBITORS OF H PGDS

(71) Applicant: GlaxoSmithKline Intellectual Property Development Limited, Brentford, Middlesex (GB)

(72) Inventors: David Norman DEATON, Collegeville, PA (US); Rodolfo CADILLA, Research Triangle Park, NC (US)

(21) Appl. No.: 16/771,745

(22) PCT Filed: Dec. 12, 2018
(86) PCT No.: PCT/IB2018/059934

§ 371 Date: Jun. 11, 2020

Related U.S. Application Data
(60) Provisional application No. 62/598,003, filed on Dec. 13, 2017.

(51) Int. Cl. C07D 513/04; C07D 495/04; A61P 21/00

(52) U.S. Cl. CPC C07D 513/04; A61P 21/00; C07D 495/04

(57) ABSTRACT

A compound of formula (I)

[chemical structure]

wherein R¹, R², R³, R⁴, X, Y, and A are as defined herein.

The compounds of the present invention are inhibitors of hematopoietic prostaglandin D synthase (H-PGDS) and can be useful in the treatment of Duchenne muscular dystrophy. Accordingly, the invention is further directed to pharmaceutical compositions comprising a compound of the invention. The invention is still further directed to methods of inhibiting H-PGDS activity and treatment of disorders associated therewith using a compound of the invention or a pharmaceutical composition comprising a compound of the invention.

### FIELD OF THE INVENTION

The present invention relates to substituted thieno[3,2-b]pyridine-6-carboxamide compounds, and substituted thiazolo[4,5-b]pyridine-6-carboxamide compounds, to the use of the compounds as Hematopoietic Prostaglandin D Synthase (H-PGDS) inhibitors, to pharmaceutical compositions comprising the compounds and to the use of the compounds in therapy, especially in the treatment of conditions for which a H-PGDS inhibitor is indicated, such as asthma, neurodegenerative diseases and musculoskeletal diseases including Duchenne Muscular Dystrophy, where PGD₂ is considered to play a pathological role, for the use of a compound in the manufacture of a medicament for the treatment of conditions in which an inhibitor of H-PGDS is indicated, and a method for the treatment or prophylaxis of disorders in which inhibition of H-PGDS is indicated, in a human.

### BACKGROUND OF THE INVENTION

Prostaglandin D₂ (PGD₂) is a product of arachidonic acid metabolism, and is the major prostanoid mediator synthesised by mast cells in response to stimulation via multiple mechanisms and cellular activation pathways, including allergen-mediated cross-linking of high affinity IgE receptors (Lewis et al. (1982) Prostaglandin D₂ generation after activation of rat and human mast cells with anti-IgE. J. Immunol., 129, 1627-1631). Other cells such as dendritic cells, Th2 cells, and epithelial cells also produce PGD₂, but at lower levels than mast cells. PGD₂ mediates its effects via activation of the specific G-protein coupled receptors DP₁ (Boie et al. (1995) Molecular cloning and characterization of the human prostanoid DP receptor. (J. Biol. Chem., 270, 18910-18916) and DP₂ (CRTH2) (Abe et al. (1999), Molecular cloning, chromosome mapping and characterization of the mouse CRTH2 gene, a putative member of the leukocyte chemo-attractant receptor family. (Gene, 227, 71-77) and also acts via the receptor for thromboxane A₂ (TXA₂), the TP receptor, on target cells.

Prostaglandin D synthase (PGDS) is the enzyme responsible for the catalytic isomerase conversion of prostaglandin endoperoxide PGH₂ to PGD₂. PGD₂ is generated by the action of either H-PGDS (hematopoietic-type or H-type) or L-PGDS (lipocalin-type or L-type) enzymes (Urade et al., (2000) Prostaglandin D synthase structure and function. Vitamins and hormones, 58, 89-120). H-PGDS activity is dependent on glutathione and plays an important role in the generation of PGD₂ by immune and inflammatory cells, including mast cells, antigen-presenting cells (e.g. dendritic cells), macrophages, and Th2 cells, which are all key cells in the pathology of allergic disease. In contrast, L-type is glutathione-independent and is primarily located in the central nervous system, genital organs, and heart. These two isoforms of PGDS appear to have distinct catalytic properties, tertiary structure, and cellular and tissue distribution.

Using the small molecule inhibitor HQL-79, H-PGDS has been demonstrated to play a modulatory role in diseases such as Duchenne muscular dystrophy (Nakagawa et al. (2013) A prostaglandin D₂ metabolite is elevated in the urine of Duchenne muscular dystrophy patients and increases further from 8 years old, Clinica Chimica Acta 423, 10-14) and (Mohri et al. (2009), Inhibition of prostaglandin D synthase suppresses muscular necrosis, Am. J. Pathol. 174, 1735-1744) and (Okinaga et al. (2002), Induction of hematopoietic prostaglandin D synthase in hyalinated necrotic muscle fibers: its implication in grouped necrosis, Acta Neuropathologica 104, 377-84), spinal cord contusion injury (Redensek et al. (2011) Expression and detrimental role of hematopoietic prostaglandin D synthase in spinal cord contusion injury, Glia 59, 603-614), neuroinflammation (Mohri et al. (2006) Prostaglandin D₂-mediated microglia/astrocyte interaction enhances astrogliosis and demyelination in twitcher. J. Neurosci. 26,4383-4393), and neurodegenerative disease (Ikuko et al. (2007) Hematopoietic prostaglandin D synthase and DP₁ receptor are selectively upregulated in microglia and astrocytes within senile plaques from human patients and in a mouse model of Alzheimer disease. J. Neuropath. Exp. Neur. 66, 469-480). H-PGDS has also been implicated to play a role in metabolic diseases such as diabetes and obesity, since PGD₂ is converted to 15-deoxy-Δ^{12,14}PGJ₂, a potent ligand for PPARγ which is able to drive adipogenesis (Tanaka et al (2011) Mast cells function as an alternative modulator of adipogenesis through 15-deoxy-delta-12,14-prostaglandin J₂. Am. J. Physiol. Cell Physiol. 301, C1360-C1367). PGD₂ has been implicated to play a role in niacin-induced skin flushing (Papaliodis et al (2008) Niacin-induced “flush” involves release of prostaglandin D₂ from mast cells and serotonin from platelets: Evidence from human cells in vitro and an animal model. JPET 327:665-672).

Weber et al. (2010), Identification and characterisation of new inhibitors for the human hematopoietic prostaglandin D₂ synthase. Eur. J. Med. Chem. 45, 447-454, Carron et al. (2010), Discovery of an Oral Potent Selective Inhibitor of Hematopoietic Prostaglandin D Synthase (H-PGDS). ACS Med. Chem. Lett. 1, 59-63; Christ et al. (2010), Development and Characterization of New Inhibitors of the Human and Mouse Hematopoietic Prostaglandin D₂ Synthases, J. Med. Chem., 53, 5536-5548; and Hohwy et al. (2008), Novel Prostaglandin D Synthase Inhibitors Generated by Fragment-Based Drug Design. J. Med. Chem., 51, 2178-2186 are also of interest.

Based on this evidence, chemical inhibitors of H-PGDS which inhibit PGD₂ formation, simultaneously inhibit the biological actions of PGD₂ and its metabolites at multiple receptors and offer the potential for therapeutic benefit in the treatment of a range of diseases where PGD₂ is considered to play a pathological role.

International Patent Applications WO2005/094805, WO2007/007778, WO2007/041634, 2008/121670, WO2008/122787, WO2009/153720, WO2009/153721, WO2010/033977, WO2011/043359, WO2011044307, WO2011/090062, Japanese Patent Application 2007-51121 and US Patent Application 2008/0146569 disclose certain H-PGDS inhibitors and their use in the treatment of diseases associated with the activity of H-PGDS.

It is an object of the invention to provide further H-PGDS inhibitors, suitably for the treatment of Muscular Dystrophy.

### SUMMARY OF THE INVENTION

The invention is directed to compounds according to Formula (I):

[chemical structure]

wherein R¹, R², R³, R⁴, X, Y, and A are as defined below.

Compounds of Formula (I) and their pharmaceutically acceptable salts have H-PGDS activity and are believed to be of use for the treatment or prophylaxis of certain disorders.

Accordingly, in another aspect of the invention there is provided a pharmaceutical composition comprising a compound of Formula (I) according to the first aspect, or a pharmaceutically acceptable salt thereof and one or more pharmaceutically acceptable carriers or excipients.

In some embodiments, the pharmaceutical composition is for the treatment or prophylaxis of a disorder in which inhibition of H-PGDS is beneficial.

In a further aspect, the invention provides a compound of Formula (I) or a pharmaceutically acceptable salt thereof according to the first aspect of the invention for use in therapy.

The invention also provides a compound of Formula (I) or a pharmaceutically acceptable salt thereof, for use in the treatment of a condition for which an H-PGDS inhibitor is indicated.

This invention also relates to a method of treating Duchenne muscular dystrophy, which comprises administering to a subject in need thereof an effective amount of a H-PGDS inhibiting compound of Formula (I).

This invention also relates to a method of treating congenital myotonia, which comprises administering to a subject in need thereof an effective amount of a H-PGDS inhibiting compound of Formula (I).

This invention also relates to a method of treating muscle injury, which comprises administering to a subject in need thereof an effective amount of a H-PGDS inhibiting compound of Formula (I).

This invention also relates to a method of treating tendon injury, which comprises administering to a subject in need thereof an effective amount of a H-PGDS inhibiting compound of Formula (I).

This invention also relates to a method of treating muscle lacerations, which comprises administering to a subject in need thereof an effective amount of a H-PGDS inhibiting compound of Formula (I).

This invention also relates to a method of treating chronic muscle strains, which comprises administering to a subject in need thereof an effective amount of a H-PGDS inhibiting compound of Formula (I).

This invention also relates to a method of treating myotonic dystrophy type I, which comprises administering to a subject in need thereof an effective amount of a H-PGDS inhibiting compound of Formula (I).

This invention also relates to a method of treating myotonic dystrophy type II, which comprises administering to a subject in need thereof an effective amount of a H-PGDS inhibiting compound of Formula (I).

This invention also relates to a method of treating asthma, which comprises administering to a subject in need thereof an effective amount of a H-PGDS inhibiting compound of Formula (I).

This invention also relates to a method of treating chronic obstructive pulmonary disease, which comprises administering to a subject in need thereof an effective amount of a H-PGDS inhibiting compound of Formula (I).

This invention also relates to a method of treating rheumatoid arthritis, which comprises administering to a subject in need thereof an effective amount of a H-PGDS inhibiting compound of Formula (I).

This invention also relates to a method of treating inflammatory bowel disease, which comprises administering to a subject in need thereof an effective amount of a H-PGDS inhibiting compound of Formula (I).

This invention also relates to a method of treating osteoarthritis, which comprises administering to a subject in need thereof an effective amount of a H-PGDS inhibiting compound of Formula (I).

This invention also relates to a method of treating psoriasis, which comprises administering to a subject in need thereof an effective amount of a H-PGDS inhibiting compound of Formula (I).

This invention also relates to a method of treating atopic dermatitis, which comprises administering to a subject in need thereof an effective amount of a H-PGDS inhibiting compound of Formula (I).

This invention also relates to a method of treating a muscle degenerative disorder, which comprises administering to a subject in need thereof an effective amount of a H-PGDS inhibiting compound of Formula (I).

This invention also relates to a method of treating muscular dystrophy, which comprises administering to a subject in need thereof an effective amount of a H-PGDS inhibiting compound of Formula (I).

This invention also relates to a method of treating obesity, which comprises administering to a subject in need thereof an effective amount of a H-PGDS inhibiting compound of Formula (I).

Also included in the present invention are methods of co-administering the presently invented H-PGDS inhibiting compounds with further active ingredients.

The invention also relates to a compound of Formula (I) or a pharmaceutically acceptable salt thereof for use in the treatment of Duchenne muscular dystrophy.

The invention also relates to a compound of Formula (I) or a pharmaceutically acceptable salt thereof for use in the treatment of congenital myotonia.

The invention also relates to a compound of Formula (I) or a pharmaceutically acceptable salt thereof for use in the treatment of muscle injury.

The invention also relates to a compound of Formula (I) or a pharmaceutically acceptable salt thereof for use in the treatment of tendon injury.

The invention also relates to a compound of Formula (I) or a pharmaceutically acceptable salt thereof for use in the treatment of muscle lacerations.

The invention also relates to a compound of Formula (I) or a pharmaceutically acceptable salt thereof for use in the treatment of chronic muscle strains.

The invention also relates to a compound of Formula (I) or a pharmaceutically acceptable salt thereof for use in the treatment of myotonic dystrophy type I.

The invention also relates to a compound of Formula (I) or a pharmaceutically acceptable salt thereof for use in the treatment of myotonic dystrophy type II.

The invention also relates to a compound of Formula (I) or a pharmaceutically acceptable salt thereof for use in the treatment of asthma.

The invention also relates to a compound of Formula (I) or a pharmaceutically acceptable salt thereof for use in the treatment of chronic obstructive pulmonary disease.

The invention also relates to a compound of Formula (I) or a pharmaceutically acceptable salt thereof for use in the treatment of rheumatoid arthritis.

The invention also relates to a compound of Formula (I) or a pharmaceutically acceptable salt thereof for use in the treatment of inflammatory bowel disease.

The invention also relates to a compound of Formula (I) or a pharmaceutically acceptable salt thereof for use in the treatment of osteoarthritis.

The invention also relates to a compound of Formula (I) or a pharmaceutically acceptable salt thereof for use in the treatment of psoriasis.

The invention also relates to a compound of Formula (I) or a pharmaceutically acceptable salt thereof for use in the treatment of atopic dermatitis.

The invention also relates to a compound of Formula (I) or a pharmaceutically acceptable salt thereof for use in the treatment of a muscle degenerative disorder.

The invention also relates to a compound of Formula (I) or a pharmaceutically acceptable salt thereof for use in the treatment of muscular dystrophy.

The invention also relates to a compound of Formula (I) or a pharmaceutically acceptable salt thereof for use in the treatment of obesity.

The invention provides for the use of a compound of Formula (I) or a pharmaceutically acceptable salt thereof in the manufacture of a medicament for the treatment of conditions in which an inhibitor of H-PGDS is indicated.

The invention further provides a method for the treatment or prophylaxis of disorders in which inhibition of H-PGDS is indicated, in a human, which comprises administering a human in need thereof a therapeutically effective amount of a compound of Formula (I) or a pharmaceutically acceptable salt thereof.

### BRIEF DESCRIPTION OF THE FIGURE

FIG. 1. depicts the protection and acceleration of functional repair dose response curves of H-PGDS inhibition using the compound of Example 8 following limb muscle injury in male C57BI/6N mice.

### DETAILED DESCRIPTION OF THE INVENTION

This invention relates to novel compounds of Formula (I):

[chemical structure]

wherein:

X is absent or selected from: N, S, and O;

Y is selected from: CH, and N;

R³ is absent or selected from:
H,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl, and
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN;

R⁴ is selected from:
F,
Cl,
Br,
I,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl,
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN,
heterocycloalkyl, and
heterocycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN;

A is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S; and

R¹ and R² are independently selected from:
hydrogen,
—OS(O)₂NH₂,
—S(O)₂CH₃,
—OH,
—CN,
F,
tetrazolyl,
methyltetrazolyl,
cycloalkyl,
morpholinyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cycloalkyl, —S(O)₂CH₃, —S(O)₂NH₂, and —S(O)₂N(H)C₁-4alkyl, —NH₂,
   —N(H)C₁₋₄alkyl, —N(H)C₁₋₄alkyl where alkyl is substituted with from 1 to fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently selected from: —OH, oxo, fluoro, C₁₋₄alkoxy, cycloalkyl, —NH₂, —N(H)C₁₋₄alkyl, —N(H)C₁₋₄alkyl where the alkyl is substituted with from 1 to 5 fluoro, —N(C₁₋₄alkyl)₂, —N(C₁₋₄alkyl)₂ where the alkyls are
independently substituted with from 1 to 7 fluoro, —S(O)₂CH₃, —S(O)₂NH₂, and —S(O)₂N(H)C₁₋₄alkyl,
N(C₁₋₆alkyl)₂, where each alkyl is optionally substituted with from one to six substituents independently selected from: —OH, oxo, fluoro, and —S(O)₂CH₃,
N(H)C₁₋₆alkyl, and
N(H)C₁₋₆alkyl substituted with from one to six substituents independently selected from: —OH, oxo, fluoro, and —S(O)₂CH3;

provided R³ is absent when X is absent; and

provided R⁴ is not F, Cl, Br, or I when X is N or O;

and salts thereof.

This invention also relates to pharmaceutically acceptable salts of the compounds of Formula (I).

Suitably in the compounds of Formula (I), X is absent. Suitably in the compounds of Formula (I), X is N. Suitably in the compounds of Formula (I), X is S. Suitably in the compounds of Formula (I), X is O.

Suitably in the compounds of Formula (I), Y is CH. Suitably in the compounds of Formula (I), Y is N.

Suitably in the compounds of Formula (I), R³ is absent or selected from:
H,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl, and
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN.
   Suitably in the compounds of Formula (I), R⁴ is selected from:
F,
Cl,
Br,
I,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl,
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN,
heterocycloalkyl, and
heterocycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN.

Suitably in the compounds of Formula (I), A is selected from:
C₄₋₇cycloalkyl,
a 4-, 5-, or 6-membered heterocycloalkyl containing one or two heteroatoms independently selected from O and N,
and
a 5-12 membered heteroaryl containing one or two heteroatoms, wherein at least one heteroatom is nitrogen and the second heteroatom, if present, is selected from N and S.

Suitably in the compounds of Formula (I), R¹ and R² are independently selected from:
hydrogen,
13 OS(O)₂NH₂,
—S(O)₂CH₃,
—OH,
—CN,
F,
tetrazolyl,
methyltetrazolyl,
cycloalkyl,
morpholinyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cycloalkyl, —S(O)₂CH₃, —S(O)₂NH₂, and —S(O)₂N(H)C₁₋₄alkyl, —NH₂, —N(H)C₁₋₄alkyl, —N(H)C₁₋₄alkyl where alkyl is substituted with from 1 to 5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently selected from: —OH, oxo, fluoro, C₁₋₄alkoxy, cycloalkyl, —NH₂, —N(H)C₁₋₄alkyl, —N(H)C₁₋₄alkyl where the alkyl is substituted with from 1 to 5 fluoro, —N(C₁₋₄alkyl)₂, —N(C₁-4alkyl)₂ where the alkyls are independently substituted with from 1 to 7 fluoro, —S(O)₂CH₃, —S(O)₂NH₂, and —S(O)₂N(H)C₁₋₄alkyl,
N(C₁₋₆alkyl)₂, where each alkyl is optionally substituted with from one to six substituents independently selected from: —OH, oxo, fluoro, and —S(O)₂CH₃,
N(H)C₁₋₆alkyl, and
N(H)C₁₋₆alkyl substituted with from one to six substituents independently selected from: —OH, oxo, fluoro, and —S(O)₂OH₃.

Suitably in the compounds of Formula (I), the moiety —XR³R⁴ is selected from: bromide, cyclopropyl, methylcyclopropyl, cyclobutyl, azetidinyl, methylazetidinyl, —NHCH(CH₃)₂, —N(CH₃)CH(CH₃)₂, —NHCH₃, —N(CH₃)₂, —CF(CH₃)₂, —C(CH₃)₃, —CH(CH₃)₂, pyrrolidinyl, —N(CH₃)cyclopropyl, —N(cyclopropyl)₂, —NCH(CH₃)₂CH(CH₃)₂, —N(CH₃)C(CH₃)₃, —SCH₃, and —OCH₃.

Suitably in the compounds of Formula (I), A is selected from: cyclohexyl, cyclobutyl, bicyclopentanyl, spiroheptanyl, pyrrolidinyl, tetrahydropyranyl, and piperidinyl.

Suitably in the compounds of Formula (I), R¹ and R² are independently selected from: hydrogen, fluoro, —OH, —CH₃, —OCH₂CH₂OH, oxo, —CH₂OH, —C(CH₃)₂OH, —NHCH(CH₃)CHF₂, —CH(cyclopropyl)OH, —CH(OH)CH₂S(O)₂CH₃, tetrazolyl, methyltetrazolyl, difluoroazetidinyl, fluoroazetidinyl, azetidinyl and —CH(OH)CF₃.

Included in the presently invented compounds of Formula (I) are compounds of Formula (II):

[chemical structure]

wherein:

X¹ is absent or selected from: N, S, and O;

Y¹ is selected from: CH, and N;

R¹³ is absent or selected from:
H,
C₁₋₃alkyl,
C₁₋₃alkyl substituted with from 1 to 3 substituents independently selected from: fluoro, oxo, C₁₋₄alkoxy, —OH, and —COOH,
C₃₋₇cycloalkyl, and
C₃₋₇cycloalkyl substituted with 1 or 2 substituents independently selected from: fluoro, oxo, C₁₋₄alkoxy, —OH, —COOH, and C₁-3alkyl;

R¹⁴ is selected from:
F,
Cl,
Br,
I,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl,
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN,
heterocycloalkyl, and
heterocycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN;

A¹ is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S;

R¹¹ and R¹² are independently selected from:
H,
—OS(O)₂NH₂,
—S(O)₂CH₃,
—OH,
—CN,
F,
tetrazolyl,
methyltetrazolyl,
cyclopropyl,
morpholinyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cyclopropyl, cyclopentyl, cyclobutyl, —S(O)₂CH₃, —S(O)₂NH₂, —S(O)₂N(H)C₁₋₄alkyl, —NH₂, —N(H)C₁₋₄alkyl, —N(H)C₁₋₄alkyl where alkyl is substituted with from 1 to 5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently selected from: —OH, oxo, fluoro, C₁₋₄alkoxy, cycloalkyl, —NH₂, —N(H)C₁₋₄alkyl, —N(H)C₁₋₄alkyl where the alkyl is substituted with from 1 to 5 fluoro, —N(C₁₋₄alkyl)₂, —N(C₁₋₄alkyl)₂ where the alkyls are independently substituted with from 1 to 7 fluoro, —S(O)₂CH₃, —S(O)₂NH₂, and —S(O)₂N(H)C₁₋₄alkyl,
N(H)C₁₋₆alkyl, and
N(H)C₁₋₆alkyl substituted with from one to six substituents independently selected from: —OH, oxo, fluoro, and —S(O)₂CH₃;

provided R¹³ is absent when X¹ is absent; and

provided R¹⁴ is not F, Cl, Br, or I when X¹ is N or O;

and salts thereof.

This invention also relates to pharmaceutically acceptable salts of the compounds of Formula (II).

Suitably in the compounds of Formula (II), X¹ is absent. Suitably in the compounds of Formula (II), X¹ is N. Suitably in the compounds of Formula (II), X¹ is S. Suitably in the compounds of Formula (II), X¹ is O.

Suitably in the compounds of Formula (II), Y¹ is CH. Suitably in the compounds of Formula (II), Y¹ is N.

Suitably in the compounds of Formula (II), R¹³ is absent or selected from:
H,
C₁₋₃alkyl,
C₁₋₃alkyl substituted with from 1 to 3 substituents independently selected from: fluoro, oxo, C₁₋₄alkoxy, —OH, and —COOH,
C₃₋₇cycloalkyl, and
C₃₋₇cycloalkyl substituted with 1 or 2 substituents independently selected from: fluoro, oxo, C₁₋₄alkoxy, —OH, —COOH, and C₁-3alkyl.

Suitably in the compounds of Formula (II), R¹⁴ is selected from:
F,
Cl,
Br,
I,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl,
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN,
heterocycloalkyl, and
heterocycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN.

Suitably in the compounds of Formula (II), A¹ is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S

Suitably in the compounds of Formula (II), R¹¹ and R¹² are independently selected from:
H,
OS(O)₂NH₂,
—S(O)₂CH₃,
—OH,
—CN,
F,
tetrazolyl,
methyltetrazolyl,
cyclopropyl,
morpholinyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cyclopropyl, cyclopentyl, cyclobutyl, —S(O)₂CH₃, —S(O)₂NH₂, —S(O)₂N(H)C₁₋₄alkyl, —NH₂, —N(H)C₁₋₄alkyl, —N(H)C₁₋₄alkyl where alkyl is substituted with from 1 to 5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently selected from: —OH, oxo, fluoro, C₁₋₄alkoxy, cycloalkyl, —NH₂, —N(H)C₁₋₄alkyl, —N(H)C₁₋₄alkyl where the alkyl is substituted with from 1 to 5 fluoro, —N(C₁₋₄alkyl)₂, —N(C₁₋₄alkyl)₂ where the alkyls are independently substituted with from 1 to 7 fluoro, —S(O)₂CH₃, —S(O)₂NH₂, and —S(O)₂N(H)C₁₋₄alkyl,
N(H)C₁₋₆alkyl, and
N(H)C₁₋₆alkyl substituted with from one to six substituents independently selected from: —OH, oxo, fluoro, and —S(O)₂CH₃.

Suitably in the compounds of Formula (II), the moiety —X¹R¹³R¹⁴ is selected from: bromo, cyclopropyl, methylcyclopropyl, cyclobutyl, azetidinyl, methylazetidinyl, —NHCH(CH₃)₂, —N(CH₃)CH(CH₃)₂, —NHCH₃, —N(CH₃)₂, —CF(CH₃)₂, —C(CH₃)₃, —CH(CH₃)₂, pyrrolidinyl, —N(CH₃)cyclopropyl, —N(cyclopropyl)₂, —NCH(CH₃)₂CH(CH₃)₂, —N(CH₃)C(CH₃)₃, —SCH₃, and —OCH₃.

Suitably in the compounds of Formula (II), A¹ is selected from: cyclohexyl, cyclobutyl, bicyclopentanyl, spiroheptanyl, pyrrolidinyl, tetrahydropyranyl, and piperidinyl.

Suitably in the compounds of Formula (II), R¹¹ and R¹² are independently selected from: hydrogen, fluoro, —OH, —CH₃, —OCH₂CH₂OH, oxo, —CH₂OH, —C(CH₃)₂OH, —NHCH(CH₃)CHF₂, —CH(cyclopropyl)OH, —CH(OH)CH₂S(O)₂CH₃, tetrazolyl, methytetrazolyl, difluoroazetidinyl, fluoroazetidinyl, azetidinyl and —CH(OH)CF₃.

Included in the presently invented compounds of Formula (I) are compounds of Formula (III):

[chemical structure]

wherein:

X² is absent or selected from: N, S, and O;

Y² is selected from: CH, and N;

R²³ is absent or selected from:
H,
CH₃,
—CH₂CH₃,
CH(CH₃)₂, and
cyclopropyl;

R²⁴ is selected from:
Cl,
Br,
I,
C₁₋₄alkyl,
C₁₋₄alkyl substituted from 1 to 3 times by F,
cyclopropyl;
methylcyclopropyl,
cyclo butyl,
azetidinyl,
methylazetidinyl, and
pyrrolidinyl;

A² is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S; and

R²¹ and R²² are independently selected from:
H,
—OS(O)₂NH₂,
—S(O)₂CH₃,
—OH,
—CN,
F,
tetrazolyl
methyltetrazolyl,
cyclopropyl,
morpholinyl,
tetrazolyl,
methyltetrazolyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cyclopropyl, cyclopentyl, —S(O)₂CH₃, —NH₂, —N(H)C₁₋₄alkyl, —N(H)C₁₋₄alkyl where alkyl is substituted with from 1 to 5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently selected from: —OH, oxo, fluoro, C₁₋₄alkoxy, cyclopropyl, —NH₂, —N(H)C₁₋₄alkyl, —N(H)C₁₋₄alkyl where the alkyl is substituted with from 1 to 5 fluoro, —N(C₁₋₄alkyl)₂, —N(C₁₋₄alkyl)₂ where the alkyls are independently substituted with from 1 to 7 fluoro, —S(O)₂CH₃, —S(O)₂NH₂, and —S(O)₂N(H)C₁₋₄alkyl,
N(H)C₁₋₆alkyl, and
N(H)C₁₋₆alkyl substituted with from one to six substituents independently selected from: —OH, oxo, fluoro, and —S(O)₂CH₃;
provided R²³ is absent when X² is absent; and
provided R is not Cl, Br, or I when X is N or O;
   and salts thereof.

This invention also relates to pharmaceutically acceptable salts of the compounds of Formula (III).

Suitably in the compounds of Formula (III), X² is absent. Suitably in the compounds of Formula (III), X² is N. Suitably in the compounds of Formula (III), X² is S. Suitably in the compounds of Formula (III), X² is O.

Suitably in the compounds of Formula (III), Y² is CH. Suitably in the compounds of Formula (III), Y² is N.

Suitably in the compounds of Formula (III), R²³ is absent or selected from:
H,
CH₃,
—CH₂CH₃,
—CH(CH₃)₂, and
cyclopropyl.
   Suitably in the compounds of Formula (III), R²⁴ is selected from:
Cl,
Br,
I,
C₁₋₄alkyl,
C₁₋₄alkyl substituted from 1 to 3 times by F,
cyclopropyl;
methylcyclopropyl,
cyclo butyl,
azetidinyl,
methylazetidinyl, and
pyrrolidinyl.

Suitably in the compounds of Formula (III), A² is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S.

Suitably in the compounds of Formula (III), R²¹ and R²² are independently selected from:
H,
OS(O)₂NH₂,
—S(O)₂CH₃,
—OH,
—CN,
F,
tetrazolyl
methyltetrazolyl,
cyclopropyl,
morpholinyl,
tetrazolyl,
methyltetrazolyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cyclopropyl, cyclopentyl, —S(O)₂CH₃, —NH₂, —N(H)C₁₋₄alkyl, —N(H)C₁₋₄alkyl where alkyl is substituted with from 1 to 5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently selected from: —OH, oxo, fluoro, C₁₋₄alkoxy, cyclopropyl, —NH₂, —N(H)C₁₋₄alkyl, —N(H)C₁₋₄alkyl where the alkyl is substituted with from 1 to 5 fluoro, —N(C₁₋₄alkyl)₂, —N(C₁₋₄alkyl)₂ where the alkyls are independently substituted with from 1 to 7 fluoro, —S(O)₂OH₃, —S(O)₂NH₂, and —S(O)₂N(H)C₁₋₄alkyl,
N(H)C₁₋₆alkyl, and
N(H)C₁₋₆alkyl substituted with from one to six substituents independently selected from: —OH, oxo, fluoro, and —S(O)₂CH₃.

Suitably in the compounds of Formula (III), the moiety —X²R²³R²⁴ is selected from: bromo, cyclopropyl, methylcyclopropyl, cyclobutyl, azetidinyl, methylazetidinyl, —NHCH(CH₃)₂, —N(CH₃)CH(CH₃)₂, —NHCH₃, —N(CH₃)₂, —CF(CH₃)₂, —C(CH₃)₃, —CH(CH₃)₂, pyrrolidinyl, —N(CH₃)cyclopropyl, —N(cyclopropyl)₂, —NCH(CH₃)₂CH(CH₃)₂, —N(CH₃)C(CH₃)₃, —SCH₃, and —OCH₃.

Suitably in the compounds of Formula (III), A² is selected from: cyclohexyl, cyclobutyl, bicyclopentanyl, spiroheptanyl, pyrrolidinyl, tetrahydropyranyl, and piperidinyl.

Suitably in the compounds of Formula (III), R²¹ and R²² are independently selected from: hydrogen, fluoro, —OH, —CH₃, —OCH₂CH₂OH, oxo, —CH₂OH, —C(CH₃)₂OH, —NHCH(CH₃)CHF₂, —CH(cyclopropyl)OH, —CH(OH)CH₂S(O)₂CH₃, tetrazolyl, methyltetrazolyl, difluoroazetidinyl, fluoroazetidinyl, azetidinyl and —CH(OH)CF₃.

Included in the presently invented compounds of Formula (I) are compounds of Formula (IV):

[chemical structure]

wherein:
R³⁰ is selected from: bromo, cyclopropyl, methylcyclopropyl, cyclobutyl, azetidinyl, methylazetidinyl, —NHCH(CH₃)₂, —N(CH₃)CH(CH₃)₂, —NHCH₃, —N(CH₃)₂, —CF(CH₃)₂, —C(CH₃)₃, —CH(CH₃)₂, pyrrolidinyl, —N(CH₃)cyclopropyl, —N(cyclopropyl)₂, —NCH(CH₃)₂CH(CH₃)₂, —N(CH₃)C(CH₃)₃, —SCH₃, and —OCH₃;
Y³ is selected from: CH, and N;
A³ is selected from: cyclohexyl, cyclobutyl, bicyclopentanyl, spiroheptanyl, pyrrolidinyl, tetrahydropyranyl, and piperidinyl; and
R³¹ and R³² are independently selected from: hydrogen, fluoro, —OH, —CH₃, —OCH₂CH₂OH, oxo, —CH₂OH, —C(CH₃)₂OH, —NHCH(CH₃)CHF₂, —CH(cyclopropyl)OH, —CH(OH)CH₂S(O)₂CH₃, tetrazolyl, methyltetrazolyl, difluoroazetidinyl, fluoroazetidinyl, azetidinyl and —CH(OH)CF₃;
   and salts thereof.

This invention also relates to pharmaceutically acceptable salts of the compounds of Formula (IV).

Suitably in the compounds of Formula (IV), the moiety R³⁰ is selected from: bromo, cyclopropyl, methylcyclopropyl, cyclobutyl, azetidinyl, methylazetidinyl, —NHCH(CH₃)₂, —N(CH₃)CH(CH₃)₂, —NHCH₃, —N(CH₃)₂, —CF(CH₃)₂, —C(CH₃)₃, —CH(CH₃)₂, pyrrolidinyl, —N(CH₃)cyclopropyl, —N(cyclopropyl)₂, —NCH(CH₃)₂CH(CH₃)₂, —N(CH₃)C(CH₃)₃, —SCH₃, and —OCH₃.

Suitably in the compounds of Formula (IV), A³ is selected from: cyclohexyl, cyclobutyl, bicyclopentanyl, spiroheptanyl, pyrrolidinyl, tetrahydropyranyl, and piperidinyl.

Suitably in the compounds of Formula (IV), R³¹ and R³² are independently selected from: hydrogen, fluoro, —OH, —CH₃, —OCH₂CH₂OH, oxo, —CH₂OH, —C(CH₃)₂OH, —NHCH(CH₃)CHF₂, —CH(cyclopropyl)OH, —CH(OH)CH₂S(O)₂CH₃, tetrazolyl, methyltetrazolyl, difluoroazetidinyl, fluoroazetidinyl, azetidinyl and —CH(OH)CF₃.

Included in the presently invented compounds of Formula (I) are:
2-Bromo-N-(trans)-4-(2-hydroxypropan-2-yl)cyclohexyl)thieno[3,2-b]pyridine-6-carboxamide;
2-Cyclopropyl-N-(trans)-4-(2-hydroxypropan-2-yl)cyclohexyl)thieno[3,2-b]pyridine-6-carboxamide;
2-Bromo-N-(cis)-3-hydroxy-3-methylcyclobutyl)thieno[3,2-b]pyridine-6-carboxamide;
2-Cyclopropyl-N-(cis)-3-hydroxy-3-methylcyclobutyl)thieno[3,2-b]pyridine-6-carboxamide;
N-(trans)-4-(2-Hydroxypropan-2-yl)cyclohexyl)-2-(isopropylamino)thiazolo[4,5-b]pyridine-6-carboxamide;
N-(trans)-4-(2-Hydroxypropan-2-yl)cyclohexyl)-2-(isopropyl(methyl)amino)thiazolo[4,5-b]pyridine-6-carboxamide;
N-(trans)-4-(2-Hydroxypropan-2-yl)cyclohexyl)-2-(methylamino)thiazolo[4,5-b]pyridine-6-carboxamide;
2-Cyclopropyl-N-(trans)-4-(2-hydroxypropan-2-yl)cyclohexyl)thiazolo[4,5-b]pyridine-6-carboxamide;
2-(Dimethylamino)-N-(trans)-4-(2-hydroxypropan-2-yl)cyclohexyl)thiazolo[4,5-b]pyridine-6-carboxamide;
N-(trans-4-(2-Hydroxypropan-2-yl)cyclohexyl)-2-((1S,2R)-2-methylcyclopropyl)thieno[3,2-b]pyridine-6-carboxamide;
N-(trans-4-(2-hydroxypropan-2-yl)cyclohexyl)-2-((1R,2S)-2-methylcyclopropyl)thieno[3,2-b]pyridine-6-carboxamide;
2-Bromo-N-(3-(2-hydroxypropan-2-yl)bicyclo[1,1,1]pentan-1-yl)thieno[3,2-b]pyridine-6-carboxamide;
2-Cyclopropyl-N-(3-(2-hydroxypropan-2-yl)bicyclo[1.1.1]pentan-1-yl)thieno[3,2-b]pyridine-6-carboxamide;
2-Cyclopropyl-N-(3-(2-hydroxypropan-2-yl)bicyclo[1.1.1]pentan-1-yl)thiazolo[4,5-b]pyridine-6-carboxamide;
2-Cyclobutyl-N-((trans)-4-(2-hydroxypropan-2-yl)cyclohexyl)thiazolo[4,5-b]pyridine-6-carboxamide;
2-cyclopropyl-N-(6-(2-hydroxypropan-2-yl)spiro[3.3]heptan-2-yl)thiazolo[4,5-b]pyridine-6-carboxamide;
2-Cyclopropyl-N-((trans)-4-hydroxycyclohexyl)thiazolo[4,5-b]pyridine-6-carboxamide;
2-Cyclopropyl-N-((trans)-4-hydroxy-4-methylcyclohexyl)thiazolo[4,5-b]pyridine-6-carboxamide;
2-Cyclopropyl-N-((trans)-4-(2-hydroxyethoxy)cyclohexyl)thiazolo[4,5-b]pyridine-6-carboxamide;
(S)-2-Cyclopropyl-N-(2-oxopyrrolidin-3-yl)thiazolo[4,5-b]pyridine-6-carboxamide;
2-Cyclopropyl-N-((trans)-4-(hydroxymethyl)cyclohexyl)thiazolo[4,5-b]pyridine-6-carboxamide;
2-Cyclopropyl-N-((trans)-4-(3,3-difluoroazetidin-1-yl)cyclohexyl)thiazolo[4,5-b]pyridine-6-carboxamide;
2-Cyclopropyl-N-((trans)-3-(2-hydroxypropan-2-yl)cyclobutyl)thiazolo[4,5-b]pyridine-6-carboxamide;
2-Cyclopropyl-N-(trans-4-((1,1-difluoropropan-2-yl)amino)cyclohexyl)thiazolo[4,5-b]pyridine-6-carboxamide;
2-Cyclopropyl-N-((3R,6S)-6-(2-hydroxypropan-2-yl)tetrahydro-2H-pyran-3-yl)thiazolo[4,5-b]pyridine-6-carboxamide;
2-Cyclopropyl-N-((3S,6R)-6-(2-hydroxypropan-2-yl)tetrahydro-2H-pyran-3-yl)thiazolo[4,5-b]pyridine-6-carboxamide;
2-(2-Fluoropropan-2-yl)-N-(trans-4-(2-hydroxypropan-2-yl)cyclohexyl)thiazolo[4,5-b]pyridine-6-carboxamide;
2-Cyclopropyl-N-(trans-4-(cyclopropyl(hydroxy)methyl)cyclohexyl)thiazolo[4,5-b]pyridine-6-carboxamide;
2-(tert-Butyl)-N-(trans-4-(2-hydroxypropan-2-yl)cyclohexyl)thiazolo[4,5-b]pyridine-6-carboxamide;
2-cyclopropyl-N-(trans-4-(1-hydroxy-2-(methylsulfonyl)ethyl)cyclohexyl)thiazolo[4,5-b]pyridine-6-carboxamide;
2-(Azetidin-1-yl)-N-(trans-4-(2-hydroxypropan-2-yl)cyclohexyl)thiazolo[4,5-b]pyridine-6-carboxamide;
N-(trans-4-(2-Hydroxypropan-2-yl)cyclohexyl)-2-isopropylthiazolo[4,5-b]pyridine-6-carboxamide;
2-Cyclopropyl-N-(1-(1-methyl-1H-tetrazol-5-yl)piperidin-4-yl)thiazolo[4,5-b]pyridine-6-carboxamide;
2-Cyclopropyl-N-(trans-4-(2,2,2-trifluoro-1-hydroxyethyl)cyclohexyl)thiazolo[4,5-b]pyridine-6-carboxamide;
N-(trans-4-(2-Hydroxypropan-2-yl)cyclohexyl)-2-(pyrrolidin-1-yl)thiazolo[4,5-b]pyridine-6-carboxamide;
N-(trans-4-(2-Hydroxypropan-2-yl)cyclohexyl)-2-((S)-2-methylazetidin-1-yl)thiazolo[4,5-b]pyridine-6-carboxamide;
2-(Cyclopropyl(methyl)amino)-N-(trans-4-(2-hydroxypropan-2-yl)cyclohexyl)thiazolo[4,5-b]pyridine-6-carboxamide;
2-(Dicyclopropylamino)-N-(trans-4-(2-hydroxypropan-2-yl)cyclohexyl)thiazolo[4,5-b]pyridine-6-carboxamide;
2-(Diisopropylamino)-N-(trans-4-(2-hydroxypropan-2-yl)cyclohexyl)thiazolo[4,5-b]pyridine-6-carboxamide;
2-(tert-Butyl(methyl)amino)-N-(trans-4-(2-hydroxypropan-2-yl)cyclohexyl)thiazolo[4,5-b]pyridine-6-carboxamide;
N-((trans)-4-(2-Hydroxypropan-2-yl)cyclohexyl)-2-(methylthio)thiazolo[4,5-b]pyridine-6-carboxamide;
N-((trans)-4-(2-Hydroxypropan-2-yl)cyclohexyl)-2-methoxythiazolo[4,5-b]pyridine-6-carboxamide;
2-cyclopropyl-N-((3S,5S)-3,5-dihydroxycyclohexyl)thiazolo[4,5-b]pyridine-6-carboxamide;
(S)-2-Cyclopropyl-N-(6-(2-hydroxypropan-2-yl)spiro[3.3]heptan-2-yl)thiazolo[4,5-b]pyridine-6-carboxamide; and
(R)-2-cyclopropyl-N-(6-(2-hydroxypropan-2-yl)spiro[3.3]heptan-2-yl)thiazolo[4,5-b]pyridine-6-carboxamide;
and pharmaceutically acceptable salts thereof.

The skilled artisan will appreciate that salts, including pharmaceutically acceptable salts, of the compounds according to Formula (I) may be prepared. Indeed, in certain embodiments of the invention, salts including pharmaceutically-acceptable salts of the compounds according to Formula (I) may be preferred over the respective free or unsalted compound. Accordingly, the invention is further directed to salts, including pharmaceutically-acceptable salts, of the compounds according to Formula (I). The invention is further directed to free or unsalted compounds of Formula (I).

The salts, including pharmaceutically acceptable salts, of the compounds of the invention are readily prepared by those of skill in the art.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), b/s-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (I), or in any chemical structure illustrated herein, if not specified the structure is intended to encompass all individual stereoisomers and all mixtures thereof. Thus, compounds according to Formula (I) containing one or more chiral centers may be used as racemic mixtures, enantiomerically enriched mixtures, or as enantiomerically pure individual stereoisomers.

The compounds according to Formula (I) and pharmaceutically acceptable salts thereof may contain isotopically-labelled compounds, which are identical to those recited in Formula (I) and following, but for the fact that one or more atoms are replaced by an atom having an atomic mass or mass number different from the atomic mass or mass number usually found in nature. Examples of such isotopes include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Isotopically-labelled compounds, for example those into which radioactive isotopes such as ³H or ¹⁴C are incorporated, are useful in drug and/or substrate tissue distribution assays. Tritium, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), both are useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds can generally be prepared by substituting a readily available isotopically labelled reagent for a non-isotopically labelled reagent.

The compounds according to Formula (I) may also contain double bonds or other centers of geometric asymmetry. Where the stereochemistry of a center of geometric asymmetry present in Formula (I), or in any chemical structure illustrated herein, is not specified, the structure is intended to encompass the trans (E) geometric isomer, the cis (Z) geometric isomer, and all mixtures thereof. Likewise, all tautomeric forms are also included in Formula (I) whether such tautomers exist in equilibrium or predominately in one form.

The compounds of the invention may exist in solid or liquid form. In solid form, compound of the invention may exist in a continuum of solid states ranging from fully amorphous to fully crystalline. The term ‘amorphous’ refers to a state in which the material lacks long range order at the molecular level and, depending upon the temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may have the ability to crystallize in more than one form, a characteristic, which is known as polymorphism (“polymorphs”). Polymorphism generally can occur as a response to changes in temperature or pressure or both and can also result from variations in the crystallization process. Polymorphs can be distinguished by various physical characteristics known in the art such as x-ray diffraction patterns, solubility and melting point.

The compounds of Formula (I) may exist in solvated and unsolvated forms. As used herein, the term “solvate” refers to a complex of variable stoichiometry formed by a solute (in this invention, a compound of Formula (I) or a salt) and a solvent. Such solvents, for the purpose of the invention, may not interfere with the biological activity of the solute. The skilled artisan will appreciate that pharmaceutically acceptable solvates may be formed for crystalline compounds wherein solvent molecules are incorporated into the crystalline lattice during crystallization. The incorporated solvent molecules may be water molecules or non-aqueous such as ethanol, isopropanol, DMSO, acetic acid, ethanolamine, and ethyl acetate molecules. Crystalline lattice incorporated with water molecules are typically referred to as “hydrates”. Hydrates include stoichiometric hydrates as well as compositions containing variable amounts of water.

It is also noted that the compounds of Formula (I) may form tautomers. Tautomers' refer to compounds that are interchangeable forms of a particular compound structure, and that vary in the displacement of hydrogen atoms and electrons. Thus, two structures may be in equilibrium through the movement of TT electrons and an atom (usually H). For example, enols and ketones are tautomers because they are rapidly interconverted by treatment with either acid or base. It is understood that all tautomers and mixtures of tautomers of the compounds of the present invention are included within the scope of the compounds of the present invention.

While aspects for each variable have generally been listed above separately for each variable this invention includes those compounds in which several or each aspect in Formula (I) is selected from each of the aspects listed above. Therefore, this invention is intended to include all combinations of aspects for each variable.

### Definitions

It will be appreciated that the following definitions apply to each of the aforementioned formulae and to all instances of these terms, unless the context dictates otherwise.

“Alkyl” refers to a hydrocarbon chain having the specified number of “carbon atoms”. For example, C-j-Ce alkyl refers to an alkyl group having from 1 to 6 carbon atoms. Alkyl groups may be saturated, unsaturated, straight or branched. Representative branched alkyl groups have one, two, or three branches. Alkyl includes but is not limited to: methyl, ethyl, ethylene, ethynyl, propyl (n-propyl and isopropyl), butene, butyl (n-butyl, isobutyl, and t-butyl), pentyl and hexyl. Suitably the “alkyl” group is saturated. Suitably the “alkyl” group is unsaturated. Suitably the “alkyl” group is a straight chain. Suitably the “alkyl” group is branched.
“Alkoxy” refers to an —O-alkyl group wherein “alkyl” is as defined herein. For example, C₁-C₄alkoxy refers to an alkoxy group having from 1 to 4 carbon atoms. Representative branched alkoxy groups have one, two, or three branches. Examples of such groups include methoxy, ethoxy, propoxy, t-butoxy and butoxy.
“Cycloalkyl” and “Cycloalkane”, unless otherwise defined, refers to a saturated or unsaturated non-aromatic hydrocarbon ring system having from three to seven carbon atoms. Cycloalkyl groups are monocyclic or bicyclic ring systems (bicyclic ring systems include bridged ring systems and spiro ring systems). For example, C₃-C₇ cycloalkyl refers to a cycloalkyl group having from 3 to 7 member atoms. Examples of cycloalkyl as used herein include: cyclopropyl, cyclobutyl, cyclopentyl, cyclohexyl, cyclobutenyl, cyclopentenyl, cyclohexenyl, cycloheptyl, bicyclopentanyl, and spiro heptanyl. Suitably “cycloalkyl” includes: cyclopropyl, cyclobutyl, cyclohexyl, bicyclopentanyl, and spiro heptanyl. Suitably “cycloalkyl” is a saturated ring system. Suitably “cycloalkyl” is an unsaturated ring system. Suitably “cycloalkyl” is a monocyclic ring system. Suitably “cycloalkyl” is a bicyclic ring system. Suitably “cycloalkyl” is a bridged ring system. Suitably “cycloalkyl” is a spiro ring system.
“Halogen” refers to the halogen radicals fluoro, chloro, bromo, and iodo.
“Heteroaryl” and “heteroaromatic” refers to a monocyclic aromatic 4 to 8 member ring containing from 1 to 7 carbon atoms and containing from 1 to 4 heteroatoms, provided that when the number of carbon atoms is 3, the aromatic ring contains at least two heteroatoms. Heteroaryl groups containing more than one heteroatom may contain different heteroatoms. Heteroaryl includes: pyrrolyl, pyrazolyl, imidazolyl, oxazolyl, isoxazolyl, thiazolyl, isothiazolyl, furanyl, furazanyl, thienyl, triazolyl, pyridinyl, pyrimidinyl, pyridazinyl, pyrazinyl, triazinyl, and tetrazinyl.
“Heterocycle”, “Heterocycloalkyl” and “heterocyclic group” refers to a saturated or unsaturated non-aromatic monocyclic ring system containing 4 to 7 member atoms, of which 1 to 6 are carbon atoms and from 1 to 4 are heteroatoms. Heterocycloalkyl groups containing more than one heteroatom may contain different heteroatoms. “Heterocycle”, “heterocycloalkyl”, and “heterocyclic group” includes: pyrrolidinyl, tetrahydrofuranyl, dihydrofuranyl, pyranyl, tetrahydropyranyl, dihydropyranyl, tetrahydrothienyl, pyrazolidinyl, oxazolidinyl, oxetanyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, and azetidinyl. Suitably, “heterocycle”, “heterocycloalkyl”, and “heterocyclic group” includes: pyrrolidinyl, piperidinyl, and azetidinyl.
“Heteroatom” refers to a nitrogen, sulfur or oxygen atom.

### Abbreviations

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
Ac (acetyl);
Ac₂O (acetic anhydride);
ACN (acetonitrile);
AIBN (azobis(isobutyronitrile));
BINAP (2,2′-bis(diphenylphosphino)-1,1′-binaphthyl);
BMS (borane-dimethyl sulphide complex);
Bn (benzyl);
Boc (tert-Butoxycarbonyl);
Boc₂O (di-tert-butyl dicarbonate);
BOP (Benzotriazole-1-yl-oxy-tris-(dimethylamino)-phosphonium hexafluorophosphate);
CAN (cerric ammonium nitrate);
Cbz (benzyloxycarbonyl);
CSI (chlorosulfonyl isocyanate);
CsF (cesium fluoride);
DABCO (1,4-Diazabicyclo[2.2.2]octane);
DAST (Diethylamino)sulfur trifluoride);
DBU (1,8-Diazabicyclo[5.4.0]undec-7-ene);
DCC (Dicyclohexyl Carbodiimide);
DCE (1,2-dichloroethane);
DDQ (2,3-Dichloro-5,6-dicyano-1,4-benzoquinone);
ATP (adenosine triphosphate);
Bis-pinacolatodiboron (4,4,4′,4′,5,5,5′,5′-Octamethyl-2,2′-bi-1,3,2-dioxaborolane);
BSA (bovine serum albumin);
C18 (refers to 18-carbon alkyl groups on silicon in HPLC stationary phase);
CH₃CN (acetonitrile);
Cy (cyclohexyl);
DCM (dichloromethane);
DIEA (Hünig's base, N,N-Diisopropylethylamine, N-ethyl-N-(1-methylethyl)-2-pro panamine);
Dioxane (1,4-dioxane);
DMAP (4-dimethylaminopyridine);
DME (1,2-dimethoxyethane);
DMEDA (N,N-dimethylethylenediamine);
DMF (N,N-dimethylformamide);
DMSO (dimethylsulfoxide);
DPPA (diphenyl phosphoryl azide);
EDC (N-(3-dimethylaminopropyl)-N′ethylcarbodiimide);
EDTA (ethylenediaminetetraacetic acid);
EtOAc (ethyl acetate);
EtOH (ethanol);
Et₂O (diethyl ether);
HEPES (4-(2-hydroxyethyl)-1-piperazine ethane sulfonic acid);
HATU (0-(7-Azabenzotriazol-1-yl)-N,N,N′,N′-tetramethyluronium hexafluorophosphate, 1-((dimethylamino)(dimethyliminio)methyl)-1H-[1,2,3]triazolo[4,5-b]pyridine 3- oxide hexafluorophosphate(V));
HOAt (1-hydroxy-7-azabenzotriazole);
HOBt (1-hydroxybenzotriazole);
HOAc (acetic acid);
HPLC (high pressure liquid chromatography);
HMDS (hexamethyldisilazide);
IPA (isopropyl alcohol);
Indoline (2,3-dihydro-1H-indole);
KHMDS (potassium hexamethyldisilazide);
LAH (lithium aluminum hydride);
LDA (lithium diisopropylamide);
LHMDS (lithium hexamethyldisilazide)
MeOH (methanol);
MTBE (methyl tert-butyl ether);
mCPBA (m-chloroperoxybenzoic acid);
NaHMDS (sodium hexamethyldisilazide);
NBS (N-bromosuccinimide);
PE (petroleum ether);
Pd₂(dba)₃ (Tris(dibenzylideneacetone)dipalladium(0);
Pd(dppf)Cl₂⋅DCM Complex([1,1′-Bis(diphenylphosphino)ferrocene]dichloropalladium(II) ⋅dichloromethane complex);
PyBOP (benzotriazol-1-yl-oxytripyrrolidinophosphonium hexafluorophosphate);
PyBrOP (bromotripyrrolidinophosphonium hexafluorophosphate);
RP-HPLC (reverse phase high pressure liquid chromatography);
RT (room temperature);
Sat. (saturated)
SFC (supercritical fluid chromatography);
SGC (silica gel chromatography);
SM (starting material);
TLC (thin layer chromatography);
TEA (triethylamine);
TEMPO (2,2,6,6-Tetramethylpiperidine 1-oxyl, free radical);
TFA (trifluoroacetic acid); and
THF (tetrahydrofuran).

All references to ether are to diethyl ether and brine refers to a saturated aqueous solution of NaCl.

### COMPOUND PREPARATION

The compounds according to Formula (I) are prepared using conventional organic synthetic methods. A suitable synthetic route is depicted below in the following general reaction schemes. All of the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

As used in the Schemes below, the “r” groups, such as r¹ and r² represents all corresponding positional combinations on all of the Formulas disclosed herein. For example r¹ and r² represent R³⁰, and -AR³¹R³² of Formula (IV).

In one method of preparation, thieno[3,2-b]pyridine-6-carboxamides may be synthesized from 5-bromothiophene-2-carbaldehyde as shown in Scheme 1. First, Michael addition of DABCO to methyl acrylate, followed by aldol condensation of the in situ generated enolate with bromothiophene-2-carbaldehyde and subsequent elimination of DABCO affords the hydroxymethylacrylate. Then, acetylation of the alcohol provides the acetate. Subsequent, S_{N}2′ displacement of the acetate then gives the allylic amine. Oxidative cyclization of this amine thiophene, mediated via iodine, affords the bromothieno[3,2-b]pyridine-6-carboxester. Hydrolysis of this ester and amide coupling with various amines gives bromothieno[3,2-b]pyridine-6-carboxamides. Finally, Suzuki cross coupling of these bromides with various boronic acids affords the desired thieno[3,2-b]pyridine-6-carboxamides.

[chemical structure]

In another method of preparation, thiazolo[4,5-b]pyridine-6-carboxamides may be synthesized from methyl 6-amino-5-bromonicotinate as shown in Scheme 2. First, acylation of the aminopyridine with various acid chlorides affords amides as well as imide by-products. The mixture can be converted into the desired amides by hydrolysis of the imide by-products. Then, conversion of the carboxamides to the thiocarboxamides, employing Lawesson's reagent, and subsequent anion-mediated cyclization provides the thiazolo[4,5-b]pyridine-6-carboxyesters. Finally, hydrolysis of the esters and amide bond formation with various amines gives the desired thiazolo[4,5-b]pyridine-6-carboxamides.

[chemical structure]

### METHODS OF USE

The inventors have shown that inhibitors of Hematopoietic Prostaglandin D Synthase (H-PGDS) reduce muscle damage and preserve muscle function when administered prior to muscle injury in an in vivo assay for muscle function. Furthermore, the inventors have shown that when an H-PGDS inhibitor is administered after muscle damage in the same assay, recovery of muscle function is enhanced. These results support a role for the use of H-PGDS inhibitors in the treatment of muscle degenerative disorders and muscle injury.

In one aspect, the invention provides a method of treating a muscle degenerative disorder comprising administering to a human an H-PGDS inhibitor of Formula (I) or a pharmaceutically acceptable salt thereof.

In particular embodiments, the muscle degenerative disorder is muscular dystrophy, myotonic dystrophy, polymyositis, dermatomyositis, or inclusion body myositis.

For example, the compounds of Formula (I) or a pharmaceutically acceptable salt thereof may be used to treat a muscular dystrophy disorder selected from Duchenne MD, Becker MD, congenital MD (Fukuyama), Emery Dreifuss MD, limb girdle MD, and fascioscapulohumeral MD.

The compounds of Formula (I) or a pharmaceutically acceptable salt thereof may also be used to treat myotonic dystrophy type I (DM1 or Steinert's), myotonic dystrophy type II (DM2 or proximal myotonic myopathy), or congenital myotonia.

In some embodiments, the muscle injury is a surgery-related muscle injury, a traumatic muscle injury, a work-related skeletal muscle injury, or an overtraining-related muscle injury.

Non-limiting examples of surgery-related muscle injuries include muscle damage due to knee replacement, anterior cruciate ligament (ACL) repair, plastic surgery, hip replacement surgery, joint replacement surgery, tendon repair surgery, surgical repair of rotator cuff disease and injury, and amputation.

In one embodiment, the muscle injury is a surgery-related muscle injury and the treatment method provides for administration of at least one dose of an H-PGDS inhibitor of Formula (I) or a pharmaceutically acceptable salt thereof prior to the surgery (for example, within one day before the surgery) followed by periodic administration of a dose of the H-PGDS inhibitor during the recovery period.

In another embodiment, the muscle injury is a surgery-related muscle injury and the treatment method provides for administration of at least one high dose of an H-PGDS inhibitor of Formula (I) or a pharmaceutically acceptable salt thereof within one day to one week following the surgery.

In yet another embodiment, the muscle injury is a surgery-related muscle injury and the treatment method provides for administration of at least one high dose of an H-PGDS inhibitor of Formula (I) or a pharmaceutically acceptable salt thereof within one day to one week following the surgery, followed by periodic administration of a dose of the H-PGDS inhibitor during the recovery period.

Non-limiting examples of traumatic muscle injuries include battlefield muscle injuries, auto accident-related muscle injuries, and sports-related muscle injuries. Traumatic injury to the muscle can include lacerations, blunt force contusions, shrapnel wounds, muscle pulls or tears, burns, acute strains, chronic strains, weight or force stress injuries, repetitive stress injuries, avulsion muscle injury, and compartment syndrome.

In one embodiment, the muscle injury is a traumatic muscle injury and the treatment method provides for administration of at least one dose of an H-PGDS inhibitor of Formula (I) or a pharmaceutically acceptable salt thereof, immediately after the traumatic injury (for example, within one day of the injury) followed by periodic administration of a dose of the H-PGDS inhibitor during the recovery period.

Non-limiting examples of work-related muscle injuries include injuries caused by highly repetitive motions, forceful motions, awkward postures, prolonged and forceful mechanical coupling between the body and an object, and vibration.

Overtraining-related muscle injuries include unrepaired or under-repaired muscle damage coincident with a lack of recovery or lack of an increase of physical work capacity.

In an additional embodiment, the muscle injury is exercise or sports-induced muscle damage including exercise-induced delayed onset muscle soreness (DOMS).

In some embodiments, the invention encompasses a therapeutic combination in which the H-PGDS inhibitor of Formula (I) or a pharmaceutically acceptable salt thereof is administered in a subject in combination with the implantation of a biologic scaffold (e.g. a scaffold comprising extracellular matrix) that promotes muscle regeneration. Such scaffolds are known in the art. See, for example, Turner and Badylack (2012) Cell Tissue Res. 347(3):759-74 and U.S. Pat. No. 6,576,265. Scaffolds comprising non-crosslinked extracellular matrix material are preferred.

In another aspect, the invention provides a method of treating tendon damage where the method comprises administering a compound of Formula (I) or a pharmaceutically acceptable salt thereof to a subject in need thereof. In a particular embodiment, the invention includes a method of enhancing the formation of a stable tendon-bone interface. In a related embodiment, the invention provides a method of increasing the stress to failure of tendons, for example surgically-repaired tendons. In an additional embodiment, the invention provides a method of reducing fibrosis at the repair site for surgically-repaired tendons. In a particular embodiment, the invention provides a method of treating tendon damage associated with rotator cuff injury, or tendon damage associated with surgical repair of rotator cuff injury.

In another aspect, the invention provides a method of treating a disease state selected from: allergic diseases and other inflammatory conditions such as asthma, aspirin-exacerbated respiratory disease (AERD), cough, chronic obstructive pulmonary disease (including chronic bronchitis and emphysema), bronchoconstriction, allergic rhinitis (seasonal or perennial), vasomotor rhinitis, rhinoconjunctivitis, allergic conjunctivitis, food allergy, hypersensitivity lung diseases, eosinophilic syndromes including eosinophilic asthma, eosinophilic pneumonitis, eosinophilic oesophagitis, eosinophilic granuloma, delayed-type hypersensitivity disorders, atherosclerosis, rheumatoid arthritis, pancreatitis, gastritis, inflammatory bowel disease, osteoarthritis, psoriasis, sarcoidosis, pulmonary fibrosis, respiratory distress syndrome, bronchiolitis, sinusitis, cystic fibrosis, obesity, actinic keratosis, skin dysplasia, chronic urticaria, eczema and all types of dermatitis including atopic dermatitis or contact dermatitis in a subject in need thereof comprising administering to the subject a therapeutically effective amount of a compound of Formula (I) or a pharmaceutically acceptable salt thereof.

The methods of treatment of the invention comprise administering a safe and effective amount of a compound of Formula (I), or a pharmaceutically acceptable salt thereof to a mammal, suitably a human, in need thereof.

As used herein, “treat”, and derivatives thereof, in reference to a condition means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The term “treating” and derivatives thereof refers to therapeutic therapy. Therapeutic therapy is appropriate to alleviate symptoms or to treat at early signs of disease or its progression.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of Formula (I), or a pharmaceutically acceptable salt thereof, means an amount of the compound sufficient to treat the patient's condition but low enough to avoid serious side effects (at a reasonable benefit/risk ratio) within the scope of sound medical judgment. A safe and effective amount of the compound will vary with the particular route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient”, and derivatives thereof refers to a human or other mammal, suitably a human.

The subject to be treated in the methods of the invention is typically a mammal in need of such treatment, preferably a human in need of such treatment.

### COMPOSITIONS

The pharmaceutically active compounds within the scope of this invention are useful as inhibitors of H-PGDS in mammals, particularly humans, in need thereof.

The present invention therefore provides a method of treating neurodegenerative diseases, musculoskeletal diseases and other conditions requiring H-PGDS inhibition, which comprises administering an effective amount of a compound of Formula (I) or a pharmaceutically acceptable salt thereof. The compounds of Formula (I) also provide for a method of treating the above indicated disease states because of their demonstrated ability to act as H-PGDS inhibitors. The drug may be administered to a patient in need thereof by any conventional route of administration, including, but not limited to, intravenous, intramuscular, oral, topical, subcutaneous, intradermal, intraocular and parenteral. Suitably, a H-PGDS inhibitor may be delivered directly to the brain by intrathecal or intraventricular route, or implanted at an appropriate anatomical location within a device or pump that continuously releases the H-PGDS inhibitor drug.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical compositions are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-500 mg/kg of active compound, preferably 0.001-100 mg/kg. When treating a human patient in need of a H-PGDS inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages, is preferred. Parenteral administration, at high dosages, however, also can be used when safe and convenient for the patient.

Optimal dosages to be administered may be readily determined by those skilled in the art, and will vary with the particular H-PGDS inhibitor in use, the strength of the preparation, the mode of administration, and the advancement of the disease condition. Additional factors depending on the particular patient being treated will result in a need to adjust dosages, including patient age, weight, diet, and time of administration.

When administered to prevent organ damage in the transportation of organs for transplantation, a compound of Formula (I) is added to the solution housing the organ during transportation, suitably in a buffered solution.

The method of this invention of inducing H-PGDS inhibitory activity in mammals, including humans, comprises administering to a subject in need of such activity an effective H-PGDS inhibiting amount of a pharmaceutically active compound of the present invention.

The invention also provides for the use of a compound of Formula (I) or a pharmaceutically acceptable salt thereof in the manufacture of a medicament for use as a H-PGDS inhibitor.

The invention also provides for the use of a compound of Formula (I) or a pharmaceutically acceptable salt thereof in the manufacture of a medicament for use in therapy.

The invention also provides for the use of a compound of Formula (I) or a pharmaceutically acceptable salt thereof in the manufacture of a medicament for use in treating musculoskeletal diseases such as Duchenne muscular dystrophy, spinal cord contusion injury, neuroinflammatory diseases such as multiple sclerosis or neurodegenerative diseases such as Alzheimer's disease or amyotrophic lateral sclerosis (ALS).

The invention also provides for a pharmaceutical composition for use as a H-PGDS inhibitor which comprises a compound of Formula (I) or a pharmaceutically acceptable salt thereof and a pharmaceutically acceptable carrier.

The invention also provides for a pharmaceutical composition for use in the treatment of cancer which comprises a compound of Formula (I) or a pharmaceutically acceptable salt thereof and a pharmaceutically acceptable carrier.

In addition, the pharmaceutically active compounds of the present invention can be co-administered with further active ingredients, such as other compounds known to treat cancer, or compounds known to have utility when used in combination with a H-PGDS inhibitor.

By the term “co-administration” as used herein is meant either simultaneous administration or any manner of separate sequential administration of a H-PGDS inhibiting compound, as described herein, and a further active agent or agents, known to be useful in the treatment of conditions in which a H-PGDS inhibitor is indicated. The term further active agent or agents, as used herein, includes any compound ortherapeutic agent known to or that demonstrates advantageous properties when administered to a patient in need of H-PGDS inhibition. Preferably, if the administration is not simultaneous, the compounds are administered in a close time proximity to each other. Furthermore, it does not matter if the compounds are administered in the same dosage form, e.g. one compound may be administered by injection and another compound may be administered orally.

The invention also relates to the use of a compound of Formula (I) or a pharmaceutically acceptable salt thereof in the preparation of a medicament for the treatment of neurodegenerative diseases, musculoskeletal diseases and diseases associated with H-PGDS inhibition.

The invention also provides a pharmaceutical composition comprising from 0.5 to 1,000 mg of a compound of Formula (I) or pharmaceutically acceptable salt thereof and from 0.5 to 1,000 mg of a pharmaceutically acceptable excipient.

Without further elaboration, it is believed that one skilled in the art can, using the preceding description, utilize the present invention to its fullest extent. The following Examples are, therefore, to be construed as merely illustrative and not a limitation of the scope of the present invention in anyway.

### EXPERIMENTAL DETAILS

### Examples

The following Examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### INTERMEDIATES

### Intermediate 1

### 2-Bromothieno[3,2-b]pyridine-6-carboxylic acid

[chemical structure]

### A. Methyl 2-((5-bromothiophen-2-yl)(hydroxy)methyl)acrylate

[chemical structure]

To a mixture of 5-bromothiophene-2-carbaldehyde (7.2 g, 37.7 mmol) and methyl acrylate (9.73 g, 113 mmol) was added DABCO (4.23 g, 37.7 mmol) in portions. After stirring at rt for 15 h, the reaction mixture was diluted with CH₂Cl₂ and water. The phases were separated and the aqueous phase was extracted 2× with CH₂Cl₂. The organic phases were combined, washed with brine, dried over Na₂SO₄, filtered, and concentrated. The residue was purified by silica gel chromatography, eluting with 0-30% EtOAc:hexanes gradient to give methyl 2-((5-bromothiophen-2-yl)(hydroxy)methyl)acrylate (9.97 g, 34.2 mmol, 91% yield) as a light yellow liquid. ¹H NMR (400 MHz, CDCl₃) δ ppm 6.93 (d, J=4 Hz, 1H), 6.73 (dd, J=4, 1 Hz, 1H), 6.40 (s, 1H), 5.98 (s, 1H), 5.67 (s, 1H), 3.80 (s, 3H), 3.42 (br. s., 1H).

### B. Methyl 2-(acetoxy(5-bromothiophen-2-yl)methyl)acrylate

[chemical structure]

To a solution of methyl 2-((5-bromothiophen-2-yl)(hydroxy)methyl)acrylate (9.95 g, 35.9 mmol) in CH₂Cl₂ (30 mL) was added acetic anhydride (5.5 g, 53.9 mmol), followed by DMAP (0.877 g, 7.18 mmol), slowly, in portions (exothermic). After ˜35 min, the reaction mixture was diluted with CH₂Cl₂ (50 mL) and washed with 10% NaHCO₃ aq. solution (100 mL). The aqueous phase was extracted 2× with CH₂Cl₂. The organic phases were combined, washed with brine, dried over Na₂SO₄, filtered, and concentrated. The residue was purified by silica gel chromatography, eluting with 0-30% EtOAc:hexanes gradient to give methyl 2-(acetoxy(5-bromothiophen-2-yl)methyl)acrylate (9.19 g, 27.4 mmol, 76% yield) as a light yellow liquid. ¹H NMR (400 MHz, CDCl₃) δ ppm 6.93 (d, J=4 Hz, 1H), 6.85 (d, J=4 Hz, 1H), 6.83 (s, 1H), 6.46 (s, 1H), 6.04 (s, 1H), 3.77 (s, 3H), 2.14 (s, 3H).

### C. (E)-Methyl 2-(aminomethyl)-3-(5-bromothiophen-2-yl)acrylate acetate

[chemical structure]

Into an ice-cold flask containing 100 mL of MeOH was bubbled NH₃ for ˜20 min. Then, a solution of methyl 2-(acetoxy(5-bromothiophen-2-yl)methyl)acrylate (2.1 g, 6.58 mmol) in MeOH (10 mL) was added dropwise. Bubbling of NH₃ was continued for another ˜15 min. The mixture was stirred in the ice bath for ˜15 min and then the ice bath was removed and the reaction mixture was stirred at rt. After ˜45 min, the reaction mixture was concentrated to dryness to give the crude acetic acid salt of (E)-methyl 2-(aminomethyl)-3-(5-bromothiophen-2-yl)acrylate as a light yellow solid (1.99 g, 3.85 mmol, 65% purity by LCMS). The crude product could be used as is or chromatographed. About 1 g of the crude product was preabsorbed onto Celite® and purified by silica gel chromatography, eluting with 30-100% ((3:1) EtOAc:EtOH):hexanes gradient over 5 min, followed by 100% (3:1) EtOAc:EtOH for 10 min, to give the acetic acid salt of (E)-methyl 2-(aminomethyl)-3-(5-bromothiophen-2-yl)acrylate as a light yellow solid (450 mg, 1.28 mmol, 20% yield; 40% overall yield for the reaction since only half of the material was purified). ¹H NMR (400 MHz, CDCl₃) δ ppm 7.73 (s, 1H), 7.11 (d, J=A Hz, 1H), 7.10 (d, J=4 Hz, 1H), 5.11 (br. s., 3H), 3.88 (s, 2H), 3.86 (s, 3H), 2.09 (s, 3H). MS: small m/z 276/278 (M+H) for Br isotopes.

### D. Methyl 2-bromothieno[3,2-b]pyridine-6-carboxylate

[chemical structure]

To a suspension of (E)-methyl 2-(aminomethyl)-3-(5-bromothiophen-2-yl)acrylate, acetic acid salt (450 mg, 1.34 mmol) in MeCN (10 mL) was added 1 eq of K₂CO₃ (185 mg, 1.338 mmol), followed by iodine (1359 mg, 5.35 mmol) in portions. After ˜5 min, 4 eq of K₂CO₃ (740 mg, 5.35 mmol) were added. After ˜30 min, additional iodine (1019 mg, 4.02 mmol) and K₂CO₃ (555 mg, 4.02 mmol) were added. After 90 min, an aqueous saturated solution of sodium thiosulfate was added (25 mL). The mixture was diluted with EtOAc (˜50 mL) and the phases were separated. The organic phase was washed 1× with aq. satd. thiosulfate and 1× with brine, dried over Na₂SO₄, filtered, and concentrated. The residue was purified by silica gel chromatography, eluting with 0-20% EtOAc:hexanes gradient to give methyl 2-bromothieno[3,2-b]pyridine-6-carboxylate (91 mg, 0.32 mmol, 24% yield). ¹H NMR (400 MHz, CDCl₃) δ ppm 9.26 (d, J=2 Hz, 1H), 8.72 (d, J=2 Hz, 1H), 7.67 (s, 1H), 4.02 (s, 3H). MS: m/z 272/274 (M+H) for Br isotopes.

### E. 2-Bromothieno[3,2-b]pyridine-6-carboxylic acid

[chemical structure]

To a suspension of methyl 2-bromothieno[3,2-b]pyridine-6-carboxylate (164 mg, 0.603 mmol) in 1:1 THF:MeOH (8 mL) was added a solution of NaOH (320 mg, 8.00 mmol) in water (4 mL). After ˜90 min, the reaction mixture was partially concentrated down to the aqueous phase. The leftover solution was acidified to pH˜3 with aq. HCl, which caused precipitation of a light brown solid. The mixture was extracted with EtOAc containing 10% MeOH. Some of the solids dissolved. The aqueous phase containing the undissolved solids was extracted 4× with EtOAc containing ˜10% MeOH. The combined organic phases were washed 1× with brine, dried over Na₂SO₄, filtered, and concentrated to give 2-bromothieno[3,2-b]pyridine-6-carboxylic acid (149 mg, 0.548 mmol, 91% yield) as a light brown solid. ¹H NMR (400 MHz, CD₃SOCD₃) δ ppm 13.49 (br. s., 1H), 9.09 (d, J=2 Hz, 1H), 9.02 (d, J=2 Hz, 1H), 7.91 (s, 1H). MS: m/z 258/260 (M+H) for Br isotopes.

### Intermediate 2

### 2-(Isopropylamino)thiazolo[4,5-b]pyridine-6-carboxylic acid

[chemical structure]

### A. Ethyl 2-(isopropylamino)thiazolo[4,5-b]pyridine-6-carboxylate

[chemical structure]

To an ice-cold solution of ethyl 6-amino-5-bromonicotinate (150 mg, 0.61 mmol) and 2-isothiocyanatopropane (62 mg, 0.61 mmol) in DMF (2 mL) was added NaH (60% in oil, 25 mg, 0.61 mmol). The reaction mixture was stirred in the cold bath for about 30 min and then it was heated at 75° C. over the weekend (63 h). Upon cooling, the reaction mixture was diluted with EtOAc and washed 2× with water and 1× with brine. The combined aqueous phases were back-extracted 1× with EtOAc. This EtOAc phase was washed 1× with brine. The organic phases were dried over Na₂SO₄, filtered, and concentrated. The residue was purified by silica gel chromatography, eluting with 5-70% EtOAc:hexanes gradient to give ethyl 2-(isopropylamino)thiazolo[4,5-b]pyridine-6-carboxylate (106 mg, 0.38 mmol, 62% yield) as a white solid. ¹H NMR (400 MHz, CD3SOCD₃) δ ppm 8.82 (br. s., 1H), 8.78 (d, J=2 Hz, 1H), 8.58 (d, J=2 Hz, 1H), 4.32 (q, J=7 Hz, 2H), 3.98-4.23 (m, 1H), 1.33 (t, J=7 Hz, 3H), 1.25 (d, J=6 Hz, 6H). MS: m/z 266 (M+H).

### B. 2-(Isopropylamino)thiazolo[4,5-b]pyridine-6-carboxylic acid

[chemical structure]

To a solution of ethyl 2-(isopropylamino)thiazolo[4,5-b]pyridine-6-carboxylate (100 mg, 0.377 mmol) in 1:1 MeOH:THF (8 mL) was added a solution of NaOH (183 mg, 4.58 mmol) in water (3 mL). The homogeneous mixture was stirred at rt. After ˜15 h, the reaction mixture was concentrated down to the aqueous phase, and the leftover solution was acidified with 6N HCl (0.8 mL). The mixture was initially extracted 3× with EtOAc. Then the aqueous phase was saturated with NaCl and re-extracted 5× with EtOAc containing ˜10% MeOH. All of the organic phases were combined, washed with a minimal amount of brine, dried over Na₂SO₄, filtered, and concentrated to give 2-(isopropylamino)thiazolo[4,5-b]pyridine-6-carboxylic acid (89 mg, 0.356 mmol, 95% yield) as a white solid. ¹H NMR (400 MHz, CD₃SOCD₃) δ ppm 9.77 (br. s., 1H), 8.79 (br. s, 1H), 8.71 (d, J=2 Hz, 1H), 4.15 (br. s., 1H), 1.28 (d, J=6 Hz, 6H). MS: m/z 238 (M+H).

### Intermediate 3

### 2-(Methylamino)thiazolo[4,5-b]pyridine-6-carboxylic acid

[chemical structure]

### A. Ethyl 2-(methylamino)thiazolo[4,5-b]pyridine-6-carboxylate

[chemical structure]

To an ice-cold solution of ethyl 6-amino-5-bromonicotinate (100 mg, 0.408 mmol) and isothiocyanatomethane (30 mg, 0.028 mL, 0.408 mmol) in DMF (2 mL) was added NaH (60% in oil) (16 mg, 0.408 mmol). The reaction mixture was stirred in the cold bath for ˜30 min and then it was heated at 75° C. for ˜65 h. The reaction mixture was diluted with EtOAc and washed 1× with water and 1× with brine. The combined aqueous phases were back-extracted 1× with EtOAc. This EtOAc phase was washed 1× with brine. The organic phases were combined, dried over Na₂SO₄, filtered, and concentrated. The residue was purified by silica gel chromatography, eluting with 5-60% ((3:1) EtOAc:EtOH):hexanes gradient to give ethyl 2-(methylamino)thiazolo[4,5-b]pyridine-6-carboxylate (47 mg, 0.182 mmol, 45% yield) as a light yellow solid. ¹H NMR (400 MHz, CD₃SOCD₃+D₂O) δ ppm 8.77 (d, J=2 Hz, 2H), 8.57 (br. s, 1H), 4.30 (q, J=7 Hz, 2H), 2.99 (s, 3H), 1.31 (t, J=7 Hz, 3H). MS: m/z 238 (M+H).

### B. 2-(Methylamino)thiazolo[4,5-b]pyridine-6-carboxylic acid

[chemical structure]

To a suspension of ethyl 2-(methylamino)thiazolo[4,5-b]pyridine-6-carboxylate (45 mg, 0.190 mmol) in THF (7 mL) was added potassium trimethylsilanolate (37 mg, 0.260 mmol). LCMS after 15 h showed no reaction. Then, MeOH (4 mL) and water (2 mL) were added, followed by a solution of NaOH (130 mg, 3.25 mmol) in water (3 mL). After ˜3 h, the reaction mixture was concentrated down to the aqueous phase. The leftover solution was slowly acidified with 6N HCl, and a solid precipitated out. The mixture was stirred for a few minutes, and then the precipitate was collected by filtration, washed sequentially with a minimal amount of water and then with hexanes, and dried under high vacuum to give 2-(methylamino)thiazolo[4,5-b]pyridine-6-carboxylic acid (26 mg, 0.118 mmol, 62% yield) as a light yellow solid. ¹H NMR (400 MHz, CD₃SOCD₃) d ppm 13.13 (br. s., 1H), 9.10 (br. s., 1H), 8.77 (d, J=2 Hz, 1H), 8.67 (br. s., 1H), 3.04 (d, J=4 Hz, 3H). MS: m/z 210 (M+H).

### Intermediate 4

### 2-Cyclopropylthiazolo[4,5-b]pyridine-6-carboxylic acid

[chemical structure]

### A. Methyl 5-bromo-6-(cyclopropanecarboxamido)nicotinate

[chemical structure]

To a slurry of methyl 6-amino-5-bromonicotinate (20 g, 87 mmol) in dichloromethane (120 mL) and pyridine (70.0 mL, 866 mmol) at 0° C. was added cyclopropanecarbonyl chloride (27.1 g, 23.60 mL, 260 mmol), via dropping addition funnel (the reaction mixture became a homogeneous solution during addition). The mixture was quenched by addition of MeOH (ca. 20 mL) and concentrated in vacuo (2× MeCN chase to remove pyridine). The residue was partitioned between EtOAc and water and the layers were separated. The organic layer was washed with water and brine, dried over Na₂SO₄, and concentrated in vacuo affording 34.3 g of a red syrup. Composition by LCMS is 97% bis amide, 3% mono amide. The syrup was taken up in THF (40 mL) and MeOH (40 mL) and the solution was cooled in an ice bath. A solution of sodium methoxide in MeOH (24.74 mL, 108 mmol) was added over ca. 5 min via dropping addition funnel. An orange-yellow ppt formed within 15 min. The mixture was quenched by addition of AcOH (6.44 mL, 113 mmol), causing the mixture to solidify. The solid mass was broken up with a spatula and stirred with water ca. 10 min. Solids were collected by filtration, washed with water, and dried overnight on the Buchner funnel to yield methyl 5-bromo-6-(cyclopropanecarboxamido)nicotinate (23.6 g, 79 mmol, 91% yield) as an off-white solid. ¹H NMR (400 MHz, CDCl₃) δ ppm 8.93 (d, J=2 Hz, 1H), 8.46 (d, J=2 Hz, 1H), 8.19 (br. s., 1H), 3.94 (s, 3H), 2.36-2.48 (m, 1H), 1.18-1.26 (m, 2H), 0.94-1.04 (m, 2H). MS: m/z 299/301 (M+H) for Br isotopes.

### B. Methyl 5-bromo-6-(cyclopropanecarbothioamido)nicotinate

[chemical structure]

To a suspension of methyl 5-bromo-6-(cyclopropanecarboxamido)nicotinate (16.17 g, 54.1 mmol) in THF (200 mL) was added Lawesson's reagent (24.05 g, 59.5 mmol) in portions. The heterogeneous mixture was heated at 65° C. under a reflux condenser for a total of 10 h, and then it stood at rt for ˜5 h. The reaction mixture was concentrated to dryness in a rotary evaporator. The residue was purified by silica gel chromatography, eluting with 0-30% EtOAc:hexanes gradient to give methyl 5-bromo-6-(cyclopropanecarbothioamido)nicotinate (15.25 g, 38.7 mmol, 72% yield based on 80% purity by ¹H NMR). ¹H NMR (400 MHz, CD₃SOCD₃) δ ppm 12.14 (s, 1H), 8.99 (d, J=2 Hz, 1H), 8.56 (d, J=2 Hz, 1H), 3.90 (s, 3H), 2.28-2.40 (m, 1H), 1.08-1.16 (m, 2H), 0.97-1.07 (m, 2H). MS: m/z 315/317 (M+H) for Br isotopes.

### C. Methyl 2-cyclopropylthiazolo[4,5-b]pyridine-6-carboxylate

[chemical structure]

To a solution of methyl 5-bromo-6-(cyclopropanecarbothioamido)nicotinate (1.48 g, 4.23 mmol) in DMSO (10 mL) was added NaH (60% in oil, 0.186 g, 4.65 mmol) in small portions. After addition, the reaction mixture was stirred at rt for 5 min, and then heated in a sealed tube at 70° C. for a total of 5.5 h. Upon cooling, the reaction mixture was diluted with water and extracted 1× with EtOAc. The aqueous phase was back-extracted 2× with EtOAc. The combined EtOAc phases were washed with a small amount of brine, dried over Na₂SO₄, filtered, and concentrated. The residue was purified by silica gel chromatography, eluting with 0-40% EtOAc:hexanes gradient to give methyl 2-cyclopropylthiazolo[4,5-b]pyridine-6-carboxylate (624 mg, 2.53 mmol, 60% yield). ¹H NMR (400 MHz, CD₃SOCD₃) δ ppm 9.08-9.10 (m, 2H), 3.92 (s, 3H), 2.62-2.73 (m, 1H), 1.32-1.41 (m, 2H), 1.24-1.31 (m, 2H). MS: m/z 235 (M+H).

### D. 2-Cyclopropylthiazolo[4,5-b]pyridine-6-carboxylic acid

[chemical structure]

To a solution of methyl 2-cyclopropylthiazolo[4,5-b]pyridine-6-carboxylate (620 mg, 2.65 mmol) in 1:1 THF:MeOH (20 mL) was added a solution of NaOH (1059 mg, 26.5 mmol) in water (10 mL). After ˜1 h, the reaction mixture was concentrated under vacuo, down to the aqueous phase. The remaining aqueous solution was diluted with water (˜10 mL) and stirred. It was then acidified slowly to pH˜3 with 6N HCl. Heavy precipitation occurred. After a few minutes, the solids were collected by filtration, washed sequentially with water and with hexanes, and dried under high vacuum to give 2-cyclopropylthiazolo[4,5-b]pyridine-6-carboxylic acid (524 mg, 2.26 mmol, 85% yield) as a light yellow solid. ¹H NMR (400 MHz, CD3SOCD₃) δ ppm 13.46 (br s, 1H), 9.07 (d, J=2 Hz, 1H), 9.04 (d, J=2 Hz, 1H), 2.67 (tt, J=8, 5 Hz, 1H), 1.32-1.40 (m, 2H), 1.23-1.31 (m, 2H). MS: m/z 221 (M+H).

### Intermediate 5

### 2-(3-Aminobicyclo[1.1.1]pentan-1-yl)propan-2-ol hydrochloride

[chemical structure]

### A. tert-Butyl (3-(2-hydroxypropan-2-yl)bicyclo[1.1.1]pentan-1-yl)carbamate

[chemical structure]

To a stirring solution of methyl 3-((tert-butoxycarbonyl)amino)bicyclo[1,1,1]pentane-1-carboxylate (0.2 g, 0.829 mmol) in THF (3.0 mL), under nitrogen, in an ice bath was slowly added methylmagnesium bromide (3 M in Et₂O, 5 mL, 3.32 mmol). After addition, the reaction mixture was stirred in the ice bath for ˜10 min and then at rt for ˜30 mins. The reaction was quenched with sat. aq. ammonium chloride and extracted with ethyl acetate. The organic phase was washed with brine, dried over sodium sulfate, filtered, and concentrated under reduced pressure to give crude tert-butyl (3-(2-hydroxypropan-2-yl)bicyclo[1,1,1]pentan-1-yl)carbamate (187 mg, 0.775 mmol, 93% yield) as a colorless oil. ¹H NMR (400 MHz, CD3SOCD₃) δ ppm 7.39 (br. s., 1H), 4.11 (s, 1H), 1.70 (s, 6H), 1.37 (s, 9H), 1.03 (s, 6H).

### B. 2-(3-Aminobicyclo[1,1,1]pentan-1-yl)propan-2-ol hydrochloride

[chemical structure]

To a solution of tert-butyl (3-(2-hydroxypropan-2-yl)bicyclo[1,1,1]pentan-1-yl)carbamate (165 mg, 0.684 mmol) in dioxane (3 mL) was added 4M HCl in dioxane (6 mL, 24.00 mmol). The mixture was stirred at rt for ˜4 h, and then it was concentrated under vacuum. The residue was treated with CH₂Cl₂ and concentrated (3×). The residue was dissolved in MeOH and concentrated (2×), then dried under high vacuum to give crude 2-(3-aminobicyclo[1.1.1]pentan-1-yl)propan-2-ol hydrochloride (164 mg, 0.646 mmol, ˜95% yield based on 70-80% purity by ¹H NMR) as a light beige foamy solid. ¹H NMR (400 MHz, CD3SOCD₃) δ ppm 8.70 (brs, 3H), 4.36 (s, 1H), 1.80 (s, 6H), 1.05 (s, 6H).

### Intermediate 6

### 2-Cyclobutylthiazolo[4,5-b]pyridine-6-carboxylic acid

[chemical structure]

### A. 5-Bromo-6-(cyclobutanecarboxamido)nicotinic acid

[chemical structure]

To a suspension of methyl 6-amino-5-bromonicotinate (400 mg, 1.73 mmol) in CH₂Cl₂ (3 mL), cooled in an ice bath, was added TEA (0.241 mL, 1.731 mmol), followed by dropwise addition of a solution of cyclobutanecarbonyl chloride (0.198 mL, 1.731 mmol) in CH₂Cl₂ (2 mL). The mixture was allowed to warm up to rt. LCMS after ˜15 h shows 43% starting material, 18% mono-amide and 33% bis-amide. Additional TEA (0.241 mL, 1.731 mmol) was then added, followed by additional cyclobutanecarbonyl chloride (0.198 mL, 1.731 mmol). LCMS after ˜1 h shows 14% mono-amide product and 84% bis-amide. The reaction mixture was then concentrated to dryness. To hydrolyze the bis-amide to the mono-amide, the crude residue was dissolved 1:1 MeOH:THF (8 mL) and treated with a solution of NaOH (208 mg, 5.19 mmol) in water (4 mL). After ˜45 min, the reaction mixture was acidified to pH˜3 with 6N HCl. The reaction mixture was concentrated in vacuo, down to the aqueous phase. A solid precipitated out, and it was collected by filtration, washed sequentially with water and with hexanes, and dried under high vacuum at 60° C. over the weekend to give crude 5-bromo-6-(cyclobutanecarboxamido)nicotinic acid (236 mg, 0.71 mmol, 41% yield) as a yellow solid. ¹H NMR (400 MHz, CD3SOCD₃) δ ppm 13.55-13.74 (m, 1H), 10.23 (s, 1H), 8.87 (d, J=2 Hz, 1H), 8.44 (d, J=2 Hz, 1H), 3.34 (m, 1H, partially overlapping water peak), 2.19-2.32 (m, 2H), 2.06-2.18 (m, 2H), 1.89-2.03 (m, 1H), 1.75-1.88 (m, 1H). MS: m/z 299/301 (M+H).

### B. Methyl 5-bromo-6-(cyclobutanecarboxamido)nicotinate

[chemical structure]

To a suspension of the crude 5-bromo-6-(cyclobutanecarboxamido)nicotinic acid from the previous step (236 mg, 0.71 mmol) in MeOH (10 mL) was added trimethylsilyldiazomethane (2M in Et₂O) (0.400 mL, 0.8 mmol). The reaction was monitored by LCMS and additional trimethylsilyldiazomethane was added until full conversion to the methyl ester was observed. The reaction mixture was then concentrated to dryness in a rotary evaporator to give crude methyl 5-bromo-6-(cyclobutanecarboxamido)nicotinate (235 mg, 0.675 mmol, 39% yield for the last 3 steps) as a yellow solid. ¹H NMR (400 MHz, CD3SOCD₃) δ ppm 10.27 (s, 1H), 8.90 (d, J=2 Hz, 1H), 8.48 (d, J=2 Hz, 1H), 3.89 (s, 3H), 3.35-3.42 (m, 1H), 2.18-2.32 (m, 2H), 2.06-2.18 (m, 2H), 1.90-2.03 (m, 1H), 1.74-1.88 (m, 1H). MS: m/z 313/314 (M+H) for Br isotopes.

### C. Methyl 5-bromo-6-(cyclobutanecarbothioamido)nicotinate

[chemical structure]

To a slurry of the crude methyl 5-bromo-6-(cyclobutanecarboxamido)nicotinate from the previous step (235 mg, 0.75 mmol) in THF (5 mL) was added Lawesson's reagent (334 mg, 0.825 mmol). The heterogeneous mixture was heated in a sealed tube at 65° C. for 8 h. Upon cooling, the reaction mixture was concentrated to dryness in a rotary evaporator. The residue was purified by silica gel chromatography, eluting with 0-30% EtOAc:hexanes gradient to give methyl 5-bromo-6-(cyclobutanecarbothioamido)nicotinate (195 mg, 0.551 mmol, 73% yield based on 93% purity by ¹H NMR) as a yellow solid. ¹H NMR (400 MHz, CD3SOCD₃) δ ppm 11.76 (s, 1H), 8.99 (d, J=2 Hz, 1H), 8.56 (d, J=2 Hz, 1H), 3.91 (s, 3H), 3.64 (m, 1H), 2.32-2.47 (m, 2H), 2.21 (m, 2H), 1.86-1.98 (m, 1H), 1.71-1.84 (m, 1H). MS: m/z 329/331 (M+H) for Br isotopes.

### D. Methyl 2-cyclobutylthiazolo[4,5-b]pyridine-6-carboxylate

[chemical structure]

To a solution of methyl 5-bromo-6-(cyclobutanecarbothioamido)nicotinate (192 mg, 0.583 mmol) in DMSO (3 mL) was added NaH (60% in oil, 26 mg, 0.642 mmol). The mixture was stirred at rt for ˜5 min, and then it was heated at 70° C. in a sealed tube for 8 h. Upon cooling, the reaction mixture was diluted with water and extracted with EtOAc. The EtOAc phase was washed with brine, dried over Na₂SO₄, filtered, and concentrated. The residue was purified by silica gel chromatography, eluting with 0-30% EtOAc:hexanes gradient to give methyl 2-cyclobutylthiazolo[4,5-b]pyridine-6-carboxylate (91 mg, 0.348 mmol, 60% yield). ¹H NMR (400 MHz, CD3SOCD₃) δ ppm 9.16-9.18 (m, 1H), 9.14-9.16 (m, 1H), 4.12 (m, 1H), 3.93 (s, 3H), 2.52-2.50 (m, 2H; overlapping with DMSO solvent peak), 2.37-2.48 (m, 2H), 2.05-2.19 (m, 1H), 1.93-2.03 (m, 1H). MS: m/z 249 (M+H).

### E. 2-Cyclobutylthiazolo[4,5-b]pyridine-6-carboxylic acid

[chemical structure]

To a solution of methyl 2-cyclobutylthiazolo[4,5-b]pyridine-6-carboxylate (88 mg, 0.354 mmol) in 1:1 THF:MeOH (8 mL) was added a solution of NaOH (230 mg, 5.75 mmol) in water (4 mL). After ˜45 min, the reaction mixture was concentrated under vacuo, down to the aqueous phase. The remaining aqueous solution was diluted with water (˜10 mL) and stirred. It was then acidified slowly to pH˜3 with 6N HCl. Heavy precipitation occurred. After a few minutes, the solids were collected by filtration, washed sequentially with water and then with hexanes, and dried under high vacuum to give 2-cyclobutylthiazolo[4,5-b]pyridine-6-carboxylic acid (63 mg, 0.255 mmol, 72% yield) as a light yellow solid. ¹H NMR (400 MHz, CD3SOCD₃) δ ppm 13.51 (br s, 1H), 9.13 (d, J=2 Hz, 1H), 9.11 (d, J=2 Hz, 1H), 4.11 (m, 1H), 2.37-2.49 (m, 4H), 2.05-2.19 (m, 1H), 1.91-2.04 (m, 1H). MS: m/z 235 (M+H).

### Intermediate 7

### Methyl 2-(2-fluoropropan-2-yl)thiazolo[4,5-b]pyridine-6-carboxylate

[chemical structure]

### A. Methyl 5-bromo-6-(2-fluoro-2-methylpropanamido)nicotinate

[chemical structure]

To a stirred solution of 2-fluoro-2-methylpropanoic acid (0.964 g, 9.09 mmol) in dichloromethane (10 mL), at rt, was added oxalyl chloride (1.59 mL, 18.18 mmol), followed by 4 drops of DMF. The reaction mixture was heated at reflux overnight, and afterwards the bulk of dichloromethane was distilled off at 45° C. without using vacuum. The remaining liquid was added dropwise, at rt, to a stirred solution of methyl 6-amino-5-bromonicotinate (1.4 g, 6.06 mmol) in pyridine (10 mL), and the mixture was stirred at rt overnight. The reaction mixture was then concentrated under vacuum. The residue was purified by silica gel chromatography, eluting with 0-70% EtOAc:hexanes gradient to give methyl 5-bromo-6-(2-fluoro-2-methylpropanamido)nicotinate (1.3 g, 3.87 mmol, 64% yield). ¹H NMR (400 MHz, CD3SOCD₃) δ: 10.53 (s, 1H), 8.96 (d, J=2 Hz, 1H), 8.54 (d, J=2 Hz, 1H), 3.91 (s, 3H), 1.60 (d, J=22 Hz, 6H). MS: m/z 319/321 (M+H) for Br isotopes.

### B. Methyl 5-bromo-6-(2-fluoro-2-methylpropanethioamido)nicotinate

[chemical structure]

A mixture of methyl 5-bromo-6-(2-fluoro-2-methylpropanamido)nicotinate (0.70 g, 2.193 mmol) and Lawesson's reagent (1.065 g, 2.63 mmol) in THF (14 mL) was heated in a sealed tube at 65° C. for 10 h. Upon cooling, the reaction mixture was concentrated under vacuum. The residue was purified by silica gel chromatography, eluting with 0-70% EtOAc:hexanes gradient to give methyl 5-bromo-6-(2-fluoro-2-methylpropanethioamido)nicotinate (0.560 g, 1.587 mmol, 72% yield). ¹H NMR (400 MHz, CD3SOCD₃) δ: 12.01 (d, J=5 Hz, 1H), 9.03 (d, J=2 Hz, 1H), 8.60 (d, J=2 Hz, 1H), 3.92 (s, 3H), 1.78 (d, J=22 Hz, 6H). MS: m/z 335/337 (M+H) for Br isotopes.

### C. Methyl 2-(2-fluoropropan-2-yl)thiazolo[4,5-b]pyridine-6-carboxylate

[chemical structure]

To a stirred solution of methyl 5-bromo-6-(2-fluoro-2-methylpropanethioamido)nicotinate (0.550 g, 1.641 mmol) in DMSO (5.5 mL), at rt, was added sodium hydride (0.072 g, 1.805 mmol). The mixture was then heated for 8 hours at 70° C. Upon cooling, the reaction mixture was poured into 1N HCl (100 mL) and extracted with EtOAc (2×100 mL). The combined EtOAc layers were washed with water (100 mL) and brine (100 mL), dried over Na₂SO₄, and concentrated under vacuum. The residue was purified by silica gel chromatography, eluting with 0-70% EtOAc:hexanes gradient to give methyl 2-(2-fluoropropan-2-yl)thiazolo[4,5-b]pyridine-6-carboxylate (0.30 g, 1.121 mmol, 68% yield). ¹H NMR (400 MHz, CD3SOCD₃) δ: 9.28 (d, J=2 Hz, 1H), 9.20 (d, J=2 Hz, 1H), 3.95 (s, 3H), 1.87 (d, J=22 Hz, 6H). MS: m/z 255 (M+H).

### Intermediate 8

### 2-Cyclopropyl-N-((trans)-4-formylcyclohexyl)thiazolo[4,5-b]pyridine-6-carboxamide

[chemical structure]

To an ice-cold suspension of 2-cyclopropyl-N-((trans)-4-(hydroxymethyl)cyclohexyl)thiazolo[4,5-b]pyridine-6-carboxamide (65 mg, 0.180 mmol, Example 20) in CH₂Cl₂ (4 mL) and DMSO (1 mL) was added DIEA (0.126 mL, 0.722 mmol), followed by a solution of pyridine⋅sulfurtrioxide (45% technical grade, 115 mg, 0.722 mmol) in DMSO (1 mL). After ˜5 min, the ice bath was removed, and the mixture was stirred at rt. After ˜30 min, the reaction mixture was diluted with EtOAc and washed 2× with water and 1× with brine, and then dried over Na₂SO₄, filtered, and concentrated.

The reaction was repeated using the same conditions with another 32 mg (0.089 mmol) of 2-cyclopropyl-N-((trans)-4-(hydroxymethyl)cyclohexyl)thiazolo[4,5-b]pyridine-6-carboxamide.

The crude products, after work-up, from both reactions were combined and purified by silica gel chromatography, eluting with 5-50% (3:1 EtOAc:EtOH):hexanes gradient to give 2-cyclopropyl-N-((trans)-4-formylcyclohexyl)thiazolo[4,5-b]pyridine-6-carboxamide (72 mg, 0.144 mmol, ˜54% yield based on 66% purity by ¹H NMR) as a white solid. The ¹H NMR (CD3SOCD₃) shows a major and a minor component (˜2:1). The ¹H NMR signals for the major component are consistent with the aldehyde product. ¹H NMR (400 MHz, CD3SOCD₃) δ ppm 9.60 (d, J=1 Hz, 1H), 9.01 (d, J=2 Hz, 1H), 8.88 (d, J=2 Hz, 1H), 8.54 (d, J=8 Hz, 1H), 3.73-3.84 (m, 1H), 2.58-2.72 (m, 1H), 2.21-2.36 (m, 1H), 1.90-2.07 (m, 4H), 1.21-1.50 (m, 8H). MS: m/z 330 (M+H). The compound was used as such in the next reaction.

### Intermediate 9

### Methyl 2-(tert-butyl)thiazolo[4,5-b]pyridine-6-carboxylate

[chemical structure]

### A. Methyl 5-bromo-6-pivalamidonicotinate

[chemical structure]

To a stirred solution of methyl 6-amino-5-bromonicotinate (1 g, 4.33 mmol) in pyridine (10 mL), at rt, was added pivaloyl chloride (2.66 mL, 21.64 mmol), followed by a catalytic amount of DMAP (100 mg). The reaction mixture was then heated at 60° C. for 48 h. Upon cooling, the reaction mixture was concentrated under vacuum. The residue was purified by silica gel chromatography, eluting with 0-70% EtOAc:hexanes gradient to give methyl 5-bromo-6-pivalamidonicotinate (1.0 g, 3.01 mmol, 70% yield). ¹H NMR (400 MHz, CD3SOCD₃) δ: 10.03 (s, 1H), 8.93 (d, J=2 Hz, 1H), 8.49 (d, J=2 Hz, 1H), 3.90 (s, 3H), 1.25 (s, 9H). MS: m/z 315/317 (M+H) for Br isotopes.

### B. Methyl 5-bromo-6-(2,2-dimethylpropanethioamido)nicotinate

[chemical structure]

A mixture of methyl 5-bromo-6-pivalamidonicotinate (0.90 g, 2.86 mmol) and Lawesson's reagent (1.386 g, 3.43 mmol) in THF (20 mL) was heated in a sealed tube at 70° C. After 12 h, additional Lawesson's reagent (0.347 mg, 0.858 mmol) was added, and heating was continued for another 4 h. Upon cooling, the reaction mixture was concentrated under vacuum. The residue was purified by silica gel chromatography, eluting with 0-70% EtOAc:hexanes gradient to give methyl 5-bromo-6-(2,2-dimethylpropanethioamido)nicotinate (0.650 g, 1.825 mmol, 64% yield). ¹H NMR (400 MHz, CD3SOCD₃) δ: 11.29 (s, 1H), 9.02 (d, J=2 Hz, 1H), 8.56 (d, J=2 Hz, 1H), 3.92 (s, 3H), 1.40 (s, 9H). MS: m/z 331/333 (M+H) for Br isotopes.

### C. Methyl 2-(tert-butyl)thiazolo[4,5-b]pyridine-6-carboxylate

[chemical structure]

To a stirred solution of methyl 5-bromo-6-(2,2-dimethylpropanethioamido)nicotinate (0.620 g, 1.872 mmol) in DMSO (6.2 mL), at rt, was added sodium hydride (0.082 g, 2.059 mmol). The mixture was then heated in a sealed tube at 70° C. for 10 h. Upon cooling, the reaction mixture was poured into 1N HCl (100 mL) and extracted with EtOAc (2×100 mL). The combined EtOAc layers were washed with water (100 mL) and brine (100 mL), dried over Na₂SO₄, and concentrated under vacuum. The residue was purified by silica gel chromatography, eluting with 0-70% EtOAc:hexanes gradient to give methyl 2-(tert-butyl)thiazolo[4,5-b]pyridine-6-carboxylate (0.320 g, 1.214 mmol, 65% yield). ¹H NMR (400 MHz, CD3SOCD₃) δ: 9.18 (d, J=2 Hz, 1H), 9.15 (d, J=2 Hz, 1H), 3.94 (s, 3H), 1.50 (s, 9H). MS: m/z 251 (M+H).

### Intermediate 10

### (Racemic)-1-((trans)-4-Aminocyclohexyl)-2-(methylsulfonyl)ethan-1-ol trifluoroacetate

[chemical structure]

### A. tert-Butyl ((trans)-4-formylcyclohexyl)carbamate

[chemical structure]

A solution of methyl (trans)-4-((tert-butoxycarbonyl)amino)cyclohexane-1-carboxylate (2 g, 7.77 mmol) in toluene (15 mL), under N₂, was cooled at −78° C. in a dry ice/acetone bath. After stirring for ˜10 min, diisobutylaluminum hydride (1.2 M in toluene, 12.95 mL, 15.54 mmol) was added slowly over ˜10 min. The mixture was stirred in the cold bath for ˜1 h. While in the cold bath, the reaction was quenched by slow, careful addition of a mixture of MeOH (10 mL) and toluene (10 mL). After stirring for ˜10 min, the cold bath was removed and aq. satd. potassium sodium tartrate (˜50 mL) was added slowly. The mixture was stirred at rt for ˜10 min and then it was extracted with Et₂O (separation of the phases was problematic because of heavy emulsions). Additional solid potassium sodium tartrate was added, as well as additional water and Et₂O. Due to heavy emulsions, the biphasic mixture was allowed to stand overnight, which resulted in a clear phase separation. The aqueous phase was extracted 2× with Et₂O. The Et₂O phases were combined, washed with brine, dried over Na₂SO₄, filtered, and concentrated. The residue was purified by silica gel chromatography, eluting with 0-40% EtOAc:hexanes gradient to give tert-butyl ((trans)-4-formylcyclohexyl)carbamate (1.64 g, 6.85 mmol, 88% yield) as a white solid. ¹H NMR (400 MHz, CD₃SOCD₃) δ ppm 9.55 (s, 1H), 6.79 (d, J=8 Hz, 1H), 3.05-3.24 (m, 1H), 2.08-2.26 (m, 1H), 1.72-1.96 (m, 4H), 1.38 (s, 9H), 1.08-1.29 (m, 4H).

### B. tert-Butyl ((trans)-4-vinylcyclohexyl)carbamate

[chemical structure]

To an ice-cold suspension of methyltriphenylphosphonium bromide (5.03 g, 14.08 mmol) in THF (25 mL), under N₂, was added potassium tert-butoxide (1.580 g, 14.08 mmol) in portions. After 5 min, the ice bath was removed and the mixture was stirred at rt. After ˜1 h, tert-butyl ((trans)-4-formylcyclohexyl)carbamate (1.6 g, 7.04 mmol) was added. The mixture was stirred at rt for ˜1 h, and then, aq. satd. NH₄Cl solution (˜10 mL) was slowly added. The mixture was partitioned between EtOAc and water. The aqueous phase was washed 1× with EtOAc. The combined organic phases were washed 1× with brine, dried over Na₂SO₄, filtered, and concentrated. The residue was purified by silica gel chromatography, eluting with 0-50% EtOAc:hexanes gradient (fractions were checked with PMA stain) to give tert-butyl ((trans)-4-vinylcyclohexyl)carbamate (1.329 g, 5.60 mmol, 80% yield) as a white solid. ¹H NMR (400 MHz, CD₃SOCD₃) δ ppm 6.72 (d, J=8 Hz, 1H), 5.66-5.86 (m, 1H), 4.97 (dd, J=17, 2 Hz, 1H), 4.89 (dd, J=10, 2 Hz, 1H), 3.05-3.22 (m, 1H), 1.74-1.94 (m, 3H), 1.65-1.72 (m, 2H), 1.38 (s, 9H), 1.00-1.27 (m, 4H).

### C. Racemic tert-butyl ((trans)-4-(oxiran-2-yl)cyclohexyl)carbamate

[chemical structure]

To an ice-cold solution of tert-butyl ((trans)-4-vinylcyclohexyl)carbamate (1.1 g, 4.88 mmol) in CH₂Cl₂ (20 mL) was added mCPBA (2.246 g, 9.76 mmol) in one portion. After ˜5 min, the ice bath was removed, and the heterogeneous mixture was stirred at rt. After ˜3 h, the reaction mixture was partially concentrated in a rotary evaporator. The residue was partitioned between EtOAc and aq. saturated K₂CO₃ solution. The organic phase was washed 2× with satd. K₂CO₃ solution and 1× with brine, dried over Na₂SO₄, filtered, and concentrated. The residue was purified by silica gel chromatography, eluting with 0-30% EtOAc:hexanes gradient (fractions checked with PMA stain) to give racemic tert-butyl ((trans)-4-(oxiran-2-yl)cyclohexyl)carbamate (1.217 g, 4.64 mmol, 95% yield). ¹H NMR (400 MHz, CD₃SOCD₃) δ ppm 6.73 (d, J=8 Hz, 1H), 3.07-3.24 (m, 1H), 2.65-2.70 (m, 1H), 2.63 (t, J=4 Hz, 1H), 2.47-2.50 (m, 1H), 1.71-1.87 (m, 3H), 1.58-1.69 (m, 1H), 1.38 (s, 9H), 0.96-1.18 (m, 5H).

### D. Racemic tert-butyl ((trans)-4-(1-hydroxy-2-(methylthio)ethyl)cyclohexyl)carbamate

[chemical structure]

To an ice-cold solution of racemic tert-butyl ((trans)-4-(oxiran-2-yl)cyclohexyl)carbamate (500 mg, 1.87 mmol) in DMF (5 mL) was added sodium thiomethoxide (170 mg, 2.42 mmol) in one portion. After a couple of minutes, the cold bath was removed, and the mixture was stirred at rt. After ˜4 h, the reaction mixture was treated with aq. satd. NH₄Cl (˜10 mL), and then partitioned between EtOAc and water. The aqueous phase was extracted 1× with EtOAc. The combined organic phases were washed 1× with brine, dried over Na₂SO₄, filtered, and concentrated. The residue was purified by silica gel chromatography, eluting with 0-60% EtOAc:hexanes gradient (fractions checked with PMA stain) to give racemic tert-butyl ((trans)-4-(1-hydroxy-2-(methylthio)ethyl)cyclohexyl)carbamate (45 mg, 0.148 mmol, 8% yield) as a white solid. ¹H NMR (400 MHz, CD3SOCD₃) δ ppm 6.68 (br d, J=8 Hz, 1H), 4.60 (br s, 1H), 3.36-3.42 (m, 1H; overlapping with water peak), 3.04-3.18 (m, 1H), 2.40-2.60 (m, 2H; overlapping with DMSO solvent peak), 2.06 (s, 3H), 1.68-1.83 (m, 3H), 1.55-1.60 (m, 1H), 1.37 (s, 9H), 1.23-1.31 (m, 1H), 0.97-1.20 (m, 4H).

### E. Racemic tert-butyl ((trans)-4-(1-hydroxy-2-(methylsulfonyl)ethyl)cyclohexyl)carbamate

[chemical structure]

To a solution of racemic tert-butyl ((trans)-4-(1-hydroxy-2-(methylthio)ethyl)cyclohexyl)carbamate (43 mg, 0.149 mmol) in MeOH (5 mL) was added a solution of Oxone® (250 mg, 0.407 mmol) in water (1 mL). After stirring at rt for ˜2.5 h, additional Oxone® (180 mg, 0.293 mmol) dissolved in water (1 mL) was added and the mixture was stirred for another ˜3 h. The reaction mixture was partitioned between EtOAc and water. The organic phase was washed 1× with brine, dried over Na₂SO₄, filtered, and concentrated. The residue was purified by silica gel chromatography, eluting with 10-70% EtOAc:hexanes gradient (fractions checked with PMA stain) to give racemic tert-butyl ((trans)-4-(1-hydroxy-2-(methylsulfonyl)ethyl)cyclohexyl)carbamate (42 mg, 0.118 mmol, 79% yield). ¹H NMR (400 MHz, CD₃OD) δ ppm 4.03-4.16 (m, 1H), 3.89-4.02 (m, 1H), 3.27-3.36 (m, 1H), 3.08-3.18 (m, 1H), 3.05 (d, J=1 Hz, 3H), 1.89-1.99 (m, 1H), 1.64-1.88 (m, 4H), 1.51 (s, 9H), 1.19-1.47 (m, 4H).

### F. Racemic 1-((trans)-4-Aminocyclohexyl)-2-(methylsulfonyl)ethan-1-ol trifluoroacetate

[chemical structure]

To a solution of racemic tert-butyl ((trans)-4-(1-hydroxy-2-(methylsulfonyl)ethyl)cyclohexyl)carbamate (42 mg, 0.131 mmol) in CH₂Cl₂ (4 mL) was added TFA (2 mL). After stirring at rt for ˜2.5 h, the reaction mixture was concentrated in a rotary evaporator. The residue was dissolved in CH₂Cl₂ and concentrated again. Repeated once more and then dried under high vacuum overnight to give crude racemic 1-((trans)-4-aminocyclohexyl)-2-(methylsulfonyl)ethan-1-ol trifluoroacetate (77 mg, 0.137 mmol, ˜100% yield, ˜80% purity by ¹H NMR). ¹H NMR (400 MHz, CD₃OD) δ ppm 3.98 (ddd, J=10, 5, 2 Hz, 1H), 3.29-3.37 (m, 1H; overlapping with MeOH solvent peak), 3.09-3.17 (m, 1H), 3.04-3.06 (m, 1H), 3.00-3.09 (s, 3H), 2.06-2.16 (m, 2H), 1.91-1.99 (m, 1H), 1.81-1.90 (m, 1H), 1.18-1.55 (m, 5H).

### Intermediate 11

### 2-(Azetidin-1-yl)thiazolo[4,5-b]pyridine-6-carboxylic acid

[chemical structure]

### A. 6-Bromo-2-chlorothiazolo[4,5-b]pyridine

[chemical structure]

1^{st} Step:

A mixture of 3,5-dibromopyridin-2-amine (2 g, 7.94 mmol) and potassium O-ethyl carbonodithioate (3.05 g, 19.05 mmol) in DMF (8 mL) was heated in a sealed tube at 130° C. for a total of ˜15 h. After cooling, 1N HCl (˜60 mL) was added slowly. A yellow solid precipitated out. The mixture was stirred at rt for ˜1 h, and then the solid was collected by filtration, washed with water and dried under high vacuum for ˜60 h to give the crude intermediate 6-bromothiazolo[4,5-b]pyridine-2(3H)-thione, which was used as such in the subsequent step.

2^{nd} step:

To a suspension of the crude product from step 1 in CH₂Cl₂ (10 mL) was slowly added sulfuryl chloride (6.45 mL, 79 mmol). The mixture was stirred heavily at rt. After ˜4 h, additional sulfuryl chloride (3.23 mL, 39.7 mmol) was added. The reaction mixture was stirred at rt for a few hours, and then stored in the freezer overnight. Afterwards, the reaction mixture was cooled in an ice bath and treated slowly (very exothermic!!) with water, to decompose excess SO₂Cl₂. The heterogeneous reaction mixture was stirred in the cold bath for a few minutes. The solids were then collected by filtration, washed sequentially with water (˜50 mL) and with CH₂Cl₂ (˜20 mL), and dried by air suction for ˜1 h to give crude 6-bromo-2-chlorothiazolo[4,5-b]pyridine (1.64 g, 6.24 mmol, 79% yield). ¹H NMR (400 MHz, CD3SOCD₃) δ ppm 8.91 (d, J=2 Hz, 1H), 8.82 (d, J=2 Hz, 1H). MS: m/z 249/251 (M+H) for Br isotopes.

### B. 2-(Azetidin-1-yl)-6-bromothiazolo[4,5-b]pyridine

[chemical structure]

To a mixture of 6-bromo-2-chlorothiazolo[4,5-b]pyridine (100 mg, 0.401 mmol) and azetidine hydrochloride (75.0 mg, 0.802 mmol) in DMSO (2 mL) was added cesium carbonate (522 mg, 1.603 mmol). The mixture was stirred at rt for ˜5 minutes, and then it was heated in a sealed tube at 100° C. for 6 h. Upon cooling, the reaction mixture was diluted with water and extracted with EtOAc. The aqueous phase was extracted 4× with EtOAc. The aqueous phase, which contains some undissolved solids, was also extracted 2× with CH₂Cl₂. The EtOAc washes were combined, washed with satd. brine, dried over Na₂SO₄ and filtered. The CH₂Cl₂ washes were combined, washed with satd. brine, dried over Na₂SO₄ and filtered. The EtOAc and CH₂Cl₂ washes were combined and concentrated under vacuo. The residue was purified by silica gel chromatography, eluting with 5-70% (3:1 EtOAc:EtOH):hexanes gradient to give 2-(azetidin-1-yl)-6-bromothiazolo[4,5-b]pyridine (65 mg, 0.229 mmol, 57% yield). ¹H NMR (400 MHz, CD3SOCD₃) δ ppm 8.46 (d, J=2 Hz, 1H), 8.36 (d, J=2 Hz, 1H), 4.14-4.25 (m, 4H), 2.43-2.50 (m, 2H). MS: m/z 270/272 (M+H) for Br isotopes.

### C. Ethyl 2-(azetidin-1-yl)thiazolo[4,5-b]pyridine-6-carboxylate

[chemical structure]

A mixture of 2-(azetidin-1-yl)-6-bromothiazolo[4,5-b]pyridine (60 mg, 0.222 mmol), PdCl₂dppf⋅CH₂Cl₂ (36 mg, 0.044 mmol) and DIEA (0.194 mL, 1.111 mmol) in EtOH (5 mL) was purged with N₂ for a couple of minutes, followed by purging with carbon monoxide for ˜5 min. The reaction mixture was then heated at 80° C. in a sealed tube under a carbon monoxide balloon, for ˜15 h. Upon cooling, the reaction mixture was filtered, and the filtrate was concentrated to dryness. The residue was dissolved in EtOAc with a minimal amount of MeOH and washed 2× with water and 1× with brine. The combined aqueous phases were back-extracted 1× with EtOAc. This EtOAc phase was washed 1× with brine. The EtOAc phases were combined, dried over Na₂SO₄, filtered, and concentrated. The residue was purified by silica gel chromatography, eluting with 5-70% (3:1 EtOAc:EtOH):hexanes gradient to give ethyl 2-(azetidin-1-yl)thiazolo[4,5-b]pyridine-6-carboxylate (42 mg, 0.152 mmol, 68% yield) as a beige solid. ¹H NMR (400 MHz, CD3SOCD₃) δ ppm 8.83 (d, J=2 Hz, 1H), 8.71 (d, J=2 Hz, 1H), 4.33 (q, J=7 Hz, 2H), 4.24 (t, J=7 Hz, 4H), 2.47-2.50 (m, 2H, overlaps with DMSO peak), 1.33 (t, J=7 Hz, 3H). MS: m/z 264 (M+H).

### D. 2-(Azetidin-1-yl)thiazolo[4,5-b]pyridine-6-carboxylic acid

[chemical structure]

To a solution of ethyl 2-(azetidin-1-yl)thiazolo[4,5-b]pyridine-6-carboxylate (40 mg, 0.152 mmol) in 1:1 THF:MeOH (6 mL) was added a solution of NaOH (122 mg, 3.04 mmol) in water (3 mL). The reaction mixture was stirred at rt for ˜2 h, and then it was concentrated in a rotary evaporator. When just 1 or 2 mL of water were leftover, a lot of solids were present. The mixture was diluted with ˜7 mL of water, and it became homogeneous. The solution was then acidified with 1N HCl and washed 2× with EtOAc (not much product is present in the EtOAc washes). During the work-up, the aqueous phase showed some crystalline solids suspended. These solids were collected by filtration, washed sequentially with a minimal amount of water and with hexanes, and then dried under high vacuum overnight to give the first batch of 2-(azetidin-1-yl)thiazolo[4,5-b]pyridine-6-carboxylic acid (14 mg, 0.057 mmol, 37% yield). ¹H NMR (400 MHz, CD₃SOCD₃) δ ppm 8.82 (d, J=2 Hz, 1H), 8.79 (d, J=2 Hz, 1H), 4.29 (t, J=8 Hz, 4H), 2.47-2.57 (m, 2H; overlaps with DMSO solvent peak). MS: m/z 236 (M+H). LCMS of the aqueous phase showed a lot of product present, so the aqueous phase was concentrated to almost dryness in a rotary evaporator. The residual solids were dissolved and concentrated a couple of times with MeOH and a couple of times with toluene, and then they were dried under high vacuum overnight to give a second batch of crude 2-(azetidin-1-yl)thiazolo[4,5-b]pyridine-6-carboxylic acid (21 mg, 0.090 mmol, 59% yield).

### Intermediate 12

### Methyl 2-isopropylthiazolo[4,5-b]pyridine-6-carboxylate

[chemical structure]

### A. Methyl 5-bromo-6-isobutyramidonicotinate

[chemical structure]

To a stirred solution of methyl 6-amino-5-bromonicotinate (1 g, 4.33 mmol) in pyridine (10 mL) was added isobutyryl chloride (0.544 mL, 5.19 mmol). After stirring at rt for ˜2 h, additional isobutyryl chloride (0.544 mL, 5.19 mmol) was added, and stirring was continued for another 2 h. The reaction mixture was then heated at 60° C. overnight. Upon cooling, the reaction mixture was concentrated under vacuum. The residue was purified by silica gel chromatography, eluting with 0-70% EtOAc:hexanes gradient to give methyl 5-bromo-6-isobutyramidonicotinate (0.850 g, 2.68 mmol, 62% yield). ¹H NMR (400 MHz, CD₃SOCD₃) δ: 10.40 (s, 1H), 8.91 (d, J=2 Hz, 1H), 8.48 (d, J=2 Hz, 1H), 3.90 (s, 3H), 2.71 (sept, J=7 Hz, 1H), 1.13 (d, J=7 Hz, 6H). MS: m/z 301/303 (M+H) for Br isotopes.

### B. Methyl 5-bromo-6-(2-methylpropanethioamido)nicotinate

[chemical structure]

A mixture of methyl 5-bromo-6-isobutyramidonicotinate (0.830 g, 2.76 mmol) and Lawesson's reagent (1.338 g, 3.31 mmol) in THF (16 mL) was heated in a sealed tube at 70° C. After 12 h, additional Lawesson's reagent (0.335 g, 0.828 mmol) was added, and the mixture was heated at 70° C. for another 4 h. Upon cooling, the reaction mixture was concentrated under vacuum. The residue was purified by silica gel chromatography, eluting with 0-70% EtOAc:hexanes gradient to give methyl 5-bromo-6-(2-methylpropanethioamido)nicotinate (0.70 g, 1.986 mmol, 72% yield). ¹H NMR (400 MHz, CD3SOCD₃) δ: 11.92 (s, 1H), 9.01 (d, J=2 Hz, 1H), 8.57 (d, J=2 Hz, 1H), 3.91 (s, 3H), 3.13 (sept, J=7 Hz, 1H), 1.25 (d, J=7 Hz, 6H). MS: m/z 317/319 (M+H) for Br isotopes.

### C. Methyl 2-isopropylthiazolo[4,5-b]pyridine-6-carboxylate

[chemical structure]

To a solution of methyl 5-bromo-6-(2-methylpropanethioamido)nicotinate (0.60 g, 1.892 mmol) in DMSO (6 mL), at rt, was added sodium hydride (0.091 g, 2.270 mmol). The mixture was then heated in a sealed tube at 70° C. for 10 h. Upon cooling, the reaction mixture was poured into 1N HCl (50 mL) and extracted with EtOAc (3×50 mL). The combined EtOAc layers were washed with brine, dried over Na₂SO₄, and concentrated under vacuum. The residue was purified by silica gel chromatography, eluting with 0-70% EtOAc:hexanes gradient to give methyl 2-isopropylthiazolo[4,5-b]pyridine-6-carboxylate (0.150 g, 0.603 mmol, 32% yield). ¹H NMR (400 MHz, CD3SOCD₃) δ: 9.18 (d, J=2 Hz, 1H), 9.15 (d, J=2 Hz, 1H), 3.93 (s, 3H), 3.52 (sept, J=7 Hz, 1H), 1.45 (d, J=7 Hz, 6H). MS: m/z 237 (M+H).

### Intermediate 13

### 2-(Pyrrolidin-1-yl)thiazolo[4,5-b]pyridine-6-carboxylic acid

[chemical structure]

### A. 6-Bromo-2-(pyrrolidin-1-yl)thiazolo[4,5-b]pyridine

[chemical structure]

A mixture of 6-bromo-2-chlorothiazolo[4,5-b]pyridine (100 mg, 0.401 mmol, Intermediate 11A), pyrrolidine (0.133 mL, 1.603 mmol) and potassium carbonate (111 mg, 0.802 mmol) in DMSO (1 mL) was heated in a sealed tube at 110° C. for 4 h, and then it stood at rt overnight. Water (˜7 mL) was added slowly to the stirring reaction mixture, and stirring was continued for 10 min. The undissolved solids were collected by filtration, washed sequentially with water and with hexanes, and dried under high vacuum to give 6-bromo-2-(pyrrolidin-1-yl)thiazolo[4,5-b]pyridine (85 mg, 0.284 mmol, 71% yield) as a white solid. ¹H NMR (400 MHz, CD₃SOCD₃) δ ppm 8.45 (d, J=2 Hz, 1H), 8.34 (d, J=2 Hz, 1H), 3.39-3.73 (m, 4H), 1.97-2.10 (m, 4H). MS: m/z 284/286 (M+H) for Br isotopes.

### B. Ethyl 2-(pyrrolidin-1-yl)thiazolo[4,5-b]pyridine-6-carboxylate

[chemical structure]

A mixture of 6-bromo-2-(pyrrolidin-1-yl)thiazolo[4,5-b]pyridine (82 mg, 0.289 mmol), PdCl₂dppf⋅CH₂Cl₂ (47.1 mg, 0.058 mmol) and DIEA (0.252 mL, 1.443 mmol) in EtOH (5 mL) was purged with N₂ for a couple of minutes, followed by purging with carbon monoxide for ˜5 min. The reaction mixture was then heated at 80° C. in a sealed tube under a carbon monoxide balloon for ˜15 h. Upon cooling, the reaction mixture was filtered, and the filtrate was concentrated to dryness. The residue was dissolved in EtOAc with a minimal amount of MeOH and washed 2× with water and 1× with brine. The combined aqueous phases were back extracted 1× with EtOAc. This EtOAc phase was washed 1× with brine. The EtOAc phases were combined, dried over Na₂SO₄, filtered, and concentrated. The residue was purified by silica gel chromatography, eluting with 5-60% (3:1 EtOAc:EtOH):hexanes gradient to give ethyl 2-(pyrrolidin-1-yl)thiazolo[4,5-b]pyridine-6-carboxylate (57 mg, 0.195 mmol, 68% yield) as a beige solid. ¹H NMR (400 MHz, CD3SOCD₃) δ ppm 8.83 (d, J=2 Hz, 1H), 8.69 (d, J=2 Hz, 1H), 4.33 (q, J=7 Hz, 2H), 3.38-3.84 (m, 4H), 2.05 (br s, 4H), 1.34 (t, J=7 Hz, 3H). MS: m/z 278 (M+H).

### C. 2-(Pyrrolidin-1-yl)thiazolo[4,5-b]pyridine-6-carboxylic acid

[chemical structure]

To a solution of ethyl 2-(pyrrolidin-1-yl)thiazolo[4,5-b]pyridine-6-carboxylate (55 mg, 0.198 mmol) in 1:1 THF:MeOH (6 mL) was added a solution of NaOH (229 mg, 5.73 mmol) in water (3 mL). After stirring at rt for ˜2 h, the reaction mixture was acidified to pH˜3 with 6N HCl, and then it was concentrated to dryness in a rotary evaporator. The residue was taken up in MeOH and concentrated again. Repeated twice. The residue was then taken up in CH₂Cl₂ and concentrated. Repeated once, and then dried under high vacuum overnight to give crude 2-(pyrrolidin-1-yl)thiazolo[4,5-b]pyridine-6-carboxylic acid (50 mg, 0.191 mmol, 96% yield). MS: m/z 250 (M+H).

### Intermediate 14

### (S)-2-(2-Methylazetidin-1-yl)thiazolo[4,5-b]pyridine-6-carboxylic acid

[chemical structure]

### A. (S)-6-Bromo-2-(2-methylazetidin-1-yl)thiazolo[4,5-b]pyridine

[chemical structure]

A mixture of 6-bromo-2-chlorothiazolo[4,5-b]pyridine (100 mg, 0.401 mmol, Intermediate 11A), (S)-2-methylazetidine, (1R)-10-camphorsulphonate salt (121 mg, 0.401 mmol) and cesium carbonate (392 mg, 1.202 mmol) in DMSO (1 mL) was heated in a sealed tube at 120° C. for ˜1 h. Upon cooling, the reaction mixture was partitioned between EtOAc and aq. K₂CO₃ solution. The organic phase was washed 1× with satd. K₂CO₃ solution and 1× with brine. The aqueous phases were combined and back-extracted 1× with EtOAc. This EtOAc phase was washed 1× with brine. The EtOAc phases were combined, dried over Na₂SO₄, filtered, and concentrated. The residue was purified by silica gel chromatography, eluting with 0-50% (3:1 EtOAc:EtOH):hexanes gradient to give (S)-6-bromo-2-(2-methylazetidin-1-yl)thiazolo[4,5-b]pyridine (89 mg, 0.298 mmol, 74% yield). ¹H NMR (400 MHz, CD₃SOCD₃) δ ppm 8.46 (d, J=2 Hz, 1H), 8.36 (d, J=2 Hz, 1H), 4.50-4.67 (m, 1H), 4.08-4.16 (m, 1H), 4.00-4.08 (m, 1H), 2.54-2.65 (m, 1H), 2.06-2.18 (m, 1H), 1.50 (d, J=6 Hz, 3H). MS: m/z 284/286 (M+H) for Br isotopes.

### B. Ethyl (S)-2-(2-methylazetidin-1-yl)thiazolo[4,5-b]pyridine-6-carboxylate

[chemical structure]

A mixture of (S)-6-bromo-2-(2-methylazetidin-1-yl)thiazolo[4,5-b]pyridine (86 mg, 0.303 mmol), PdCl₂dppf⋅CH₂Cl₂ (37 mg, 0.045 mmol) and DIEA (0.264 mL, 1.513 mmol) in EtOH (5 mL) was purged with N₂ for a couple of minutes, followed by purging with carbon monoxide for ˜5 min. The reaction mixture was then heated at 80° C. in a sealed tube, under a carbon monoxide balloon, for ˜15 h. Upon cooling, the reaction mixture was filtered, and the filtrate was concentrated to dryness. The residue was dissolved in EtOAc with a minimal amount of MeOH and washed 2× with water and 1× with brine. The combined aqueous phases were back-extracted 1× with EtOAc. This EtOAc phase was washed 1× with brine. The EtOAc phases were combined, dried over Na₂SO₄, filtered, and concentrated. The residue was purified by silica gel chromatography, eluting with 5-70% (3:1 EtOAc:EtOH):hexanes gradient to give ethyl (S)-2-(2-methylazetidin-1-yl)thiazolo[4,5-b]pyridine-6-carboxylate (64 mg, 0.219 mmol, 72% yield). ¹H NMR (400 MHz, CD₃SOCD₃) δ ppm 8.83 (d, J=2 Hz, 1H), 8.70 (d, J=2 Hz, 1H), 4.57-4.72 (m, 1H), 4.33 (q, J=7 Hz, 2H), 4.14-4.23 (m, 1H), 4.03-4.13 (m, 1H), 2.57-2.68 (m, 1H), 2.07-2.20 (m, 1H), 1.53 (d, J=6 Hz, 3H), 1.34 (t, J=7 Hz, 3H). MS: m/z 278 (M+H).

### C. (S)-2-(2-Methylazetidin-1-yl)thiazolo[4,5-b]pyridine-6-carboxylic acid

[chemical structure]

To a solution of ethyl (S)-2-(2-methylazetidin-1-yl)thiazolo[4,5-b]pyridine-6-carboxylate (60 mg, 0.216 mmol) in 1:1 THF:MeOH (6 mL) was added a solution of NaOH (230 mg, 5.75 mmol) in water (3 mL). After stirring at rt for ˜2 h, the reaction mixture was acidified to pH˜3 with 6N HCl, and then it was concentrated to dryness in a rotary evaporator. The residue was taken up in MeOH and concentrated again. Repeated twice. The residue was then taken up in CH₂Cl₂ and concentrated. Repeated once, and then dried under high vacuum overnight to give crude (S)-2-(2-methylazetidin-1-yl)thiazolo[4,5-b]pyridine-6-carboxylic acid (55 mg, 0.21 mmol, 97% yield). MS: m/z 250 (M+H).

### Intermediate 15

### 2-(Cyclopropyl(methyl)amino)thiazolo[4,5-b]pyridine-6-carboxylic acid

[chemical structure]

### A. 6-Bromo-N-cyclopropyl-N-methylthiazolo[4,5-b]pyridin-2-amine

[chemical structure]

A mixture of 6-bromo-2-chlorothiazolo[4,5-b]pyridine (100 mg, 0.401 mmol, Intermediate 11A), N-methylcyclopropanamine hydrochloride (65 mg, 0.601 mmol) and cesium carbonate (392 mg, 1.202 mmol) in DMSO (1 mL) was heated in a sealed tube at 120° C. for ˜2 h. Upon cooling, water (˜5 mL) was added dropwise to the stirring reaction mixture. A solid precipitated out. The solid was collected by filtration, washed sequentially with water and with hexanes, and dried under high vacuum to give 6-bromo-N-cyclopropyl-N-methylthiazolo[4,5-b]pyridin-2-amine (92 mg, 0.308 mmol, 77% yield). ¹H NMR (400 MHz, CD3SOCD₃) δ ppm 8.48 (d, J=2 Hz, 1H), 8.37 (d, J=2 Hz, 1H), 3.23 (s, 3H), 2.81-2.90 (m, 1H), 0.83-0.99 (m, 4H). MS: m/z 284/286 (M+H) for Br isotopes.

### B. Ethyl 2-(cyclopropyl(methyl)amino)thiazolo[4,5-b]pyridine-6-carboxylate

[chemical structure]

A mixture of 6-bromo-N-cyclopropyl-N-methylthiazolo[4,5-b]pyridin-2-amine (89 mg, 0.313 mmol), PdCl₂dppf⋅CH₂Cl₂ (38.4 mg, 0.047 mmol) and DIEA (0.274 mL, 1.566 mmol) in EtOH (5 mL) was purged with N₂ for a couple of minutes, followed by purging with carbon monoxide for ˜5 min. The reaction mixture was then heated at 80° C. in a sealed tube, under a carbon monoxide balloon for ˜15 h. Upon cooling, the reaction mixture was diluted with EtOAc and filtered. The filtrate was concentrated to dryness in a rotary evaporator. The residue was purified directly (no work-up) by silica gel chromatography, eluting with 0-60% (3:1 EtOAc:EtOH):hexanes gradient to give ethyl 2-(cyclopropyl(methyl)amino)thiazolo[4,5-b]pyridine-6-carboxylate (72 mg, 0.247 mmol, 79% yield). ¹H NMR (400 MHz, CD3SOCD₃) δ ppm 8.86 (d, J=2 Hz, 1H), 8.74 (d, J=2 Hz, 1H), 4.33 (q, J=7 Hz, 2H), 3.27 (s, 3H), 2.87-2.98 (m, 1H), 1.34 (t, J=7 Hz, 3H), 0.89-1.01 (m, 4H). MS: m/z 278 (M+H).

### C. 2-(Cyclopropyl(methyl)amino)thiazolo[4,5-b]pyridine-6-carboxylic acid

[chemical structure]

To a solution of ethyl 2-(cyclopropyl(methyl)amino)thiazolo[4,5-b]pyridine-6-carboxylate (70 mg, 0.252 mmol) in 1:1 THF:MeOH (6 mL) was added a solution of NaOH (240 mg, 6.00 mmol) in water (3 mL). After stirring at rt for ˜2.5 h, the reaction mixture was acidified to pH˜3 with 6N HCl, and then it was concentrated to dryness in a rotary evaporator. The residue was taken up in MeOH and concentrated again. Repeated 4×. The residue was then taken up in CH₂Cl₂ and concentrated. Repeated once, and then dried under high vacuum overnight to give crude 2-(cyclopropyl(methyl)amino)thiazolo[4,5-b]pyridine-6-carboxylic acid (69 mg, 0.25 mmol, 99% yield). MS: m/z 250 (M+H).

### Intermediate 16

### 2-(Dicyclopropylamino)thiazolo[4,5-b]pyridine-6-carboxylic acid

[chemical structure]

### A. 6-Bromo-N,N-dicyclopropylthiazolo[4,5-b]pyridin-2-amine

[chemical structure]

A mixture of 6-bromo-2-chlorothiazolo[4,5-b]pyridine (107 mg, 0.429 mmol, Intermediate 11A), dicyclopropylamine hydrochloride (84 mg, 0.629 mmol) and cesium carbonate (419 mg, 1.287 mmol) in DMSO (1 mL) was heated in a sealed tube at 120° C. for ˜2 h. Upon cooling, the reaction mixture was partitioned between EtOAc and water. The organic phase was washed 1× with water and 1× with brine, dried over Na₂SO₄, filtered, and concentrated. The residue was purified by silica gel chromatography, eluting with 0-50% EtOAc:hexanes gradient to give 6-bromo-N,N-dicyclopropylthiazolo[4,5-b]pyridin-2-amine (105 mg, 0.322 mmol, 75% yield). ¹H NMR (400 MHz, CD3SOCD₃) δ ppm 8.48 (d, J=2 Hz, 1H), 8.37 (d, J=2 Hz, 1H), 2.76-2.86 (m, 2H), 0.86-0.97 (m, 8H). MS: m/z 310/312 (M+H) for Br isotopes.

### B. Ethyl 2-(dicyclopropylamino)thiazolo[4,5-b]pyridine-6-carboxylate

[chemical structure]

A mixture of 6-bromo-N,N-dicyclopropylthiazolo[4,5-b]pyridin-2-amine (104 mg, 0.335 mmol), PdCl₂dppf⋅CH₂Cl₂ (41.1 mg, 0.050 mmol) and DIEA (0.293 mL, 1.676 mmol) in EtOH (5 mL) was purged with N₂ for a couple of minutes, followed by purging with carbon monoxide for ˜2 min. The reaction mixture was then heated at 80° C. in a sealed tube, under a carbon monoxide balloon for ˜15 h. Upon cooling, the reaction mixture was diluted with MeOH and filtered. The filtrate was concentrated to dryness in a rotary evaporator. The product was purified directly (no work-up) by silica gel chromatography, eluting with 0-50% EtOAc:hexanes gradient to give ethyl 2-(dicyclopropylamino)thiazolo[4,5-b]pyridine-6-carboxylate (81 mg, 0.254 mmol, 76% yield). ¹H NMR (400 MHz, CD₃SOCD₃) δ ppm 8.87 (d, J=2 Hz, 1H), 8.75 (d, J=2 Hz, 1H), 4.33 (q, J=7 Hz, 2H), 2.87 (tt, J=7, 4 Hz, 2H), 1.34 (t, J=7 Hz, 3H), 0.86-1.04 (m, 8H). MS: m/z 304 (M+H).

### C. 2-(Dicyclopropylamino)thiazolo[4,5-b]pyridine-6-carboxylic acid

[chemical structure]

To a solution of ethyl 2-(dicyclopropylamino)thiazolo[4,5-b]pyridine-6-carboxylate (81 mg, 0.267 mmol) in 1:1 THF:MeOH (6 mL) was added a solution of NaOH (113 mg, 2.83 mmol) in water (3 mL). After stirring at rt for ˜2.5 h, the reaction mixture was acidified to pH˜3 with 6N HCl, and then it was concentrated to dryness in a rotary evaporator. The residue was taken up in MeOH and concentrated again. Repeated twice. The residue was then taken up in CH₂Cl₂ and concentrated. Repeated once, and then dried under high vacuum overnight to give crude 2-(dicyclopropylamino)thiazolo[4,5-b]pyridine-6-carboxylic acid (82 mg, 0.267 mmol, 100% yield). MS: m/z 276 (M+H).

### Intermediate 17

### 2-(Diisopropylamino)thiazolo[4,5-b]pyridine-6-carboxylic acid

[chemical structure]

### A. 6-Bromo-N,N-diisopropylthiazolo[4,5-b]pyridin-2-amine

[chemical structure]

A mixture of 6-bromo-2-chlorothiazolo[4,5-b]pyridine (100 mg, 0.401 mmol, Intermediate 11A), and diisopropylamine (0.286 mL, 2.004 mmol) in DMSO (1 mL) was heated in a sealed tube at 120° C. for ˜2 h. Upon cooling, additional diisopropylamine (0.286 mL, 2.004 mmol) was added, and the mixture was heated in the sealed tube for another 2 h. Upon cooling, the reaction mixture was partitioned between EtOAc and water. The EtOAc phase was washed 1× with water and 1× with brine, dried over Na₂SO₄, filtered, and concentrated. The residue was purified by silica gel chromatography, eluting with 0-40% EtOAc:hexanes gradient to give 6-bromo-N,N-diisopropylthiazolo[4,5-b]pyridin-2-amine (45 mg, 0.136 mmol, 34% yield). ¹H NMR (400 MHz, CD₃SOCD₃) δ ppm 8.41 (d, J=2 Hz, 1H), 8.31 (d, J=2 Hz, 1H), 3.96 (septet, J=7 Hz, 2H), 1.39 (d, J=7 Hz, 12H). MS: m/z 314/316 (M+H) for Br isotopes.

### B. Ethyl 2-(diisopropylamino)thiazolo[4,5-b]pyridine-6-carboxylate

[chemical structure]

A mixture of 6-bromo-N,N-diisopropylthiazolo[4,5-b]pyridin-2-amine (43 mg, 0.137 mmol), PdCl₂dppf⋅CH₂Cl₂ (16.76 mg, 0.021 mmol) and DIEA (0.119 mL, 0.684 mmol) in EtOH (5 mL) was purged with N₂ for a couple of minutes, followed by purging with carbon monoxide for ˜2 min. The reaction mixture was then heated at 80° C. in a sealed tube under a carbon monoxide balloon for ˜15 h. Upon cooling, the reaction mixture was diluted with MeOH and filtered. The filtrate was concentrated to dryness in a rotary evaporator. The residue was purified directly (no work-up) by silica gel chromatography, eluting with 0-50% EtOAc:hexanes gradient to give ethyl 2-(diisopropylamino)thiazolo[4,5-b]pyridine-6-carboxylate (30 mg, 0.093 mmol, 68% yield). ¹H NMR (400 MHz, CD3SOCD₃) δ ppm 8.81 (d, J=2 Hz, 1H), 8.65 (d, J=2 Hz, 1H), 4.33 (q, J=7 Hz, 2H), 3.93-4.08 (m, 2H), 1.41 (br d, J=7 Hz, 12H), 1.34 (t, J=7 Hz, 3H). MS: m/z 308 (M+H).

### C. 2-(Diisopropylamino)thiazolo[4,5-b]pyridine-6-carboxylic acid

[chemical structure]

To a solution of ethyl 2-(diisopropylamino)thiazolo[4,5-b]pyridine-6-carboxylate (30 mg, 0.098 mmol) in 1:1 THF:MeOH (6 mL) was added a solution of NaOH (103 mg, 2.58 mmol) in water (3 mL). After stirring at rt for ˜2.5 h, the reaction mixture was acidified to pH˜3 with 6N HCl, and then it was concentrated to dryness in a rotary evaporator. The residue was taken up in MeOH and concentrated again. Repeated twice. The residue was then taken up in CH₂Cl₂ and concentrated. Repeated once, and then dried under high vacuum overnight to give crude 2-(diisopropylamino)thiazolo[4,5-b]pyridine-6-carboxylic acid (30 mg, 0.098 mmol, 100% yield). MS: m/z 280 (M+H).

### Intermediate 18

### 2-(tert-Butyl(methyl)amino)thiazolo[4,5-b]pyridine-6-carboxylic acid

[chemical structure]

### A. 6-Bromo-N-(tert-butyl)-N-methylthiazolo[4,5-b]pyridin-2-amine

[chemical structure]

A mixture of 6-bromo-2-chlorothiazolo[4,5-b]pyridine (100 mg, 0.401 mmol, Intermediate 11A), N,2-dimethylpropan-2-amine (0.240 mL, 2.004 mmol) and Cs₂CO₃ (196 mg, 0.601 mmol) in DMSO (1 mL) was heated in a sealed tube at 120° C. for ˜2 h. Upon cooling, the reaction mixture was partitioned between EtOAc and water. The EtOAc phase was washed 1× with water and 1× with brine. The combined aqueous phases were back-extracted 2× with EtOAc. These EtOAc phases were combined and washed with brine. The EtOAc phases were combined, dried over Na₂SO₄, filtered, and concentrated. The residue was purified by silica gel chromatography, eluting with 0-50% EtOAc:hexanes gradient to give 6-bromo-N-(tert-butyl)-N-methylthiazolo[4,5-b]pyridin-2-amine (67 mg, 0.212 mmol, 53% yield). ¹H NMR (400 MHz, CD₃SOCD₃) δ ppm 8.46 (d, J=2 Hz, 1H), 8.35 (d, J=2 Hz, 1H), 3.12 (s, 3H), 1.57 (s, 9H). MS: m/z 300/302 (M+H) for Br isotopes.

### B. Ethyl 2-(tert-butyl(methyl)amino)thiazolo[4,5-b]pyridine-6-carboxylate

[chemical structure]

A mixture of 6-bromo-N-(tert-butyl)-N-methylthiazolo[4,5-b]pyridin-2-amine (65 mg, 0.217 mmol), PdCl₂dppf⋅CH₂Cl₂ (27 mg, 0.032 mmol) and DIEA (0.189 mL, 1.083 mmol) in EtOH (5 mL) was purged with N₂ for a couple of minutes, followed by purging with carbon monoxide for ˜2 min (some material was accidentally spilled during purging). The reaction mixture was then heated at 80° C. in a sealed tube under a carbon monoxide balloon for ˜15 h. Upon cooling, the reaction mixture was diluted with MeOH and filtered. The filtrate was concentrated to dryness in a rotary evaporator. The residue was purified directly (no work-up) by silica gel chromatography, eluting with 0-50% EtOAc:hexanes gradient to give ethyl 2-(tert-butyl(methyl)amino)thiazolo[4,5-b]pyridine-6-carboxylate (26 mg, 0.084 mmol, 39% yield). ¹H NMR (400 MHz, CD₃SOCD₃) δ ppm 8.82 (d, J=2 Hz, 1H), 8.68 (d, J=2 Hz, 1H), 4.31 (q, J=7 Hz, 2H), 3.15 (s, 3H), 1.57 (s, 9H), 1.32 (t, J=7 Hz, 3H). MS: m/z 294 (M+H).

### C. 2-(tert-Butyl(methyl)amino)thiazolo[4,5-b]pyridine-6-carboxylic acid

[chemical structure]

To a solution of ethyl 2-(tert-butyl(methyl)amino)thiazolo[4,5-b]pyridine-6-carboxylate (26 mg, 0.089 mmol) in 1:1 THF:MeOH (6 mL) was added a solution of NaOH (92 mg, 2.30 mmol) in water (3 mL). After stirring at rt for ˜2.5 h, the reaction mixture was concentrated in a rotary evaporator, down to 1 mL of water. The residue was diluted with MeOH and acidified to pH˜3 with 6N HCl. The mixture was concentrated again in a rotary evaporator. The residue was taken up in MeOH and concentrated again. Repeated twice. The residue was then taken up in CH₂Cl₂ and concentrated. Repeated once, and then dried under high vacuum overnight to give crude 2-(tert-butyl(methyl)amino)thiazolo[4,5-b]pyridine-6-carboxylic acid (25 mg, 0.08 mmol, 90% yield). MS: m/z 266 (M+H).

### Intermediate 19

### N-((trans)-4-(2-Hydroxypropan-2-yl)cyclohexyl)-2-thioxo-2,3-dihydrothiazolo[4,5-b]pyridine-6-carboxamide

[chemical structure]

### A. 6-amino-5-bromo-N-((trans)-4-(2-hydroxypropan-2-yl)cyclohexyl)nicotinamide

[chemical structure]

To a suspension of 6-amino-5-bromonicotinic acid (1 g, 4.61 mmol) in DMF (8 mL) was added DIEA (0.926 mL, 5.30 mmol), followed by HATU (1.927 g, 5.07 mmol) in one portion. Initially, the reaction became homogeneous, but after ˜1 min, heavy precipitation occurred. The mixture was stirred for ˜5 min, and 2-((trans)-4-aminocyclohexyl)propan-2-ol (0.797 g, 5.07 mmol) was added, followed by additional DIEA (0.926 mL, 5.30 mmol) (the mixture was mainly homogeneous except for a very small amount of undissolved cyclohexylamine reagent). The reaction mixture was stirred at rt for ˜30 min. Before adding water to crash the product, the reaction mixture was filtered to remove a small amount of solid. After filtration, the filtrate was stirred, and water was added slowly until the mixture became cloudy (˜50 mL). Upon further stirring, a solid precipitated out. The solid was collected by filtration, washed sequentially with water and with hexanes and dried under high vacuum at 60° C. overnight to give 6-amino-5-bromo-N-((trans)-4-(2-hydroxypropan-2-yl)cyclohexyl)nicotinamide (1.32 g, 3.52 mmol, 75% yield) as a light beige solid. ¹H NMR (400 MHz, CD3SOCD₃) δ ppm 8.46 (d, J=2 Hz, 1H), 8.17 (d, J=2 Hz, 1H), 8.01 (d, J=8 Hz, 1H), 6.77 (brs, 2H), 4.05 (s, 1H), 3.57-3.73 (m, 1H), 1.72-1.97 (m, 4H), 1.19-1.32 (m, 2H), 1.05-1.19 (m, 3H), 1.04 (s, 6H). MS: m/z 356/358 (M+H) for Br isotopes.

### B. N-((trans)-4-(2-Hydroxypropan-2-yl)cyclohexyl)-2-thioxo-2,3-dihydrothiazolo[4,5-b]pyridine-6-carboxamide

[chemical structure]

A mixture of 6-amino-5-bromo-N-((trans)-4-(2-hydroxypropan-2-yl)cyclohexyl)nicotinamide (175 mg, 0.491 mmol) and potassium O-ethyl carbonodithioate (236 mg, 1.474 mmol) in DMF (1 mL) was heated at 135° C. for ˜15 h. Upon cooling, the heterogeneous mixture was stirred at rt, diluted with water (˜10 mL) and then acidified by slow, dropwise addition of 1N HCl (˜5 mL). Heavy precipitation occurred. The mixture was stirred for a few minutes, and then the solids were collected by filtration, washed sequentially with water and with hexanes, and dried by air suction for ˜1 h to give N-((trans)-4-(2-hydroxypropan-2-yl)cyclohexyl)-2-thioxo-2,3-dihydrothiazolo[4,5-b]pyridine-6-carboxamide (165 mg, 0.446 mmol, 91% yield) as a dark pink solid. ¹H NMR (400 MHz, CD3SOCD₃) δ ppm 14.50 (br s, 1H), 8.78 (d, J=2 Hz, 1H), 8.50 (d, J=2 Hz, 1H), 8.41 (d, J=8 Hz, 1H), 4.05 (br s, 1H), 3.62-3.79 (m, 1H), 1.87-1.97 (m, 2H), 1.78-1.87 (m, 2H), 1.23-1.36 (m, 2H), 1.07-1.23 (m, 3H), 1.05 (s, 6H). MS: m/z 352 (M+H).

### Intermediate 20

### 2-Chloro-N-((trans)-4-(2-hydroxypropan-2-yl)cyclohexyl)thiazolo[4,5-b]pyridine-6-carboxamide

[chemical structure]

To an ice-cold suspension of N-((trans)-4-(2-hydroxypropan-2-yl)cyclohexyl)-2-thioxo-2,3-dihydrothiazolo[4,5-b]pyridine-6-carboxamide (170 mg, 0.484 mmol) in CH₂Cl₂ (3 mL) was added sulfuryl chloride (0.055 mL, 0.677 mmol) dropwise. After addition, the cold bath was removed, and the mixture was stirred at rt. The reaction mixture remained heterogeneous at all times, although its appearance changed. After ˜45 min, the reaction mixture was cooled in an ice bath, and additional sulfuryl chloride (0.028 mL, 0.339 mmol) was added. After the addition, the ice bath was removed. The reaction mixture was cooled in an ice bath, and water (˜1 mL) was added to quench excess reagent. The heterogeneous mixture was partitioned between EtOAc and satd. NaHCO₃ solution (some solids remained undissolved). The organic phase was washed 1× with brine, dried over Na₂SO₄, filtered, and concentrated. The residue was purified by silica gel chromatography, eluting with 0-50% (3:1 EtOAc:EtOH):hexanes gradient to give 2-chloro-N-((trans)-4-(2-hydroxypropan-2-yl)cyclohexyl)thiazolo[4,5-b]pyridine-6-carboxamide (30 mg, 0.081 mmol, 17% yield) as a white solid. ¹H NMR (400 MHz, CD3SOCD₃) δ ppm 9.08 (d, J=2 Hz, 1H), 8.98 (d, J=2 Hz, 1H), 8.57 (d, J=8 Hz, 1H), 4.06 (s, 1H), 3.65-3.80 (m, 1H), 1.88-1.97 (m, 2H), 1.77-1.88 (m, 2H), 1.24-1.37 (m, 2H), 1.06-1.23 (m, 3H), 1.04 (s, 6H). MS: m/z 354 (M+H).

### Intermediate 21

### Racemic (1S,3S)-5-aminocyclohexane-1,3-diol

[chemical structure]

### A. 5-(Tosyloxy)cyclohexane-1,3-diyl dibenzoate (diastereomeric mixture)

[chemical structure]

To a stirred solution of a cis and trans mixture of cyclohexane-1,3,5-triol (1.5 g, 11.35 mmol) in pyridine (7.5 mL) was added tosyl chloride (2.60 g, 13.62 mmol) at rt. After stirring for ˜24 h, the reaction mixture was concentrated under vacuum to yield a viscous gel. This crude material was dissolved in pyridine (15 mL) and benzoyl chloride (15 mL) was added. The reaction mixture was stirred at rt for ˜24 hours, and then it was concentrated under vacuum. The residue was dissolved in EtOAc (300 mL) and washed with water (2×300 mL) and brine (300 mL). The EtOAc layer was dried over Na₂SO₄, filtered, and concentrated under vacuum. The residue was purified by silica gel chromatography, eluting with 0-70% EtOAc:hexanes gradient over 30 min, followed by 70-100% EtOAc:hexanes over 5 min. The products eluted around 10-20% EtOAc:hexanes gradient. A tri-benzoylated derivative was isolated and discarded. The 5-(tosyloxy)cyclohexane-1,3-diyl dibenzoate was isolated as a mixture of mainly two diastereomers (2.65 g, 4.82 mmol, 42% yield, 90% purity) which was used in the next step without further purification. MS: m/z 495 (M+H).

### B. Racemic (1R,3R)-5-azidocyclohexane-1,3-diyl dibenzoate and (1R,3S,5s)-5-Azidocyclohexane-1,3-diyl dibenzoate

[chemical structure]

To a stirred solution of the crude diastereomeric mixture of 5-(tosyloxy)cyclohexane-1,3-diyl dibenzoate (2.6 g, 4.82 mmol, ˜90% purity), from the previous step, in DMF (26 mL) was added sodium azide (6.84 g, 105 mmol) at rt. The reaction mixture was then heated for 4 hours at 80° C. Upon cooling, the reaction mixture was diluted with EtOAc (300 mL) and washed with water (2×300 mL) and brine (300 mL). The EtOAc layer was dried over Na₂SO₄, filtered, and concentrated under vacuum. The residue was purified by silica gel chromatography, eluting with 0-40% EtOAc:hexanes gradient over 20 min, followed by 40-100% EtOAc:hexanes over 5 min. The products eluted around 15-20% EtOAc:hexanes. The first eluting compound corresponded to the elimination product and it was discarded. The second eluting compound corresponded to racemic (1R,3R)-5-azidocyclohexane-1,3-diyl dibenzoate (550 mg, 1.43 mmol, 27% yield, ˜75% purity by LCMS and ¹H NMR). ¹H NMR (400 MHz, CDCl₃) δ ppm 8.02-8.11 (m, 4H), 7.57-7.65 (m, 2H), 7.45-7.53 (m, 4H), 5.66 (quin, J=3 Hz, 1H), 5.46 (tt, J=11.4 Hz, 1H), 3.94 (tt, J=11.4 Hz, 1H), 2.59-2.70 (m, 1H), 2.42-2.52 (m, 1H), 2.32-2.41 (m, 1H), 1.88 (ddd, J=14, 11.3 Hz, 1H), 1.66-1.77 (m, 2H). MS: m/z 388 (M+Na). The third eluting compound corresponded to the meso isomer (1R,3S,5s)-5-azidocyclohexane-1,3-diyl dibenzoate (1.0 g, 2.60 mmol, 49% yield). ¹H NMR (400 MHz, CDCl₃) δ ppm 7.96 (dd, J=7, 1 Hz, 4H), 7.50-7.56 (m, 2H), 7.29-7.35 (m, 4H), 5.45-5.53 (m, 2H), 4.16-4.25 (m, 1H), 2.14-2.36 (m, 4H), 1.98 (ddd, J=13, 9, 3 Hz, 2H). MS: m/z 388 (M+Na). The relative stereochemistry of the two isomers was determined in the subsequent step, using NMR experiments.

### C. Racemic (1S,3S)-5-azidocyclohexane-1,3-diol

[chemical structure]

To a stirred solution of racemic (1S,3S)-5-azidocyclohexane-1,3-diyl dibenzoate (550 mg, 1.43 mmol, ˜75% purity) in a mixture of methanol (5 mL) and THF (5 mL) was added a solution of NaOH (1204 mg, 30.1 mmol) in water (5 mL). After stirring at rt for ˜18 h, the reaction mixture was concentrated to dryness under vacuum. The residue was diluted with water (30 mL) and extracted with EtOAc (4×30 mL). The combined EtOAc layers were dried over Na₂SO₄, filtered, and concentrated to yield racemic (1S,3S)-5-azidocyclohexane-1,3-diol (200 mg, 1.018 mmol, 68% yield, ˜80% purity by ¹H NMR) as a white solid. ¹H NMR (CD₃OD) δ: 4.24 (quin, J=3 Hz, 1H), 3.94-4.04 (m, 1H), 3.66-3.77 (m, 1H), 2.21-2.30 (m, 1H), 1.97-2.10 (m, 2H), 1.14-1.48 (m, 3H). (The relative stereochemistry was determined by NMR experiments).

### D. Racemic (1S,3S)-5-aminocyclohexane-1,3-diol

[chemical structure]

To a stirred suspension of Pd—C(135 mg, 1.272 mmol) in methanol (1 mL) was added a solution of racemic (1S,3S)-5-azidocyclohexane-1,3-diol (200 mg, 1.272 mmol) in methanol (3 mL). The reaction mixture was stirred at rt under an atmosphere of hydrogen gas. After ˜7 h, the reaction mixture was filtered through a Celite® bed, and the bed was thoroughly washed with methanol. The filtrate was concentrated under vacuum to yield the crude racemic (1S,3S)-5-aminocyclohexane-1,3-diol (160 mg, 0.976 mmol, 77% yield, ˜80% purity by 1H NMR) as a viscous liquid, which was used as such, without further purification. ¹H NMR (CD₃OD) δ: 4.20 (quin, J=3 Hz, 1H), 3.97 (s, 1H), 3.12 (s, 1H), 2.07-2.26 (m, 1H), 1.87-2.06 (m, 2H), 1.02-1.48 (m, 3H).

### EXAMPLES

### Example 1

### 2-Bromo-N-(trans)-4-(2-hydroxypropan-2-yl)cyclohexyl)thieno[3,2-b]pyridine-6-carboxamide

[chemical structure]

To a stirring suspension of 2-bromothieno[3,2-b]pyridine-6-carboxylic acid (75 mg, 0.291 mmol, Intermediate 1) in DMF (2 mL) was added DIEA (0.063 mL, 0.363 mmol), followed by HATU (133 mg, 0.349 mmol) in one portion. After ˜3 min, 2-(trans)-4-aminocyclohexyl)propan-2-ol (57 mg, 0.363 mmol) was added in one portion, followed by additional DIEA (0.063 mL, 0.363 mmol). After ˜20 min, water (˜7 mL) was added slowly, dropwise. A beige solid precipitated out. The solid was collected by filtration, washed sequentially with water (˜30 mL) and with hexanes (˜30 mL), and dried under high vacuum to give 2-bromo-N-(trans)-4-(2-hydroxypropan-2-yl)cyclohexyl)thieno[3,2-b]pyridine-6-carboxamide (102 mg, 0.244 mmol, 84% yield) as a light brown solid. ¹H NMR (400 MHz, CDCl₂) δ ppm 8.97 (d, J=2 Hz, 1H), 8.54 (d, J=2 Hz, 1H), 7.65 (s, 1H), 6.06 (d, J=8 Hz, 1H), 3.99 (br. s., 1H), 2.13-2.35 (m, 2H), 1.86-2.05 (m, 2H), 1.25-1.42 (m, 5H), 1.23 (s, 6H). MS: m/z 397/399 (M+H) for Br isotopes.

### Example 2

### 2-Cyclopropyl-N-(trans)-4-(2-hydroxypropan-2-yl)cyclohexyl)thieno[3,2-b]pyridine-6-carboxamide

[chemical structure]

To a mixture of 2-bromo-N-(trans)-4-(2-hydroxypropan-2-yl)cyclohexyl)thieno[3,2-b]pyridine-6-carboxamide (50 mg, 0.126 mmol, Example 1), cyclopropylboronic acid (32 mg, 0.378 mmol), Pd₂(dba)₃ (7 mg, 7.55 μmol), and 2-dicyclohexylphosphino-2′,6′-dimethoxyphenyl (S-PHOS) (12 mg, 0.030 mmol) in toluene (3 mL) was added 2M Na₂CO₃ (0.189 mL, 0.378 mmol). The mixture was purged with N₂ for about 5 min, and then it was heated at 110° C. in a sealed tube for ˜14 h. Upon cooling, the reaction mixture was diluted with EtOAc and washed 2× with satd. K₂CO₃ solution and 1× with brine, dried over Na₂SO₄, filtered, and concentrated. The residue was purified by silica gel chromatography, eluting with 0-50% (3:1 EtOAc:EtOH):hexanes gradient. The product was repurified by silica gel chromatography, eluting with 0-60% (3:1 EtOAc:EtOH):hexanes gradient to give 2-cyclopropyl-N-(trans)-4-(2-hydroxypropan-2-yl)cyclohexyl)thieno[3,2-b]pyridine-6-carboxamide (23 mg, 0.061 mmol, 48% yield) as a white solid. ¹H NMR (400 MHz, CD₃SOCD₃) δ ppm 8.98 (d, J=2 Hz, 1H), 8.70 (d, J=2 Hz, 1H), 8.39 (d, J=8 Hz, 1H), 7.33 (s, 1H), 4.07 (s, 1H), 3.65-3.81 (m, 1H), 2.29-2.42 (m, 1H), 1.89-1.99 (m, 2H), 1.77-1.88 (m, 2H), 1.24-1.39 (m, 2H), 1.07-1.23 (m, 5H), 1.05 (s, 6H), 0.88-0.96 (m, 2H). MS: m/z 359 (M+H).

### Example 3

### 2-Bromo-N-(cis)-3-hydroxy-3-methylcyclobutyl)thieno[3,2-b]pyridine-6-carboxamide

[chemical structure]

To a stirring suspension of 2-bromothieno[3,2-b]pyridine-6-carboxylic acid (70 mg, 0.271 mmol, Intermediate 1) in DMF (2 mL) was added DIEA (0.059 mL, 0.339 mmol), followed by HATU (124 mg, 0.325 mmol) in one portion. After ˜5 min, DIEA (0.118 mL, 0.678 mmol) was added, followed by cis-3-amino-1-methylcyclobutanol hydrochloride (47 mg, 0.339 mmol). After ˜30 min, water (˜12 mL) was added slowly, dropwise. The mixture got cloudy and it was stirred for ˜15 min. A beige solid precipitated out, and it was collected by filtration, washed sequentially with water (˜20 mL) and with hexanes (˜20 mL), and dried under high vacuum to give 2-bromo-N-(cis)-3-hydroxy-3-methylcyclobutyl)thieno[3,2-b]pyridine-6-carboxamide (75 mg, 0.209 mmol, 77% yield) as a light brown solid. ¹H NMR (400 MHz, CD3SOCD₃) δ ppm 9.05 (d, J=2 Hz, 1H), 8.87 (d, J=7 Hz, 1H), 8.85 (d, J=2 Hz, 1H), 7.88 (s, 1H), 5.02 (s, 1H), 3.90-4.09 (m, 1H), 2.28-2.37 (m, 2H), 2.06-2.20 (m, 2H), 1.28 (s, 3H). MS: m/z 341/343 (M+H) for Br isotopes.

### Example 4

### 2-Cyclopropyl-N-(cis)-3-hydroxy-3-methylcyclobutyl)thieno[3,2-b]pyridine-6-carboxamide

[chemical structure]

To a mixture of 2-bromo-N-(cis)-3-hydroxy-3-methylcyclobutyl)thieno[3,2-b]pyridine-6-carboxamide (37 mg, 0.108 mmol, Example 3), cyclopropylboronic acid (28 mg, 0.325 mmol), Pd₂(dba)₃ (6 mg, 6.51 μmol), and 2-dicyclohexylphosphino-2′,6′-dimethoxybiphenyl (S-PHOS) (11 mg, 0.026 mmol) in toluene (3 mL) was added 2M Na₂CO₃ (0.163 mL, 0.325 mmol). The mixture was purged with N₂ for about 5 min, and then it was heated at 110° C. in a sealed tube for ˜15 h. (The reaction mixture then stood at rt for about 48 h). The reaction mixture was diluted with EtOAc and washed 2× with satd. K₂CO₃ solution and 1× with brine, dried over Na₂SO₄, filtered, and concentrated. The residue was purified by silica gel chromatography, eluting with 5-50% ((3:1) EtOAc:EtOH):hexanes gradient to give 2-cyclopropyl-N-(cis)-3-hydroxy-3-methylcyclobutyl)thieno[3,2-b]pyridine-6-carboxamide (13 mg, 0.041 mmol, 38% yield). ¹H NMR (400 MHz, CD₃OD) δ ppm 8.97 (d, J=2 Hz, 1H), 8.67 (d, J=2 Hz, 1H), 7.26 (s, 1H), 4.13 (quin, J=8 Hz, 1H), 2.46-2.61 (m, 2H), 2.32-2.43 (m, 1H), 2.14-2.27 (m, 2H), 1.42 (s, 3H), 1.21-1.31 (m, 2H), 0.93-1.01 (m, 2H). MS: m/z 303 (M+H).

### Example 5

### N-(trans)-4-(2-Hydroxypropan-2-yl)cyclohexyl)-2-(isopropylamino)thiazolo[4,5-b]pyridine-6-carboxamide

[chemical structure]

To a stirring suspension of 2-(isopropylamino)thiazolo[4,5-b]pyridine-6-carboxylic acid (40 mg, 0.169 mmol, Intermediate 2) in DMF (2 mL) was added DIEA (0.037 mL, 0.211 mmol), followed by HATU (77 mg, 0.202 mmol) in one portion. After ˜2 min, 2-(trans)-4-aminocyclohexyl)propan-2-ol (33 mg, 0.211 mmol) was added in one portion, followed by additional DIEA (0.037 mL, 0.211 mmol). After ˜30 min, water was added, and the mixture was extracted with EtOAc. The EtOAc phase was washed 1× with aq. satd. K₂CO₃ solution and 1× with brine. The combined aqueous phases were back-extracted 2× with EtOAc. The EtOAc washes were combined and washed with brine. All the EtOAc phases were combined, dried over Na₂SO₄, filtered, and concentrated. The residue was purified by silica gel chromatography, eluting with 10-70% ((3:1) EtOAc:EtOH):hexanes gradient to give the product as a white solid. The solid was triturated with a mixture of CH₂Cl₂ and hexanes (˜1:1), and then it was collected by filtration and dried under high vacuum to give N-(trans)-4-(2-hydroxypropan-2-yl)cyclohexyl)-2-(isopropylamino)thiazolo[4,5-b]pyridine-6-carboxamide (37 mg, 0.093 mmol, 55% yield). ¹H NMR (400 MHz, CD3SOCD₃) δ ppm 8.70 (d, J=2 Hz, 1H), 8.64 (br. s., 1H), 8.44 (d, J=2 Hz, 1H), 8.21 (d, J=8 Hz, 1H), 3.97-4.15 (m, 2H), 3.62-3.77 (m, 1H), 1.76-1.98 (m, 4H), 1.25 (d, J=6 Hz, 6H), 1.05 (s, 6H), 0.98-1.37 (m, 5H). MS: m/z 377 (M+H).

### Example 6

### N-(trans)-4-(2-Hydroxypropan-2-yl)cyclohexyl)-2-(isopropyl(methyl)amino)thiazolo[4,5-b]pyridine-6-carboxamide

[chemical structure]

To a stirring solution of N-(trans)-4-(2-hydroxypropan-2-yl)cyclohexyl)-2-(isopropylamino)thiazolo[4,5-b]pyridine-6-carboxamide (17 mg, 0.045 mmol, Example 5) in DMF (2 mL) was added Cs₂CO₃ (24 mg, 0.074 mmol), followed by methyl iodide (9 μl, 0.144 mmol). After ˜3 h, water (˜2 mL) was added, and the mixture was stirred at rt overnight. The mixture was then extracted 1× with EtOAc. The EtOAc phase was washed 1× with brine, dried over Na₂SO₄, filtered, and concentrated. (TLC of the EtOAc phase (3:1 EtOAc:EtOH) shows two spots at R_{f}˜0.9 and ˜0.75). The residue was purified by silica gel chromatography, eluting with 10-100% ((3:1 EtOAc:EtOH):hexanes gradient. The first eluting compound corresponds to the undesired isomer, (E)-N-(trans)-4-(2-hydroxypropan-2-yl)cyclohexyl)-2-(isopropylimino)-3-methyl-2,3-dihydrothiazolo[4,5-b]pyridine-6-carboxamide (6 mg, 0.015 mmol, 32% yield), which was obtained as a white solid. ¹H NMR (400 MHz, CDCl₃) δ ppm 8.45 (s, 1H), 7.97 (s, 1H), 5.84 (d, J=7 Hz, 1H), 3.86-4.01 (m, 1H), 3.54 (s, 3H), 3.24-3.39 (m, 1H), 2.14-2.28 (m, 2H), 1.86-2.03 (m, 2H), 1.26 (d, J=6 Hz, 6H), 1.23-1.40 (m, 6H), 1.22 (s, 6H). MS: m/z 391 (M+H). The more polar compound corresponds to the desired isomer N-(trans)-4-(2-hydroxypropan-2-yl)cyclohexyl)-2-(isopropyl(methyl)amino)thiazolo[4,5-b]pyridine-6-carboxamide (5 mg, 0.012 mmol, 27% yield), which was obtained as a yellow solid. ¹H NMR (400 MHz, CDCl₃) δ ppm 8.72 (d, J=2 Hz, 1H), 8.41 (d, J=2 Hz, 1H), 6.08 (d, J=7 Hz, 1H), 4.20-5.59 (m, 1H), 3.89-4.04 (m, 1H), 3.13 (br. s., 3H), 2.20 (br. s., 2H), 1.96 (br. s., 2H), 1.33 (d, J=7 Hz, 6H), 1.24-1.40 (m, 6H), 1.22 (s, 6H) (The structural assignments of the two isomers were done by NOE experiments).

### Example 7

### N-(trans)-4-(2-Hydroxypropan-2-yl)cyclohexyl)-2-(methylamino)thiazolo[4, 5-b]pyridine-6-carboxamide

[chemical structure]

To a stirring suspension of 2-(methylamino)thiazolo[4,5-b]pyridine-6-carboxylic acid (25 mg, 0.119 mmol, Intermediate 3) in DMF (2 mL) was added DIEA (0.026 mL, 0.149 mmol), followed by HATU (55 mg, 0.143 mmol) in one portion. After ˜3 min, 2-(trans)-4-aminocyclohexyl)propan-2-ol (24 mg, 0.149 mmol) was added in one portion, followed by additional DIEA (0.026 mL, 0.149 mmol). After stirring for ˜1 h, the reaction mixture was diluted with water and extracted with EtOAc. The aqueous phase was extracted 5× with EtOAc, containing 10% MeOH (some product was still present in the aqueous phase) and 2× with CH₂Cl₂ containing 10% MeOH. The organic phases were combined, dried over Na₂SO₄, filtered, and concentrated. The residue was purified by silica gel chromatography, eluting with 10-80% ((3:1) EtOAc:EtOH):hexanes gradient to give N-(trans)-4-(2-hydroxypropan-2-yl)cyclohexyl)-2-(methylamino)thiazolo[4,5-b]pyridine-6-carboxamide (27 mg, 0.074 mmol, 62% yield) as an off-white solid. ¹H NMR (400 MHz, CD3SOCD₃) δ ppm 8.71 (d, J=2 Hz, 1H), 8.63 (br. s., 1H), 8.46 (d, J=2 Hz, 1H), 8.21 (d, J=8 Hz, 1H), 4.06 (s, 1H), 3.62-3.78 (m, 1H), 3.00 (d, J=4 Hz, 3H), 1.87-1.95 (m, 2H), 1.79-1.87 (m, 2H), 1.22-1.36 (m, 2H), 1.05 (s, 6H), 0.92-1.22 (m, 3H). MS: m/z 349 (M+H).

### Example 8

### 2-Cyclopropyl-N-(trans)-4-(2-hydroxypropan-2-yl)cyclohexyl)thiazolo[4,5-b]pyridine-6-carboxamide

[chemical structure]

To a solution of 2-cyclopropylthiazolo[4,5-b]pyridine-6-carboxylic acid (4.51 g, 20.48 mmol, Intermediate 4) and DIEA (4.29 mL, 24.57 mmol) in DMF (20 mL) was added HATU (9.34 g, 24.57 mmol) in portions. After ˜5 minutes, 2-((trans)-4-aminocyclohexyl)propan-2-ol (3.70 g, 23.55 mmol) was added in portions, followed by additional DIEA (4.29 mL, 24.57 mmol). Shortly after the addition, a solid precipitated out. After stirring for ˜20 min, water (˜120 mL) was added slowly. The mixture was stirred for ˜15 min, and then the solids were collected by filtration, washed sequentially with water and with hexanes and dried under high vacuum at 60° C. to give 2-cyclopropyl-N-(trans)-4-(2-hydroxypropan-2-yl)cyclohexyl)thiazolo[4,5-b]pyridine-6-carboxamide (6.68 g, 17.65 mmol, 86% yield) as a yellow solid. ¹H NMR (400 MHz, CD3SOCD₃) δ ppm 8.99 (d, J=2 Hz, 1H), 8.86 (d, J=2 Hz, 1H), 8.45 (d, J=8 Hz, 1H), 4.05 (s, 1H), 3.65-3.78 (m, 1H), 2.57-2.66 (m, 1H), 1.76-2.00 (m, 4H), 1.05-1.36 (m, 9H), 1.03 (s, 6H). MS: m/z 360 (M+H).

### Example 9

### 2-(Dimethylamino)-N-(trans)-4-(2-hydroxypropan-2-yl)cyclohexyl)thiazolo[4,5-b]pyridine-6-carboxamide

[chemical structure]

To a stirring solution of N-(trans)-4-(2-hydroxypropan-2-yl)cyclohexyl)-2-(methylamino)thiazolo[4,5-b]pyridine-6-carboxamide (15 mg, 0.043 mmol, Example 7) in DMF (2 mL) was added Cs₂CO₃ (24 mg, 0.074 mmol), followed by methyl iodide (7 mg, 0.052 mmol). The reaction mixture was stirred at rt overnight, and then it was partitioned between EtOAc and brine. The organic phase was washed 1× with satd. brine. The combined aqueous phases were back-extracted 2× with EtOAc. These EtOAc phases were washed with satd. brine. The organic phases were combined, dried over Na₂SO₄, filtered, and concentrated. TLC (3:1 EtOAc:EtOH) shows a major spot at R_{f}=0.5 and a smaller one at R_{f}=0.7. The material was purified by silica gel chromatography, eluting with 10-100% ((3:1) EtOAc:EtOH):hexanes gradient. The first eluting compound corresponds to the undesired isomer (E)-N-(trans)-4-(2-hydroxypropan-2-yl)cyclohexyl)-3-methyl-2-(methylimino)-2,3-dihydrothiazolo[4,5-b]pyridine-6-carboxamide (3.5 mg, 0.0098 mmol, 21% yield). ¹H NMR (400 MHz, CD₃OD) δ ppm 8.63 (d, J=2 Hz, 1H), 8.19 (d, J=2 Hz, 1H), 3.83 (tt, J=12, 4 Hz, 1H), 3.53 (s, 3H), 3.14 (s, 3H), 2.04-2.13 (m, 2H), 1.93-2.01 (m, 2H), 1.30-1.46 (m, 3H), 1.21-1.30 (m, 2H), 1.18 (s, 6H). MS: m/z 363 (M+H). The second eluting compound corresponds to the desired isomer 2-(dimethylamino)-N-(trans)-4-(2-hydroxypropan-2-yl)cyclohexyl)thiazolo[4,5-b]pyridine-6-carboxamide (4.5 mg, 0.012 mmol, 27% yield). ¹H NMR (400 MHz, CD₃OD) δ ppm 8.74 (d, J=2 Hz, 1H), 8.48 (d, J=2 Hz, 1H), 3.84 (tt, J=12, 4 Hz, 1H), 3.30 (s, 6H), 2.03-2.14 (m, 2H), 1.92-2.02 (m, 2H), 1.32-1.47 (m, 3H), 1.22-1.32 (m, 2H), 1.19 (s, 6H). MS: m/z 363 (M+H).

### Example 10

### N-(trans-4-(2-Hydroxypropan-2-yl)cyclohexyl)-2-((1S,2R)-2-methylcyclopropyl)thieno[3,2-b]pyridine-6-carboxamide and N-(trans-4-(2-hydroxypropan-2-yl)cyclohexyl)-2-((1R,2S)-2-methylcyclopropyl)thieno[3,2-b]pyridine-6-carboxamide

[chemical structure]

To a mixture of 2-bromo-N-(trans)-4-(2-hydroxypropan-2-yl)cyclohexyl)thieno[3,2-b]pyridine-6-carboxamide (50 mg, 0.126 mmol, Example 1), (cis)-2-methylcyclopropyl)boronic acid (38 mg, 0.378 mmol), 2-dicyclohexylphosphino-2′,6′-dimethoxyphenyl (S-PHOS) (12 mg, 0.030 mmol) and Pd₂(dba)₃ (7 mg, 7.55 μmol) in toluene (3 mL) was added 2M Na₂CO₃ (0.189 mL, 0.378 mmol). The mixture was purged with N₂ for a few minutes, and then heated in a sealed tube at 110° C. for ˜15 h. Upon cooling, the reaction mixture was diluted with EtOAc and washed 1× with water and 1× with brine. The combined aqueous phases were back-extracted 1× with EtOAc. This EtOAc phase was washed 1× with brine. The organic phases were combined, dried over Na₂SO₄, filtered, and concentrated. The residue was purified by silica gel chromatography, eluting with 5-60% EtOAc:hexanes gradient. The compound was further purified by radial chromatography (1 mm chromatotron plate; 0-5% MeOH:CH₂Cl₂ gradient) to yield the product as a white solid. The product was dissolved in CH₂Cl₂ with a few drops of MeOH and added into stirring hexanes. The mixture was partially concentrated, and the solids were collected by filtration, washed with hexanes and dried under high vacuum at 50° C. to yield a racemic mixture of N-(trans-4-(2-hydroxypropan-2-yl)cyclohexyl)-2-((1S,2R)-2-methylcyclopropyl)thieno[3,2-b]pyridine-6-carboxamide and N-(trans-4-(2-hydroxypropan-2-yl)cyclohexyl)-2-((1R,2S)-2-methylcyclopropyl)thieno[3,2-b]pyridine-6-carboxamide (19 mg, 0.043 mmol, 35% yield, 85-90% purity by ¹H NMR). (Tested as racemic mixture in the assay disclosed herein)¹H NMR (400 MHz, CD₃SOCD₃) δ ppm 8.99 (d, J=2 Hz, 1H), 8.72 (d, J=2 Hz, 1H), 8.40 (d, J=8 Hz, 1H), 7.32 (s, 1H), 4.07 (s, 1H), 3.66-3.83 (m, 1H), 2.32-2.41 (m, 1H), 1.89-1.99 (m, 2H), 1.79-1.89 (m, 2H), 1.22-1.42 (m, 4H), 1.07-1.21 (m, 3H), 1.05 (s, 6H), 0.92 (d, J=6 Hz, 3H), 0.89 (m, 1H). MS: m/z 373 (M+H).

### Example 11

### 2-Bromo-N-(3-(2-hydroxypropan-2-yl)bicyclo[1.1.1]pentan-1-yl)thieno[3,2-b]pyridine-6-carboxamide

[chemical structure]

To a solution of 2-bromothieno[3,2-b]pyridine-6-carboxylic acid (50 mg, 0.194 mmol, Intermediate 1) and DIEA (0.044 mL, 0.252 mmol) in DMF (2 mL) was added HATU (88 mg, 0.232 mmol). After stirring for ˜2 min, 2-(3-aminobicyclo[1,1,1]pentan-1-yl)propan-2-ol hydrochloride (43 mg, 0.194 mmol, Intermediate 5) and additional DIEA (0.085 mL, 0.484 mmol) were added. After ˜1 h, water (˜7 mL) was slowly added to the reaction mixture. A light brown solid precipitated out. The mixture was stirred fora few minutes, and then the solids were collected by filtration, washed sequentially with water and with hexanes and dried under vacuum to give 2-bromo-N-(3-(2-hydroxypropan-2-yl)bicyclo[1.1.1]pentan-1-yl)thieno[3,2-b]pyridine-6-carboxamide (52 mg, 0.13 mmol, 67% yield) as a light brown solid. ¹H NMR (400 MHz, CD₃SOCD₃) δ ppm 9.19 (s, 1H), 9.02 (d, J=2 Hz, 1H), 8.82 (d, J=2 Hz, 1H), 7.87 (s, 1H), 4.21 (s, 1H), 1.93 (s, 6H), 1.09 (s, 6H). MS: m/z 381/383 (M+H) for Br isotopes.

### Example 12

### 2-Cyclopropyl-N-(3-(2-hydroxypropan-2-yl)bicyclo[1.1.1]pentan-1-yl)thieno[3,2-b]pyridine-6-carboxamide

[chemical structure]

To a mixture of 2-bromo-N-(3-(2-hydroxypropan-2-yl)bicyclo[1,1,1]pentan-1-yl)thieno[3,2-b]pyridine-6-carboxamide (45 mg, 0.118 mmol, Example 11), cyclopropylboronic acid (30 mg, 0.354 mmol), 2-dicyclohexylphosphino-2′,6′-dimethoxyphenyl (S-PHOS) (12 mg, 0.028 mmol) and Pd₂(dba)₃ (7 mg, 7.08 μmol) in toluene (3 mL) was added 2M Na₂CO₃ (0.177 mL, 0.354 mmol). The mixture was purged with N₂ for a few minutes, and then it was heated in a sealed tube at 110° C. for ˜15 h. Upon cooling, the reaction mixture was diluted with EtOAc and washed 1× with water and 1× with brine. The combined aqueous phases were back-extracted 1× with EtOAc. This EtOAc phase was washed 1× with brine. The organic phases were combined, dried over Na₂SO₄, filtered, and concentrated. The residue was purified by reverse-phase chromatography, eluting with 0-100% MeCN:water with 0.1% TFA. The fractions with product were partially concentrated under vacuum, down to the aqueous phase. The residual solution was basified with aq. satd. K₂CO₃ solution and extracted with EtOAc. The organic phase was washed 1× with brine. The combined aqueous phases were back-extracted 1× with EtOAc. This EtOAc phase was washed 1× with brine. The EtOAc phases were combined, dried over Na₂SO₄, filtered, and concentrated. The residue was dissolved in CH₂Cl₂ (2 mL) and slowly diluted with hexanes (˜5 mL). The mixture was partially concentrated under a N₂ stream. The white solids were collected by filtration, washed with hexanes and dried by air suction to give 2-cyclopropyl-N-(3-(2-hydroxypropan-2-yl)bicyclo[1,1,1]pentan-1-yl)thieno[3,2-b]pyridine-6-carboxamide (21 mg, 0.058 mmol, 49% yield). ¹H NMR (400 MHz, CD3SOCD₃) δ ppm 9.08 (s, 1H), 8.96 (d, J=2 Hz, 1H), 8.69 (dd, J=2, 1 Hz, 1H), 7.32 (d, J=1 Hz, 1H), 4.20 (s, 1H), 2.31-2.41 (m, 1H), 1.92 (s, 6H), 1.15-1.22 (m, 2H), 1.08 (s, 6H), 0.88-0.95 (m, 2H). MS: m/z 343 (M+H).

### Example 13

### 2-Cyclopropyl-N-(3-(2-hydroxypropan-2-yl)bicyclo[1.1.1]pentan-1-yl)thiazolo[4,5-b]pyridine-6-carboxamide

[chemical structure]

To a solution of 2-cyclopropylthiazolo[4,5-b]pyridine-6-carboxylic acid (40 mg, 0.182 mmol, Intermediate 4) in DMF (1 mL) was added DIEA (31 mg, 0.236 mmol), followed by HATU (83 mg, 0.218 mmol). After ˜5 min, 2-(3-aminobicyclo[1,1,1]pentan-1-yl)propan-2-ol hydrochloride (40 mg, 0.182 mmol, Intermediate 5) was added, followed by additional DIEA (79 μl, 0.454 mmol). After ˜1 h, water (˜5 mL) was added slowly, dropwise. The reaction mixture was stirred overnight, and then it was partitioned between EtOAc and water. The organic phase was washed 1× with satd. K₂CO₃ solution and 1× with brine. The combined aqueous phases were back-extracted 2× with EtOAc. These EtOAc phases were washed 1× with brine. The organic phases were combined, dried over Na₂SO₄, filtered, and concentrated. The residue was purified by silica gel chromatography, eluting with 5-50% (3:1 EtOAc:EtOH):hexanes gradient to yield the product as a white solid. The solid was dissolved in CH₂Cl₂ with a few drops of MeOH, and this solution was added into stirring hexanes. The mixture was then partially concentrated under a N₂ stream. The solids were collected by filtration, washed with hexanes and dried at 50° C. under high vacuum overnight to give 2-cyclopropyl-N-(3-(2-hydroxypropan-2-yl)bicyclo[1.1.1]pentan-1-yl)thiazolo[4,5-b]pyridine-6-carboxamide (53 mg, 0.147 mmol, 81% yield) as a white solid. ¹H NMR (400 MHz, CD3SOCD₃) δ ppm 9.16 (s, 1H), 8.99 (d, J=2 Hz, 1H), 8.87 (d, J=2 Hz, 1H), 4.21 (s, 1H), 2.64 (tt, J=8, 5 Hz, 1H), 1.93 (s, 6H), 1.29-1.41 (m, 2H), 1.19-1.29 (m, 2H), 1.09 (s, 6H). MS: m/z 344 (M+H).

### Example 14

### 2-Cyclobutyl-N-((trans)-4-(2-hydroxypropan-2-yl)cyclohexyl)thiazolo[4,5-b]pyridine-6-carboxamide

[chemical structure]

To a solution of 2-cyclobutylthiazolo[4,5-b]pyridine-6-carboxylic acid (30 mg, 0.128 mmol, Intermediate 6) in DMF (1 mL) was added DIEA (0.029 mL, 0.166 mmol), followed by HATU (58 mg, 0.154 mmol). After ˜5 min, 2-((trans)-4-aminocyclohexyl)propan-2-ol (23 mg, 0.147 mmol) was added, followed by additional DIEA (0.029 mL, 0.166 mmol). After ˜45 min, water (˜8 mL) was added slowly, dropwise. After a few minutes of stirring, precipitation occurred. The solids were collected by filtration, washed sequentially with water and with hexanes, and dried under high vacuum at 50° C. overnight to give 2-cyclobutyl-N-((trans)-4-(2-hydroxypropan-2-yl)cyclohexyl)thiazolo[4,5-b]pyridine-6-carboxamide (41 mg, 0.104 mmol, 81% yield) as a white solid. ¹H NMR (400 MHz, CD3SOCD₃) δ ppm 9.06 (d, J=2 Hz, 1H), 8.95 (d, J=2 Hz, 1H), 8.51 (d, J=8 Hz, 1H), 4.07 (s, 1H), 4.04-4.14 (m, 1H), 3.64-3.83 (m, 1H), 2.37-2.49 (m, 4H), 2.04-2.18 (m, 1H), 1.90-2.03 (m, 3H), 1.85 (m, 2H), 1.25-1.38 (m, 2H), 1.07-1.25 (m, 3H), 1.06 (s, 6H). MS: m/z 374 (M+H).

### Example 15

### (Racemic)-2-cyclopropyl-N-(6-(2-hydroxypropan-2-yl)spiro[3.3]heptan-2-yl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 min, 2-(6-aminospiro[3.3]heptan-2-yl)propan-2-ol (32 mg, 0.150 mmol) was added, followed by additional DIEA (0.031 mL, 0.177 mmol). After ˜45 min, water (˜7 mL) was added slowly, dropwise to the reaction mixture. A yellow precipitate formed. The mixture was stirred for a few minutes, and then the solids were collected by filtration, washed sequentially with water and with hexanes and dried at 60° C. under high vacuum to give (racemic)-2-cyclopropyl-N-(6-(2-hydroxypropan-2-yl)spiro[3.3]heptan-2-yl)thiazolo[4,5-b]pyridine-6-carboxamide (32 mg, 0.082 mmol, 60% yield) as a yellow solid. ¹H NMR (400 MHz, CD₃SOCD₃) δ ppm 9.00 (d, J=2 Hz, 1H), 8.88 (d, J=2 Hz, 1H), 8.80 (d, J=7 Hz, 1H), 4.31 (m, 1H), 4.02 (s, 1H), 2.58-2.69 (m, 1H), 2.36-2.46 (m, 1H), 2.04-2.23 (m, 3H), 1.85-2.03 (m, 4H), 1.66-1.78 (m, 1H), 1.29-1.39 (m, 2H), 1.20-1.29 (m, 2H), 0.96 (d, J=5 Hz, 6H). MS: m/z 372 (M+H).

### Example 16

### 2-Cyclopropyl-N-((trans)-4-hydroxycyclohexyl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (31 μl, 0.177 mmol), followed by HATU (62 mg, 0.163 mmol). After ˜5 min, (trans)-4-aminocyclohexan-1-ol hydrochloride (26 mg, 0.170 mmol) was added, followed by additional DIEA (60 μl, 0.341 mmol). After ˜45 min, water (˜8 mL) was slowly added to the reaction mixture. After stirring at rt overnight, the mixture was partitioned between EtOAc and satd. K₂CO₃ solution. The aqueous phase was extracted 2× with EtOAc. The combined organic phases were washed 1× with a very small amount of satd. brine, dried over Na₂SO₄, filtered, and concentrated. The residue was purified by silica gel chromatography, eluting with 5-70% (3:1 EtOAc:EtOH):hexanes gradient. The product was then crystallized by dissolving it in CH₂Cl₂ with a few drops of MeOH and adding it into stirring hexanes. The mixture was then partially concentrated under a N₂ stream to give 2-cyclopropyl-N-((trans)-4-hydroxycyclohexyl)thiazolo[4,5-b]pyridine-6-carboxamide (34 mg, 0.102 mmol, 75% yield) as a white solid. ¹H NMR (400 MHz, CD₃SOCD₃) δ ppm 8.99 (d, J=2 Hz, 1H), 8.88 (d, J=2 Hz, 1H), 8.45 (d, J=8 Hz, 1H), 4.59 (d, J=4 Hz, 1H), 3.68-3.83 (m, 1H), 3.36-3.48 (m, 1H), 2.59-2.70 (m, 1H), 1.79-1.94 (m, 4H), 1.19-1.45 (m, 8H). MS: m/z 318 (M+H).

### Example 17

### 2-Cyclopropyl-N-((trans)-4-hydroxy-4-methylcyclohexyl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 minutes, (trans)-4-amino-1-methylcyclohexan-1-ol (21 mg, 0.163 mmol) was added, followed by additional DIEA (0.031 mL, 0.177 mmol). After ˜45 min, water (˜8 mL) was slowly added to the reaction mixture. After stirring at rt overnight, the mixture was partitioned between EtOAc and satd. K₂CO₃ solution. To the aqueous phase was added some solid NaCl and then it was extracted 2× with EtOAc. The combined organic phases were washed 1× with a very small amount of satd. brine, dried over Na₂SO₄, filtered, and concentrated. The residue was purified by silica gel chromatography, eluting with 5-70% (3:1 EtOAc:EtOH):hexanes gradient. The product was subsequently crystallized from CH₂Cl₂:hexanes to give 2-cyclopropyl-N-((trans)-4-hydroxy-4-methylcyclohexyl)thiazolo[4,5-b]pyridine-6-carboxamide (38 mg, 0.109 mmol, 80% yield) as a white solid. ¹H NMR (400 MHz, CD₃SOCD₃) δ ppm 8.99 (d, J=2 Hz, 1H), 8.88 (d, J=2 Hz, 1H), 8.42 (d, J=8 Hz, 1H), 4.31 (s, 1H), 3.77-3.94 (m, 1H), 2.64 (tt, J=8.5 Hz, 1H), 1.74-1.85 (m, 2H), 1.57-1.67 (m, 2H), 1.39-1.55 (m, 4H), 1.30-1.38 (m, 2H), 1.21-1.29 (m, 2H), 1.16 (s, 3H). MS: m/z 332 (M+H).

### Example 18

### 2-Cyclopropyl-N-((trans)-4-(2-hydroxyethoxy)cyclohexyl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 min, 2-(((trans)-4-aminocyclohexyl)oxy)ethan-1-ol (26 mg, 0.163 mmol) was added, followed by additional DIEA (0.031 mL, 0.177 mmol). After ˜45 min, water (˜8 mL) was slowly added to the reaction mixture. After stirring at rt overnight, the mixture was partitioned between EtOAc and satd. K₂CO₃ solution. To the aqueous phase was added some solid NaCl and then extracted 2× with EtOAc. The combined organic phases were washed 1× with a very small amount of satd. brine, dried over Na₂SO₄, filtered, and concentrated. The residue was purified by silica gel chromatography, eluting with 5-60% (3:1 EtOAc:EtOH):hexanes gradient. The product was then crystallized by dissolving it in CH₂Cl₂ (3 mL) with a few drops of MeOH and adding it into stirring hexanes (˜4 mL). The mixture was then partially concentrated under a N₂ stream. The solids were collected by filtration, washed with hexanes and dried under high vacuum to give 2-cyclopropyl-N-((trans)-4-(2-hydroxyethoxy)cyclohexyl)thiazolo[4,5-b]pyridine-6-carboxamide (42 mg, 0.11 mmol, 81% yield) as a white solid. ¹H NMR (400 MHz, CD3SOCD₃) δ ppm 9.00 (d, J=2 Hz, 1H), 8.88 (d, J=2 Hz, 1H), 8.48 (d, J=8 Hz, 1H), 4.57 (t, J=5 Hz, 1H), 3.72-3.86 (m, 1H), 3.40-3.53 (m, 4H), 3.20-3.30 (m, 1H), 2.59-2.70 (m, 1H), 1.97-2.11 (m, 2H), 1.83-1.95 (m, 2H), 1.18-1.46 (m, 8H). MS: m/z 362 (M+H).

### Example 19

### (S)-2-Cyclopropyl-N-(2-oxopyrrolidin-3-yl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 min, (S)-3-aminopyrrolidin-2-one (16 mg, 0.163 mmol) was added, followed by additional DIEA (0.031 mL, 0.177 mmol). After ˜45 min, water (˜8 mL) was slowly added to the reaction mixture. After stirring at rt overnight, the mixture was partitioned between EtOAc and satd. K₂CO₃ solution. To the aqueous phase was added some solid NaCl and then it was extracted 2× with EtOAc. The combined organic phases were washed 1× with a very small amount of satd. brine, dried over Na₂SO₄, filtered, and concentrated. The residue was purified by silica gel chromatography, eluting with 10-100% (3:1 EtOAc:EtOH):hexanes gradient. The product was then crystallized by taking it in CH₂Cl₂ with a few drops of MeOH, and adding the suspension into stirring hexanes. The mixture was partially concentrated under a N₂ stream. The white solids were collected by filtration, washed with hexanes, and dried under high vacuum at 60° C. to give (S)-2-cyclopropyl-N-(2-oxopyrrolidin-3-yl)thiazolo[4,5-b]pyridine-6-carboxamide (28 mg, 0.092 mmol, 65% yield) as an off-white solid. ¹H NMR (400 MHz, CD3SOCD₃) δ ppm 9.04 (d, J=2 Hz, 1H), 8.96 (d, J=8 Hz, 1H), 8.93 (d, J=2 Hz, 1H), 7.92 (s, 1H), 4.61 (dt, J=10, 8 Hz, 1H), 3.21-3.29 (m, 2H), 2.59-2.71 (m, 1H), 2.31-2.46 (m, 1H), 1.95-2.11 (m, 1H), 1.30-1.38 (m, 2H), 1.23-1.30 (m, 2H). MS: m/z 303 (M+H).

### Example 20

### 2-Cyclopropyl-N-((trans)-4-(hydroxymethyl)cyclohexyl)thiazolo[4,5-b]pyridine-6-carboxamide

[chemical structure]

To a solution of 2-cyclopropylthiazolo[4,5-b]pyridine-6-carboxylic acid (100 mg, 0.454 mmol, Intermediate 4) in DMF (1 mL) was added DIEA (0.103 mL, 0.590 mmol), followed by HATU (207 mg, 0.545 mmol). After ˜5 min, ((trans)-4-aminocyclohexyl)methanol hydrochloride (90 mg, 0.545 mmol) was added, followed by additional DIEA (0.198 mL, 1.135 mmol). After ˜1 h, water was added slowly, dropwise. A beige solid precipitated out. The mixture was stirred for a few minutes and then the solids were collected by filtration, washed sequentially with water and with hexanes, and dried under high vacuum at 60° C. The crude product was purified by silica gel chromatography, eluting with 10-100% (3:1 EtOAc:EtOH):hexanes to give 2-cyclopropyl-N-((trans)-4-(hydroxymethyl)cyclohexyl)thiazolo[4,5-b]pyridine-6-carboxamide (44 mg, 0.126 mmol, 28% yield) as a white solid. ¹H NMR (400 MHz, CD₃SOCD₃) δ ppm 9.01 (d, J=2 Hz, 1H), 8.88 (d, J=2 Hz, 1H), 8.49 (d, J=8 Hz, 1H), 4.43 (t, J=5 Hz, 1H), 3.68-3.84 (m, 1H), 3.24 (t, J=6 Hz, 2H), 2.58-2.70 (m, 1H), 1.86-1.97 (m, 2H), 1.75-1.84 (m, 2H), 1.22-1.42 (m, 7H), 0.92-1.06 (m, 2H). MS: m/z 332 (M+H).

### Example 21

### 2-Cyclopropyl-N-((trans)-4-(3,3-difluoroazetidin-1-yl)cyclohexyl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 min, (trans)-4-(3,3-difluoroazetidin-1-yl)cyclohexan-1-amine (31 mg, 0.163 mmol) was added, followed by additional DIEA (0.031 mL, 0.177 mmol). After a few minutes, a beige solid precipitated out. After ˜1 h, while stirring, water (˜3 mL) was added to the heterogeneous mixture. After ˜30 min, the solids were collected by filtration, washed sequentially with water and with hexanes, and dried under high vacuum at 60° C. overnight to give 2-cyclopropyl-N-((trans)-4-(3,3-difluoroazetidin-1-yl)cyclohexyl)thiazolo[4,5-b]pyridine-6-carboxamide (46 mg, 0.111 mmol, 82% yield) as a light beige solid. ¹H NMR (400 MHz, CD3SOCD₃) δ ppm 9.00 (d, J=2 Hz, 1H), 8.88 (d, J=2 Hz, 1H), 8.49 (d, J=8 Hz, 1H), 3.70-3.83 (m, 1H), 3.55 (t, J=12 Hz, 4H), 2.64 (tt, J=8, 5 Hz, 1H), 2.07-2.21 (m, 1H), 1.84-1.95 (m, 2H), 1.71-1.83 (m, 2H), 1.29-1.46 (m, 4H), 1.19-1.29 (m, 2H), 0.99-1.16 (m, 2H). MS: m/z 393 (M+H).

### Example 22

### 2-Cyclopropyl-N-((trans)-3-(2-hydroxypropan-2-yl)cyclobutyl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 min, 2-((trans)-3-aminocyclobutyl)propan-2-ol hydrochloride (23 mg, 0.136 mmol) was added, followed by additional DIEA (0.059 mL, 0.341 mmol). The reaction mixture was stirred at rt for ˜1 h. The mixture was purified directly (no work-up) by reverse-phase chromatography, eluting with ACN:water with 0.1% NH₄OH (20-60%). The fractions with product were lyophilized to give 2-cyclopropyl-N-((trans)-3-(2-hydroxypropan-2-yl)cyclobutyl)thiazolo[4,5-b]pyridine-6-carboxamide (34 mg, 0.097 mmol, 72% yield) as an off-white solid. ¹H NMR (400 MHz, CD3SOCD₃) δ ppm 9.03 (d, J=2 Hz, 1H), 8.90 (d, J=2 Hz, 1H), 8.86 (d, J=7 Hz, 1H), 4.29-4.41 (m, 1H), 4.26 (s, 1H), 2.64 (tt, J=8, 5 Hz, 1H), 2.20-2.37 (m, 3H), 1.99-2.11 (m, 2H), 1.30-1.38 (m, 2H), 1.22-1.29 (m, 2H), 1.06 (s, 6H). MS: m/z 332 (M+H).

### Example 23

### 2-Cyclopropyl-N-(trans-4-((1,1-difluoropropan-2-yl)amino)cyclohexyl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 min, (trans)-N1-(1,1-difluoropropan-2-yl)cyclohexane-1,4-diamine (31 mg, 0.163 mmol) was added, followed by additional DIEA (0.031 mL, 0.177 mmol). The reaction mixture was stirred at rt for ˜1 h. The mixture was purified directly (no work-up) by reverse-phase chromatography, eluting with ACN:water with 0.1% NH₄OH (20-60%). The fractions with product were lyophilized to give 2-cyclopropyl-N-(trans-4-((1,1-difluoropropan-2-yl)amino)cyclohexyl)thiazolo[4,5-b]pyridine-6-carboxamide (36 mg, 0.087 mmol, 64% yield) as an off-white solid. ¹H NMR (400 MHz, CD3SOCD₃) δ ppm 9.00 (d, J=2 Hz, 1H), 8.88 (d, J=2 Hz, 1H), 8.48 (br d, J=7 Hz, 1H), 5.81 (t, 0=57 Hz, 1H), 3.68-3.83 (m, 1H), 2.91-3.10 (m, 1H), 2.59-2.72 (m, 1H), 1.80-2.04 (m, 4H), 1.45-1.60 (m, 1H), 1.29-1.45 (m, 5H), 1.21-1.29 (m, 2H), 0.98-1.20 (m, 5H). MS: m/z 395 (M+H).

### Example 24

### 2-Cyclopropyl-N-((3R,6S)-6-(2-hydroxypropan-2-yl)tetrahydro-2H-pyran-3-yl)thiazolo[4,5-b]pyridine-6-carboxamide and 2-Cyclopropyl-N-((3S,6R)-6-(2-hydroxypropan-2-yl)tetrahydro-2H-pyran-3-yl)thiazolo[4,5-b]pyridine-6-carboxamide

[chemical structure]

To a solution of 2-cyclopropylthiazolo[4,5-b]pyridine-6-carboxylic acid (30 mg, 0.136 mmol, Intermediate 4) in DMF (1 mL) was added DIEA (0.031 mL, 0.177 mmol), followed by HATU (62 mg, 0.163 mmol). After ˜5 min, racemic 2-((trans)-5-aminotetrahydro-2H-pyran-2-yl)propan-2-ol (26 mg, 0.163 mmol) was added, followed by additional DIEA (0.031 mL, 0.177 mmol). The reaction mixture was stirred at rt for ˜1 h. The mixture was purified directly (no work-up) by reverse-phase chromatography, eluting with ACN:water with 0.1% NH₄OH (20-60%). The fractions with product were lyophilized and the resulting solids were dried at 70° C. under high vacuum overnight to give a racemic mixture of 2-cyclopropyl-N-((3R,6S)-6-(2-hydroxypropan-2-yl)tetrahydro-2H-pyran-3-yl)thiazolo[4,5-b]pyridine-6-carboxamide and 2-cyclopropyl-N-((3S,6R)-6-(2-hydroxypropan-2-yl)tetrahydro-2H-pyran-3-yl)thiazolo[4,5-b]pyridine-6-carboxamide (33 mg, 0.087 mmol, 64% yield) as a white solid. (Tested as racemic mixture in the assay disclosed herein)¹H NMR (400 MHz, CD₃SOCD₃) δ ppm 9.01 (d, J=2 Hz, 1H), 8.89 (d, J=2 Hz, 1H), 8.48 (d, J=7 Hz, 1H), 4.28 (s, 1H), 3.94-4.01 (m, 1H), 3.82-3.93 (m, 1H), 3.14 (t, J=10 Hz, 1H), 2.99 (dd, J=11.2 Hz, 1H), 2.64 (tt, J=8, 5 Hz, 1H), 1.97-2.07 (m, 1H), 1.77-1.87 (m, 1H), 1.51-1.66 (m, 1H), 1.36-1.46 (m, 1H), 1.30-1.36 (m, 2H), 1.22-1.29 (m, 2H), 1.11 (s, 3H), 1.05 (s, 3H). MS: m/z 362 (M+H).

### Example 25

### 2-(2-Fluoropropan-2-yl)-N-(trans-4-(2-hydroxypropan-2-yl)cyclohexyl)thiazolo[4,5-b]pyridine-6-carboxamide

[chemical structure]

To a stirred solution of methyl 2-(2-fluoropropan-2-yl)thiazolo[4,5-b]pyridine-6-carboxylate (0.10 g, 0.393 mmol, Intermediate 7) in a mixture of methanol (1 mL) and tetrahydrofuran (THF) (1 mL) was added a 4M aqueous solution of NaOH (0.157 g, 3.93 mmol). The reaction mixture was stirred for 2 hours at rt, and then it was acidified with 6N HCl and concentrated to dryness under vacuum. The residue was mixed with toluene and concentrated twice to remove traces of water. The residue was then dissolved in DMF (2 mL) and treated with HATU (0.224 g, 0.590 mmol), followed by DIEA (0.687 mL, 3.93 mmol). After stirring for 15 minutes at rt, 2-(trans-4-aminocyclohexyl)propan-2-ol (0.093 g, 0.590 mmol) was added, and stirring was continued overnight. The reaction mixture was poured into water (20 mL) and extracted with EtOAc (3×25 mL). The combined EtOAc layers were washed with water and brine, dried over Na₂SO₄ and concentrated under vacuum. The residue was purified by silica gel chromatography, eluting with 0-60% (3:1 EtOAc:EtOH):hexanes gradient to give 2-(2-fluoropropan-2-yl)-N-(trans-4-(2-hydroxypropan-2-yl)cyclohexyl)thiazolo[4,5-b]pyridine-6-carboxamide (0.10 g, 0.25 mmol, 64% yield). ¹H NMR (400 MHz, CD₃SOCD₃) δ: 9.13 (d, J=2 Hz, 1H), 9.06 (d, J=2 Hz, 1H), 8.57 (d, J=8 Hz, 1H), 4.08 (s, 1H), 3.67-3.81 (m, 1H), 1.79-2.01 (m, 4H), 1.88 (d, J=22 Hz, 6H), 1.25-1.40 (m, 2H), 0.98-1.25 (m, 3H), 1.05 (s, 6H). MS: m/z 380 (M+H).

### Example 26

### 2-Cyclopropyl-N-(trans-4-(cyclopropyl(hydroxy)methyl)cyclohexyl)thiazolo[4,5-b]pyridine-6-carboxamide

[chemical structure]

To a suspension of 2-cyclopropyl-N-((trans)-4-formylcyclohexyl)thiazolo[4,5-b]pyridine-6-carboxamide (35 mg, 0.070 mmol, Intermediate 8) in THF (4 mL), cooled at −78° C., was added cyclopropane magnesium bromide (1 M in 2-methyl-THF) (0.175 mL, 0.175 mmol) dropwise. The mixture was stirred at −78° C., and after ˜3 h, additional cyclopropane magnesium bromide (1M in 2-methyl-THF) (0.125 mL, 0.125 mmol) was added. After another 20 min, while in the cold bath, satd. NH₄Cl (˜5 mL) solution was added. Upon warming, the mixture was partitioned between EtOAc and water. The organic phase was washed 1× with brine, dried over Na₂SO₄, filtered, and concentrated. The residue was purified by reverse-phase chromatography, eluting with 0-100% MeCN:water with 0.1% NH₄OH. The fraction with product was concentrated in a rotary evaporator, and then it was repurified by reverse-phase chromatography, eluting with 0-100% MeCN-water with 0.1% TFA. The fraction with product was basified with satd. K₂CO₃ solution and partially concentrated under vacuo, down to the aqueous phase. A solid precipitated out. The whole mixture was partitioned between EtOAc and water. The organic phase was washed with brine, dried over Na₂SO₄, filtered, and concentrated to give 2-cyclopropyl-N-(trans-4-(cyclopropyl(hydroxy)methyl)cyclohexyl)thiazolo[4,5-b]pyridine-6-carboxamide (5.5 mg, 0.014 mmol, 20% yield) as a white solid. ¹H NMR (400 MHz, CD3SOCD₃) δ ppm 9.01 (d, J=2 Hz, 1H), 8.88 (d, J=2 Hz, 1H), 8.48 (d, J=8 Hz, 1H), 4.34 (br d, J=1 Hz, 1H), 3.64-3.84 (m, 1H), 2.55-2.71 (m, 2H), 1.88-2.03 (m, 3H), 1.80-1.88 (m, 1H), 1.09-1.42 (m, 9H), 0.75-0.87 (m, 1H), 0.29-0.47 (m, 2H), 0.12-0.27 (m, 2H). MS: m/z 372 (M+H).

### Example 27

### 2-(tert-Butyl)-N-(trans-4-(2-hydroxypropan-2-yl)cyclohexyl)thiazolo[4,5-b]pyridine-6-carboxamide

[chemical structure]

To a stirred solution of methyl 2-(tert-butyl)thiazolo[4,5-b]pyridine-6-carboxylate (0.110 g, 0.439 mmol, Intermediate 9) in a mixture of MeOH (1 mL) and THF (1 mL) was added a 4M aqueous solution of NaOH (0.176 g, 4.39 mmol) at rt. The reaction mixture was stirred for 2 h at rt, and then it was acidified with 6N HCl and concentrated to dryness under vacuum. The residue was mixed with toluene and concentrated to remove traces of water. The residue was then dissolved in DMF (2 mL) and to this was added HATU (0.251 g, 0.659 mmol), followed by DIEA (0.768 mL, 4.39 mmol). After stirring at rt for 15 min, 2-(trans-4-aminocyclohexyl)propan-2-ol (0.104 g, 0.659 mmol) was added, and the mixture was stirred overnight. The reaction mixture was poured into water (20 mL) and extracted with EtOAc (3×25 mL). The combined EtOAc layers were washed with water and brine, dried over Na₂SO₄ and concentrated under vacuum. The residue was purified by silica gel chromatography, eluting with 0-60% (3:1 EtOAc:EtOH):hexanes gradient to give 2-(tert-butyl)-N-(trans-4-(2-hydroxypropan-2-yl)cyclohexyl)thiazolo[4,5-b]pyridine-6-carboxamide (0.120 g, 0.304 mmol, 69% yield). ¹H NMR (400 MHz, CD3SOCD₃) δ: 9.08 (d, J=2 Hz, 1H), 8.97 (d, J=2 Hz, 1H), 8.51 (d, J=8 Hz, 1H), 4.07 (br s, 1H), 3.66-3.82 (m, 1H), 1.94 (d, J=10 Hz, 2H), 1.85 (d, J=11 Hz, 2H), 1.50 (s, 9H), 1.25-1.40 (m, 2H), 0.99-1.25 (m, 9H). MS: m/z 376 (M+H).

### Example 28

### Racemic 2-cyclopropyl-N-(trans-4-(1-hydroxy-2-(methylsulfonyl)ethyl)cyclohexyl)thiazolo[4,5-b]pyridine-6-carboxamide

[chemical structure]

To a solution of 2-cyclopropylthiazolo[4,5-b]pyridine-6-carboxylic acid (25 mg, 0.114 mmol, Intermediate 4) in DMF (1 mL) was added DIEA (0.030 mL, 0.172 mmol), followed by HATU (52 mg, 0.136 mmol). After ˜5 min, racemic 1-((trans)-4-aminocyclohexyl)-2-(methylsulfonyl)ethan-1-ol trifluoroacetate salt (38 mg, 0.114 mmol, Intermediate 10) was added, followed by additional DIEA (0.090 mL, 0.515 mmol). After ˜45 min, water (˜5 mL) was added dropwise. After stirring for ˜30 min, the precipitated solids were collected by filtration, washed sequentially with water and with hexanes, and dried under vacuum to give racemic 2-cyclopropyl-N-(trans-4-(1-hydroxy-2-(methylsulfonyl)ethyl)cyclohexyl)thiazolo[4,5-b]pyridine-6-carboxamide (36 mg, 0.081 mmol, 71% yield) as a light beige solid. ¹H NMR (400 MHz, CD₃SOCD₃) δ ppm 9.00 (d, J=2 Hz, 1H), 8.88 (d, J=2 Hz, 1H), 8.49 (d, J=8 Hz, 1H), 5.19 (d, J=6 Hz, 1H), 3.78-3.86 (m, 1H), 3.68-3.78 (m, 1H), 3.26 (dd, J=15, 10 Hz, 1H), 3.08 (br d, J=14 Hz, 1H), 3.01 (s, 3H), 2.60-2.70 (m, 1H), 1.88-1.98 (m, 2H), 1.65-1.82 (m, 2H), 1.11-1.44 (m, 9H). MS: m/z 424 (M+H).

### Example 29

### 2-(Azetidin-1-yl)-N-(trans-4-(2-hydroxypropan-2-yl)cyclohexyl)thiazolo[4,5-b]pyridine-6-carboxamide

[chemical structure]

To a solution of 2-(azetidin-1-yl)thiazolo[4,5-b]pyridine-6-carboxylic acid (35 mg, 0.15 mmol, Intermediate 11) in DMF (3 mL) was added DIEA (0.131 mL, 0.750 mmol), followed by HATU (68 mg, 0.180 mmol). After ˜5 min, 2-((trans)-4-aminocyclohexyl)propan-2-ol (47 mg, 0.30 mmol) was added, followed by additional DIEA (0.079 mL, 0.45 mmol). After ˜30 min, the reaction mixture was partitioned between satd. K₂CO₃ solution and EtOAc. The EtOAc phase was washed 1× with satd. K₂CO₃ solution and 1× with brine. The combined aqueous phases were back-extracted 2× with EtOAc. These organics were combined and washed 1× with brine. All the EtOAc phases were combined, dried over Na₂SO₄, filtered, and concentrated. The residue was purified by silica gel chromatography, eluting with 20-100% (3:1 EtOAc:EtOH):hexanes gradient. The isolated product was dissolved in CH₂Cl₂ (3 mL) with a few drops of MeOH and added into stirring hexanes (˜3 mL). A white solid precipitated out. The mixture was partially concentrated under a N₂ stream. The solids were collected by filtration, washed with hexanes, and dried under high vacuum to give 2-(azetidin-1-yl)-N-(trans-4-(2-hydroxypropan-2-yl)cyclohexyl)thiazolo[4,5-b]pyridine-6-carboxamide (42 mg, 0.107 mmol, 71% yield) as a white solid. ¹H NMR (400 MHz, CD3SOCD₃) δ ppm 8.75 (d, J=2 Hz, 1H), 8.56 (d, J=2 Hz, 1H), 8.25 (d, J=8 Hz, 1H), 4.22 (t, J=8 Hz, 4H), 4.06 (br s, 1H), 3.63-3.77 (m, 1H), 2.45-2.50 (m, 2H), 1.78-1.96 (m, 4H), 1.23-1.37 (m, 2H), 1.06-1.23 (m, 3H), 1.05 (s, 6H). MS: m/z 375 (M+H).

### Example 30

### N-(trans-4-(2-Hydroxypropan-2-yl)cyclohexyl)-2-isopropylthiazolo[4,5-b]pyridine-6-carboxamide

[chemical structure]

To a stirred solution of methyl 2-isopropylthiazolo[4,5-b]pyridine-6-carboxylate (0.070 g, 0.296 mmol, Intermediate 12) in MeOH (1 mL) and THF (1 mL) was added a 4M aqueous solution of NaOH (0.118 g, 2.96 mmol). The reaction mixture was stirred for 2 h at rt, and then it was acidified with 6N HCl and concentrated to dryness under vacuum. The residue was mixed with toluene and concentrated twice to remove traces of water. The residue was dissolved in DMF (2 mL) and treated with HATU (0.169 g, 0.444 mmol), followed by DIEA (0.517 mL, 2.96 mmol). After stirring for 15 min, 2-(trans-A-aminocyclohexyl)propan-2-ol (0.070 g, 0.444 mmol) was added and the mixture was stirred at rt overnight. The reaction mixture was poured into water (20 mL) and extracted with EtOAc (3×25 mL). The combined EtOAc layers were washed with water and brine, dried over Na₂SO₄ and concentrated under vacuum. The residue was purified by silica gel chromatography, eluting with 0-80% (3:1 EtOAc:EtOH):hexanes gradient to give N-(trans-4-(2-hydroxypropan-2-yl)cyclohexyl)-2-isopropylthiazolo[4,5-b]pyridine-6-carboxamide (0.045 g, 0.118 mmol, 40% yield). ¹H NMR (400 MHz, CD₃SOCD₃) δ: 9.06 (d, J=2 Hz, 1H), 8.96 (d, J=2 Hz, 1H), 8.51 (d, J=8 Hz, 1H), 3.66-3.84 (m, 1H), 4.1 (br s, 1H), 3.50 (sep, J=7 Hz, 1H), 1.80-1.99 (m, 4H), 1.44 (d, J=7 Hz, 6H), 1.26-1.38 (m, 2H), 1.07-1.24 (m, 3H), 1.05 (s, 6H). MS: m/z 362 (M+H).

### Example 31

### 2-Cyclopropyl-N-(1-(1-methyl-1H-tetrazol-5-yl)piperidin-4-yl)thiazolo[4,5-b]pyridine-6-carboxamide

[chemical structure]

N,N-Diisopropylethylamine (0.202 mL, 1.155 mmol) was added to a solution of 2-cyclopropylthiazolo[4,5-b]pyridine-6-carboxylic acid (0.064 g, 0.289 mmol, Intermediate 4) in dichloromethane (1.44 mL) at rt. Then, 1-(1-methyl-1H-tetrazol-5-yl)piperidin-4-amine hydrochloride (0.095 g, 0.433 mmol, Enamine Building Blocks) was added and the reaction mixture was stirred for 5 minutes. Then, n-propylphosphonic acid anhydride (0.344 mL, 0.578 mmol, 50 wt % in EtOAc) was added and the reaction mixture was stirred for 16 hours. The reaction mixture was concentrated. The resulting residue was purified by reverse-phase HPLC, eluting with acetonitrile:water with 0.1% ammonium hydroxide (5:95 to 100:0), then further purified by silica gel chromatography, eluting with methanol:ethyl acetate (0:1 to 2:3) to give 2-cyclopropyl-N-(1-(1-methyl-1H-tetrazol-5-yl)piperidin-4-yl)thiazolo[4,5-b]pyridine-6-carboxamide (0.048 g, 0.119 mmol, 41% yield). ¹H NMR (400 MHz, CD₃SOCD₃) δ 9.01 (d, J=2 Hz, 1H), 8.90 (d, J=2 Hz, 1H), 8.63 (d, J=8 Hz, 1H), 4.02-4.14 (m, 1H), 3.88 (s, 3H), 3.60-3.68 (m, 2H), 3.06-3.18 (m, 2H), 2.58-2.68 (m, 1H), 1.88-1.96 (m, 2H), 1.66-1.80 (m, 2H), 1.28-1.36 (m, 2H), 1.20-1.26 (m, 2H). MS: m/z 385 (M+H).

### Example 32

### Racemic 2-Cyclopropyl-N-(trans-4-(2,2,2-trifluoro-1-hydroxyethyl)cyclohexyl)thiazolo[4,5-b]pyridine-6-carboxamide

[chemical structure]

To a solution of 2-cyclopropylthiazolo[4,5-b]pyridine-6-carboxylic acid (45 mg, 0.204 mmol, Intermediate 4) in DMF (1 mL) was added DIEA (0.054 mL, 0.306 mmol), followed by HATU (93 mg, 0.245 mmol). After ˜5 min, a solution of the crude racemic 1-((trans)-4-aminocyclohexyl)-2,2,2-trifluoroethan-1-ol, trifluoroacetic acid salt (170 mg, 0.382 mmol) in DMF (2 mL) was added, followed by additional DIEA (0.143 mL, 0.817 mmol). After ˜30 min, the reaction mixture was partitioned between EtOAc and water. The organic phase was washed 1× with water and 1× with brine. The combined aqueous phases were back extracted 1× with EtOAc. This EtOAc phase was washed 1× with brine. The EtOAc extracts were combined, dried over Na₂SO₄, filtered, and concentrated. The residue was purified by silica gel chromatography, eluting with 0-60% (3:1 EtOAc:EtOH):hexanes gradient. The obtained product was repurified by silica gel chromatography, eluting with 0-60% (3:1 EtOAc:EtOH):hexanes. The product was then crystallized from CH₂Cl₂:hexanes with a minimal amount of MeOH to give racemic 2-cyclopropyl-N-(trans-4-(2,2,2-trifluoro-1-hydroxyethyl)cyclohexyl)thiazolo[4,5-b]pyridine-6-carboxamide (30 mg, 0.071 mmol, 35% yield) as a white solid. ¹H NMR (400 MHz, CD3SOCD₃) δ ppm 9.00 (d, J=2 Hz, 1H), 8.88 (d, J=2 Hz, 1H), 8.51 (d, J=8 Hz, 1H), 6.14 (d, J=7 Hz, 1H), 3.68-3.84 (m, 2H), 2.64 (tt, J=8, 5 Hz, 1H), 1.83-2.02 (m, 3H), 1.68-1.80 (m, 1H), 1.51-1.64 (m, 1H), 1.17-1.47 (m, 8H). MS: m/z 400 (M+H).

### Example 33

### N-(trans-4-(2-Hydroxypropan-2-yl)cyclohexyl)-2-(pyrrolidin-1-yl)thiazolo[4,5-b]pyridine-6-carboxamide

[chemical structure]

To a solution of 2-(pyrrolidin-1-yl)thiazolo[4,5-b]pyridine-6-carboxylic acid (49 mg, 0.198 mmol, Intermediate 13) in DMF (3 mL) was added DIEA (0.173 mL, 0.990 mmol), followed by HATU (90 mg, 0.238 mmol). After ˜5 min, 2-((trans)-4-aminocyclohexyl)propan-2-ol (62 mg, 0.396 mmol) was added, followed by additional DIEA (0.104 mL, 0.594 mmol). After ˜1 h, water (˜10 mL) was added slowly to the reaction mixture. A solid precipitated out. The mixture was stirred for a few minutes, and then the solids were collected by filtration, washed sequentially with water and with hexanes, and dried under high vacuum at 60° C. to give N-(trans-4-(2-hydroxypropan-2-yl)cyclohexyl)-2-(pyrrolidin-1-yl)thiazolo[4,5-b]pyridine-6-carboxamide (72 mg, 0.176 mmol, 89% yield) as an off-white solid. ¹H NMR (400 MHz, CD₃SOCD₃) δ ppm 8.76 (d, J=2 Hz, 1H), 8.55 (d, J=2 Hz, 1H), 8.23 (d, J=8 Hz, 1H), 4.06 (s, 1H), 3.40-3.82 (m, 5H), 2.04 (br s, 4H), 1.78-1.96 (m, 4H), 1.23-1.36 (m, 2H), 1.06-1.22 (m, 3H), 1.05 (s, 6H). MS: m/z 389 (M+H).

### Example 34

### N-(trans-4-(2-Hydroxypropan-2-yl)cyclohexyl)-2-((S)-2-methylazetidin-1-yl)thiazolo[4,5-b]pyridine-6-carboxamide

[chemical structure]

To a solution of (S)-2-(2-methylazetidin-1-yl)thiazolo[4,5-b]pyridine-6-carboxylic acid (52 mg, 0.21 mmol, Intermediate 14) in DMF (3 mL) was added DIEA (0.183 mL, 1.050 mmol), followed by HATU (96 mg, 0.252 mmol). After ˜5 min, 2-((trans)-4-aminocyclohexyl)propan-2-ol (66 mg, 0.420 mmol) was added, followed by additional DIEA (0.110 mL, 0.630 mmol). After ˜1 h, water was added, and the mixture was extracted 3× with EtOAc. The combined organic phases were washed 1× with brine, dried over Na₂SO₄, filtered, and concentrated. The residue was purified by silica gel chromatography, eluting with 10-100% (3:1 EtOAc:EtOH):hexanes gradient to give N-(trans-4-(2-hydroxypropan-2-yl)cyclohexyl)-2-((S)-2-methylazetidin-1-yl)thiazolo[4,5-b]pyridine-6-carboxamide (80 mg, 0.196 mmol, 93% yield) as a white solid. ¹H NMR (400 MHz, CD₃SOCD₃) δ ppm 8.76 (d, J=2 Hz, 1H), 8.56 (d, J=2 Hz, 1H), 8.25 (d, J=8 Hz, 1H), 4.52-4.70 (m, 1H), 4.15 (td, J=9, 5 Hz, 1H), 4.06 (s, 1H), 4.02-4.10 (m, 1H), 3.61-3.82 (m, 1H), 2.55-2.67 (m, 1H), 2.07-2.19 (m, 1H), 1.87-1.96 (m, 2H), 1.78-1.87 (m, 2H), 1.52 (d, J=6 Hz, 3H), 1.23-1.37 (m, 2H), 1.06-1.22 (m, 3H), 1.05 (s, 6H). MS: m/z 389 (M+H).

### Example 35

### 2-(Cyclopropyl(methyl)amino)-N-(trans-4-(2-hydroxypropan-2-yl)cyclohexyl)thiazolo[4,5-b]pyridine-6-carboxamide

[chemical structure]

To a solution of 2-(cyclopropyl(methyl)amino)thiazolo[4,5-b]pyridine-6-carboxylic acid (62 mg, 0.25 mmol, Intermediate 15) in DMF (3 mL) was added DIEA (0.218 mL, 1.250 mmol), followed by HATU (114 mg, 0.30 mmol). After ˜5 min, 2-((trans)-4-aminocyclohexyl)propan-2-ol (59 mg, 0.375 mmol) was added, followed by additional DIEA (0.131 mL, 0.750 mmol). After ˜2 h, water (˜10 mL) was added slowly to the reaction mixture. After stirring for a few minutes, a solid precipitated out. The mixture was stirred for ˜20 min, and then the solids were collected by filtration, washed sequentially with water and with hexanes, and dried under high vacuum at 65° C. overnight to give 2-(cyclopropyl(methyl)amino)-N-(trans-4-(2-hydroxypropan-2-yl)cyclohexyl)thiazolo[4,5-b]pyridine-6-carboxamide (70 mg, 0.171 mmol, 69% yield) as a beige solid. ¹H NMR (400 MHz, CD₃SOCD₃) δ ppm 8.77 (d, J=2 Hz, 1H), 8.57 (d, J=2 Hz, 1H), 8.25 (d, J=8 Hz, 1H), 4.05 (s, 1H), 3.62-3.78 (m, 1H), 3.24 (s, 3H), 2.81-2.95 (m, 1H), 1.86-1.95 (m, 2 H), 1.77-1.86 (m, 2H), 1.22-1.36 (m, 2H), 1.05-1.21 (m, 3H), 1.04 (s, 6H), 0.85-0.98 (m, 4H). MS: m/z 389 (M+H).

### Example 36

### 2-(Dicyclopropylamino)-N-(trans-4-(2-hydroxypropan-2-yl)cyclohexyl)thiazolo[4,5-b]pyridine-6-carboxamide

[chemical structure]

To a solution of 2-(dicyclopropylamino)thiazolo[4,5-b]pyridine-6-carboxylic acid (74 mg, 0.267 mmol, Intermediate 16) in DMF (3 mL) was added DIEA (0.233 mL, 1.335 mmol), followed by HATU (122 mg, 0.320 mmol). After ˜3 min, 2-((trans)-4-aminocyclohexyl)propan-2-ol (63 mg, 0.401 mmol) was added, followed by additional DIEA (0.140 mL, 0.801 mmol). After ˜30 min, water (˜15 mL) was added slowly to the reaction mixture. The solution remained homogeneous, but after stirring for ˜15 min more, a solid crystallized out. The mixture was diluted with 10 mL of water and stirred for another 10 min. The solids were collected by filtration, washed sequentially with water and with hexanes, and dried under high vacuum at 65° C. overnight to give 2-(dicyclopropylamino)-N-(trans-4-(2-hydroxypropan-2-yl)cyclohexyl)thiazolo[4,5-b]pyridine-6-carboxamide (87 mg, 0.199 mmol, 75% yield) as a beige solid. ¹H NMR (400 MHz, CD3SOCD₃) δ ppm 8.79 (d, J=2 Hz, 1H), 8.58 (d, J=2 Hz, 1H), 8.26 (d, J=8 Hz, 1H), 4.05 (s, 1H), 3.62-3.78 (m, 1H), 2.77-2.90 (m, 2H), 1.74-1.99 (m, 4H), 1.22-1.36 (m, 2H), 1.06-1.21 (m, 3H), 1.04 (s, 6H), 0.88-0.99 (m, 8H). MS: m/z 415 (M+H).

### Example 37

### 2-(Diisopropylamino)-N-(trans-4-(2-hydroxypropan-2-yl)cyclohexyl)thiazolo[4,5-b]pyridine-6-carboxamide

[chemical structure]

To a solution of 2-(diisopropylamino)thiazolo[4,5-b]pyridine-6-carboxylic acid (27 mg, 0.098 mmol, Intermediate 17) in DMF (3 mL) was added DIEA (0.086 mL, 0.490 mmol), followed by HATU (45 mg, 0.118 mmol). After ˜5 min, 2-((trans)-4-aminocyclohexyl)propan-2-ol (23 mg, 0.147 mmol) was added, followed by additional DIEA (0.051 mL, 0.294 mmol). After ˜45 min, water (˜10 mL) was added slowly to the reaction mixture. The solution remained homogeneous, but after stirring for ˜10 min, a solid crystallized out. The mixture was diluted with ˜7 mL of water and stirred for another 10 min. The solids were collected by filtration, washed sequentially with water and with hexanes, and dried under high vacuum at 65° C. overnight to give 2-(diisopropylamino)-N-(trans-4-(2-hydroxypropan-2-yl)cyclohexyl)thiazolo[4,5-b]pyridine-6-carboxamide (31 mg, 0.070 mmol, 72% yield) as a light beige solid. ¹H NMR (400 MHz, CD3SOCD₃) δ ppm 8.75 (d, J=2 Hz, 1H), 8.52 (d, J=2 Hz, 1H), 8.23 (d, J=8 Hz, 1H), 4.06 (s, 1H), 3.90-4.03 (m, 2H), 3.70 (m, 1H), 1.77-1.97 (m, 4H), 1.40 (d, J=6 Hz, 12H), 1.23-1.35 (m, 2H), 1.06-1.23 (m, 3H), 1.04 (s, 6H). MS: m/z 419 (M+H).

### Example 38

### 2-(tert-Butyl(methyl)amino)-N-(trans-4-(2-hydroxypropan-2-yl)cyclohexyl)thiazolo[4,5-b]pyridine-6-carboxamide

[chemical structure]

To a solution of 2-(tert-butyl(methyl)amino)thiazolo[4,5-b]pyridine-6-carboxylic acid (24 mg, 0.08 mmol, Intermediate 18) in DMF (3 mL) was added DIEA (0.070 mL, 0.400 mmol), followed by HATU (37 mg, 0.096 mmol). After ˜5 min, 2-((trans)-4-aminocyclohexyl)propan-2-ol (19 mg, 0.120 mmol) was added, followed by additional DIEA (0.042 mL, 0.240 mmol). After ˜45 min, water (˜10 mL) was added slowly to the reaction mixture. The solution remained homogeneous, but after stirring for ˜10 min a solid crystallized out. The mixture was diluted with another ˜7 mL of water and stirred for another ˜10 min. The solids were collected by filtration, washed sequentially with water and with hexanes, and dried under high vacuum. The product was repurified by silica gel chromatography, eluting with 0-50% (3:1 EtOAc:EtOH):hexanes gradient, and then crystallized from CH₂Cl₂:hexanes to give 2-(tert-butyl(methyl)amino)-N-(trans-4-(2-hydroxypropan-2-yl)cyclohexyl)thiazolo[4,5-b]pyridine-6-carboxamide (12 mg, 0.028 mmol, 35% yield) as a white solid. ¹H NMR (400 MHz, CD3SOCD₃) δ ppm 8.78 (d, J=2 Hz, 1H), 8.56 (d, J=2 Hz, 1H), 8.26 (d, J=8 Hz, 1H), 4.07 (s, 1H), 3.64-3.79 (m, 1H), 3.15 (s, 3H), 1.88-1.98 (m, 2H), 1.78-1.87 (m, 2H), 1.59 (s, 9H), 1.24-1.39 (m, 2H), 1.07-1.23 (m, 3H), 1.05 (s, 6H). MS: m/z 405 (M+H).

### Example 39

### N-((trans)-4-(2-Hydroxypropan-2-yl)cyclohexyl)-2-(methylthio)thiazolo[4,5-b]pyridine-6-carboxamide

[chemical structure]

To a solution of N-((trans)-4-(2-hydroxypropan-2-yl)cyclohexyl)-2-thioxo-2,3-dihydrothiazolo[4,5-b]pyridine-6-carboxamide (30 mg, 0.085 mmol, Intermediate 19) in DMF (1 mL) was added Cs₂CO₃ (31 mg, 0.094 mmol), followed by iodomethane (6.40 μL, 0.102 mmol). After ˜20 min, water (˜6 mL) was added dropwise to the reaction mixture. A light yellow solid precipitated out. After stirring for a few min, the solids were collected by filtration, washed sequentially with water and with hexanes, and dried under high vacuum at 65° C. overnight to give N-((trans)-4-(2-hydroxypropan-2-yl)cyclohexyl)-2-(methylthio)thiazolo[4,5-b]pyridine-6-carboxamide (24 mg, 0.062 mmol, 73% yield) as a light beige solid. ¹H NMR (400 MHz, CD₃SOCD₃) δ ppm 8.97 (d, J=2 Hz, 1H), 8.86 (d, J=2 Hz, 1H), 8.47 (d, J=8 Hz, 1H), 4.05 (s, 1H), 3.64-3.78 (m, 1H), 2.83 (s, 3H), 1.87-1.97 (m, 2H), 1.78-1.87 (m, 2H), 1.23-1.37 (m, 2H), 1.05-1.23 (m, 3H), 1.03 (s, 6H). MS: m/z 366 (M+H).

### Example 40

### N-((trans)-4-(2-Hydroxypropan-2-yl)cyclohexyl)-2-methoxythiazolo[4,5-b]pyridine-6-carboxamide

[chemical structure]

This reaction was run in 2 batches (15 mg and 12 mg), as exemplified below. A solution of 2-chloro-N-((trans)-4-(2-hydroxypropan-2-yl)cyclohexyl)thiazolo[4,5-b]pyridine-6-carboxamide (15 mg, 0.042 mmol, Intermediate 20) and DIEA (0.015 mL, 0.085 mmol) in MeOH (3 mL) was heated in a sealed tube at 70° C. overnight. Upon cooling, the crude mixtures from both batches were combined and concentrated in a rotary evaporator. The residue was dissolved in EtOAc and washed 1× with 0.1N HCl. The aqueous phase was back-extracted 1× with EtOAc. The organic phases were combined, washed with satd. brine, dried over Na₂SO₄, filtered, and concentrated. The residue was purified by silica gel chromatography, eluting with 5-60% (3:1 EtOAc:EtOH):hexanes gradient to give N-((trans)-4-(2-hydroxypropan-2-yl)cyclohexyl)-2-methoxythiazolo[4,5-b]pyridine-6-carboxamide (25 mg, 0.071 mmol, 87% yield) as a white solid. ¹H NMR (400 MHz, CD₃OD) δ ppm 8.88 (d, J=2 Hz, 1H), 8.64 (d, J=2 Hz, 1H), 4.31 (s, 3H), 3.84 (tt, J=11.4 Hz, 1H), 2.04-2.13 (m, 2H), 1.96 (br d, J=11 Hz, 2H), 1.21-1.47 (m, 5H), 1.17 (s, 6H). MS: m/z 350 (M+H).

### Example 41

### Racemic 2-cyclopropyl-N-((3S,5S)-3,5-dihydroxycyclohexyl)thiazolo[4,5-b]pyridine-6-carboxamide

[chemical structure]

To a stirred solution of 2-cyclopropylthiazolo[4,5-b]pyridine-6-carboxylic acid (100 mg, 0.454 mmol, Intermediate 4) in DMF (2 mL) was added HATU (173 mg, 0.454 mmol), followed by DIEA (0.079 mL, 0.454 mmol). The reaction mixture was stirred for 15 min, and then a solution of racemic (1S,3S)-5-aminocyclohexane-1,3-diol (72 mg, 0.545 mmol, Intermediate 21) in DMF (0.5 mL) was added. After stirring at rt for 6 h, the reaction mixture was concentrated under vacuum. The residue was purified by reverse-phase chromatography, eluting with 0-100% MeCN:water with 0.1% NH₄OH. The fractions with product were concentrated under vacuum to give racemic 2-cyclopropyl-N-((3S,5S)-3,5-dihydroxycyclohexyl)thiazolo[4,5-b]pyridine-6-carboxamide (80 mg, 0.228 mmol, 50% yield) as an off-white solid. ¹H NMR (CD₃SOCD₃) δ ppm 8.99 (d, J=2 Hz, 1H), 8.88 (d, J 15=2 Hz, 1H), 8.48 (d, J=8 Hz, 1H), 4.61 (d, J=4 Hz, 1H), 4.57 (d, J=2 Hz, 1H), 4.16-4.35 (m, 1H), 4.07 (br. s., 1H), 3.76-3.95 (m, 1H), 2.63 (s, 1H), 2.04 (d, J=11 Hz, 1H), 1.88 (d, J=12 Hz, 1H), 1.79 (d, J=12 Hz, 1H), 1.16-1.48 (m, 7H). MS: m/z 334 (M+H).

### Examples 42 and 43

### (S)-2-Cyclopropyl-N-(6-(2-hydroxypropan-2-yl)spiro[3.3]heptan-2-yl)thiazolo[4,5-b]pyridine-6-carboxamide (Ex 42) and (R)-2-cyclopropyl-N-(6-(2-hydroxypropan-2-yl)spiro[3.3]heptan-2-yl)thiazolo[4,5-b]pyridine-6-carboxamide (Ex 43)

[chemical structure]

Racemic 2-cyclopropyl-N-(6-(2-hydroxypropan-2-yl)spiro[3.3]heptan-2-yl)thiazolo[4,5-b]pyridine-6-carboxamide (24 mg, 0.065 mmol, Example 15) was resolved into its two enantiomers by chiral chromatography on a Chiralpak Chiral AD column, eluting with EtOH:heptane (45:55) with 0.1% iso-propylamine. The fractions with the desired peaks were concentrated under vacuum. The absolute configuration of the two enantiomers was tentatively assigned based on the order of elution of analogous compounds with the same spiro-amine from a related chemical series. The first eluting compound was assigned as (S)-2-cyclopropyl-N-(6-(2-hydroxypropan-2-yl)spiro[3.3]heptan-2-yl)thiazolo[4,5-b]pyridine-6-carboxamide (7.6 mg, 0.019 mmol), and it was isolated as a white solid. ¹H NMR (400 MHz, CD3SOCD₃) δ ppm 9.00 (d, J=2 Hz, 1H), 8.87 (d, J=2 Hz, 1H), 8.80 (d, J=7 Hz, 1H), 4.25-4.38 (m, 1H), 4.02 (s, 1H), 2.58-2.66 (m, 1H), 2.36-2.45 (m, 1H), 2.04-2.22 (m, 3H), 1.85-2.02 (m, 4H), 1.67-1.76 (m, 1H), 1.30-1.38 (m, 2H), 1.21-1.29 (m, 2H), 0.96 (s, 3H), 0.95 (s, 3H). MS: m/z 372 (M+H). The second eluting compound was assigned as (R)-2-cyclopropyl-N-(6-(2-hydroxypropan-2-yl)spiro[3.3]heptan-2-yl)thiazolo[4,5-b]pyridine-6-carboxamide (9.6 mg, 0.025 mmol), and it was obtained as a white solid. ¹H NMR (400 MHz, CD3SOCD₃) δ ppm 8.98 (d, J=2 Hz, 1H), 8.86 (d, J=2 Hz, 1H), 8.79 (d, J=7 Hz, 1H), 4.22-4.39 (m, 1H), 4.01 (s, 1H), 2.57-2.65 (m, 1H), 2.35-2.44 (m, 1H), 2.03-2.20 (m, 3H), 1.84-2.01 (m, 4H), 1.65-1.75 (m, 1H), 1.28-1.36 (m, 2H), 1.21-1.26 (m, 2H), 0.95 (s, 3H), 0.94 (s, 3H). MS: m/z 372 (M+H).

### Example 44—Capsule Composition

An oral dosage form for administering the present invention is produced by filing a standard two piece hard gelatin capsule with the ingredients in the proportions shown in Table 1, below.

TABLE 1

| INGREDIENTS | AMOUNTS |
|---|---|
| 2-Bromo-N-(cis)-3-hydroxy-3-methylcyclobutyl)thieno[3,2- | 7 mg |
| b]pyridine-6-carboxamide (Compound of Example 3) | |
| Lactose | 53 mg |
| Talc | 16 mg |
| Magnesium Stearate | 4 mg |

### Example 45—Injectable Parenteral Composition

An injectable form for administering the present invention is produced by stirring 1.7% by weight of 2-Bromo-N-(3-(2-hydroxypropan-2-yl)bicyclo[1,1,1]pentan-1-yl)thieno[3,2-b]pyridine-6-carboxamide (Compound of Example 11) in 10% by volume propylene glycol in water.

### Example 46 Tablet Composition

The sucrose, calcium sulfate dihydrate and a H-PGDS inhibitor as shown in Table 2 below, are mixed and granulated in the proportions shown with a 10% gelatin solution. The wet granules are screened, dried, mixed with the starch, talc and stearic acid, screened and compressed into a tablet.

TABLE 2

| INGREDIENTS | AMOUNTS |
|---|---|
| 2-Cyclopropyl-N-((trans)-4- | 12 mg |
| (hydroxymethyl)cyclohexyl)thiazolo[4,5-b]pyridine-6- | |
| carboxamide (Compound of Example 20) | |
| calcium sulfate dehydrate | 30 mg |
| Sucrose | 4 mg |
| Starch | 2 mg |
| Talc | 1 mg |
| stearic acid | 0.5 mg |

### Biological Assays

### H-PGDS RapidFire™ High Throughput Mass Spectrometry Assay

The H-PGDS RapidFire™ mass spectrometric assay monitors conversion of prostaglandin H₂ (PGH₂) to prostaglandin D₂ (PGD₂) by hematopoietic prostaglandin D synthase (H-PGDS). In the assay format described here, the substrate (PGH₂) is formed in situ by the action of cyclooxygenase-2 on arachidonic acid. This first step is set up to be fast, and generates a burst of PGH₂ at ˜10 μM. The PGH₂ is then further converted to PGD₂ by the H-PGDS enzyme. The reaction is quenched with tin (II) chloride in citric acid, which converts any remaining PGH₂ to the more stable PGF_{2α}. Plates are then read on the RapidFire™ high throughput solid phase extraction system (Agilent) which incorporates a solid phase extraction step coupled to a triple quadrupole mass spectrometer (AB SCIEX). Relative levels of PGD₂ and PGF_{2α}, which acts as a surrogate for substrate, are measured and a percent conversion calculated. Inhibitors are characterized as compounds which lower the conversion of PGH₂ to PGD₂.

Expression and Purification of H-PGDS Protein

Full length human H-PGDS cDNA (Invitrogen Ultimate ORF IOH13026) was amplified by PCR with the addition of a 5′ 6-His tag and TEV protease cleavage site. The PCR product was digested with NdeI and XhoI and ligated into pET22b+ (Merck Novagen®). Expression was carried out in E. coli strain BL21 (DE3*) using auto-induction Overnight Express™ Instant TB medium (Merck Novagen®) supplemented with 1% glycerol. The culture was first grown at 37° C. and the temperature was reduced to 25° C. when OD600 reached 2.0. Cells were harvested by centrifugation after a further 18 hours. 10 g of E. coli cell pellet was suspended to a total volume of 80 mL in lysis buffer (20 mM Tris-Cl pH 7.5, 300 mM NaCl, 20 mM imidazole, 5 mM β-mercaptoethanol, 10% glycerol). 1 mg/mL protease inhibitors (Protease Inhibitor Cocktail Set III, Merck Calbiochem®) and 1 mg/mL lysozyme were added to the cell suspension. The suspension was then sonicated for 5 min (Ultrasonic Processor VCX 750, Cole-Parmer Instrument Co.) with a micro probe (50% amplitude, 10 sec on/off) and then centrifuged at 100,000 g for 90 minutes (at 4° C.). The supernatant was loaded onto a Ni-NTA HiTrap column (5 mL, GE Healthcare, pre-equilibrated in lysis buffer). The column was washed with 10 column volumes of lysis buffer and eluted with lysis buffer containing 500 mM imidazole. The pooled protein peak fractions were concentrated using a 10 kDa centrifugal filter at 3500 g and 4° C. (Amicon Ultra-15 centrifugal filter unit with Ultracel-10 membrane from Millipore). Further purification of the concentrated protein was carried out using gel filtration chromatography on a HiLoad 26/600 Superdex75 preparative grade column (GE Healthcare Life Sciences) using 50 mM Tris pH 7.5, 50 mM NaCl, 1 mM dithiothreitol, 1 mM MgCl₂. Fractions containing the protein were pooled, concentrated as described above, and stored at −80° C.

Expression and Purification of Cyclooxyqenase-2 (COX-2) Protein

The full length human COX-2 gene (accession number L15326) was amplified by PCR to generate an EcoRI-Hindi 11 fragment containing an in-frame FLAG tag. This was subcloned into pFastBac 1 (Invitrogen). The COX-2 FLAG plasmid was recombined into the baculovirus genome according to the BAC-to-BAC protocol described by Invitrogen. Transfection into Spodoptera frugiperda (Sf9) insect cells was performed using Cellfectin (Invitrogen), according to the manufacturer's protocol. Super Sf9 cells were cultured in EX420 media (SAFC Biosciences) to a density of approximately 1.5×106 cells/mL within a wave bioreactor. Recombinant virus was added at a Multiplicity of Infection (MOI) of 5 and the culture was allowed to continue for 3 days. Cells were harvested using a continuous feed centrifuge run at 2500 g at a rate of approximately 2 L/min with cooling. The resultant cell slurry was re-centrifuged in pots (2500 g, 20 min, 4° C.) and the cell paste was stored at −80° C. 342 g of cell paste was re-suspended to a final volume of 1600 mL in a buffer of 20 mM Tris-Cl pH 7.4, 150 mM NaCl, 0.1 mM EDTA, 1.3% w/v n-octyl-β-D-glucopyranoside containing 20 Complete EDTA-free Protease Inhibitor Cocktail tablets (Roche Applied Science). The suspension was sonicated in 500 mL batches for 8×5 seconds at 10 u amplitude with the medium tip of an MSE probe sonicator and subsequently incubated at 4° C. for 90 minutes with gentle stirring. The lysate was centrifuged at 12000 rpm for 45 minutes at 4° C. in a Sorvall SLA1500 rotor. The supernatant (1400 mL) was added to 420 mL of 20 mM Tris-Cl pH 7.4, 150 mM NaCl, 0.1 mM EDTA to reduce the concentration of n-octyl-β-D-glucopyranoside to 1% w/v. The diluted supernatant was incubated overnight at 4° C. on a roller with 150 mL of anti-FLAG M2 agarose affinity gel (Aldrich-Sigma) which had been pre-equilibrated with 20 mM Tris-Cl pH 7.4, 150 mM NaCl, 0.1 mM EDTA, 1% w/v n-octyl-β-D-glucopyranoside (purification buffer). The anti-Flag M2 agarose beads were pelleted by centrifugation in 500 mL conical Corning centrifuge pots at 2000 rpm for 10 min at 4° C. in a Sorvall RC3 swing-out rotor. The supernatant (unbound fraction) was discarded and the beads were re-suspended to half the original volume in purification buffer and re-centrifuged as above. The beads were then packed into a BioRad Econo Column (5 cm diameter) and washed with 1500 mL of purification buffer at 4° C. Bound proteins were eluted with 100 μg/mL triple FLAG peptide (Aldrich-Sigma) in purification buffer. Six fractions each of 0.5 column volume were collected. After each 0.5 column volume of purification buffer was added into the column the flow was held for 10 minutes before elution. Fractions containing COX-2 were pooled resulting in a protein concentration of ˜1 mg/mL. The protein was further concentrated on Vivaspin 20 centrifugal concentrators (10 kDa cut-off) to 2.4 mg/mL and then stored at −80° C.

Test Compound Plate Preparation

Test compounds were diluted to 1 mM in DMSO and a 1:3, 11 point serial dilution was performed across a 384 well HiBase plate (Greiner Bio-one). 100 nL of this dilution series was then transferred into a 384 well v-base plate (Greiner Bio-one) using an Echo™ acoustic dispenser (Labcyte Inc) to create the assay plate. 100 nL of DMSO was added to each well in columns 6 and 18 for use as control columns.

Assay Method

5 μL of an enzyme solution containing 10 nM H-PGDS enzyme, 1.1 μM COX-2 enzyme and 2 mM reduced glutathione (Sigma-Aldrich), diluted in a buffer of 50 mM Tris-Cl pH 7.4, 10 mM MgCl₂ and 0.1% Pluronic F-127 (all Sigma-Aldrich) was added to each well of the plate except column 18 using a Multidrop Combi® dispenser (Thermo Fisher Scientific). 5 μL of enzyme solution without H-PGDS was added to each well in column 18 of the assay plate to generate 100% inhibition control wells.

Immediately after the addition of enzyme solution, 2.5 μL of a co-factor solution containing 4 μM Hemin (Sigma-Aldrich) diluted in buffer of 50 mM Tris-Cl pH 7.4 and 10 mM MgCl₂ (all Sigma-Aldrich), was added to each well using a Multidrop Combi® dispenser. 2.5 μL of substrate solution containing 80 μM arachidonic acid (Sigma-Aldrich) and 1 mM sodium hydroxide (Sigma-Aldrich) diluted in HPLC grade water (Sigma-Aldrich) was then added to each well using a Multidrop Combi® dispenser, to initiate the reaction.

The assay plates were incubated at room temperature for the duration of the linear phase of the reaction (usually 1 min 30 s-2 min, this timing should be checked on a regular basis). Precisely after this time, the reaction was quenched by the addition of 30 μL of quench solution containing 32.5 mM SnCl₂ (Sigma-Aldrich) in 200 mM citric acid (adjusted to pH 3.0 with 0.1 mM NaOH solution) to all wells using a Multidrop Combi® dispenser (Thermo Fisher Scientific). The SnCl₂ was initially prepared as a suspension at an equivalent of 600 mM in HPLC water (Sigma-Aldrich) and sufficient concentrated hydrochloric acid (Sigma-Aldrich) was added in small volumes until dissolved. The assay plates were centrifuged at 1000 rpm for 5 min prior to analysis.

The assay plates were analyzed using a RapidFire™ high throughput solid phase extraction system (Agilent) coupled to a triple quadrupole mass spectrometer (AB SCIEX) to measure relative peak areas of PGF_{2α} and PGD₂ product. Peaks were integrated using the RapidFire™ integrator software before percentage conversion of substrate to PGD₂ product was calculated as shown below:

% Conversion=((PGD₂ peak area)/(PGD₂ peak area+PGF_{2α} peak area))×100.

Data were further analyzed within Activitybase software (IDBS) using a four parameter curve fit of the following form:

$$y = \frac{a - d}{1 + {( x / c )}^{b}} + d$$

where a is the minimum, b is the Hill slope, c is the IC₅₀ and d is the maximum. Data are presented as the mean pIC₅₀.

TABLE 1

| | Potency |
|---|---|
| Ex. # | Range |
| 1 | ** |
| 2 | *** |
| 3 | * |
| 4 | * |
| 5 | ** |
| 6 | *** |
| 7 | ** |
| 8 | *** |
| 9 | *** |
| 10 | *** |
| 11 | ** |
| 12 | *** |
| 13 | ** |
| 14 | *** |
| 15 | *** |
| 16 | ** |
| 17 | * |
| 18 | ** |
| 19 | ** |
| 20 | ** |
| 21 | ** |
| 22 | ** |
| 23 | ** |
| 24 | ** |
| 25 | ** |
| 26 | *** |
| 27 | ** |
| 28 | ** |
| 29 | ** |
| 30 | ** |
| 31 | ** |
| 32 | *** |
| 33 | ** |
| 34 | *** |
| 35 | ** |
| 36 | * |
| 37 | * |
| 38 | ** |
| 39 | *** |
| 40 | ** |
| 41 | ** |
| 42 | *** |
| 43 | *** |

Legend * = pIC₅₀ 5.0-5.9 ** = pIC₅₀ 6.0-7.0, *** = pIC₅₀ 7.1-8.0

In Vivo Assays for Functional Response to Muscle Injury

Under anesthesia, the right hind limb of a mouse is restrained at the knee and the foot attached to a motorized footplate/force transducer. Needle electrodes are inserted into the upper limb, either side of the sciatic nerve and a current sufficient to elicit a maximal muscle contraction is applied. Muscle tension is produced by moving the footplate to lengthen the plantarflexor muscles while the limb is under maximal stimulation. This is repeated 60 times to fatigue the muscles of the lower limb. Anesthesia, limb immobilization and limb stimulation are then repeated at regular intervals to measure maximal isometric force in the recovering limb. 7 to 9 animals are tested for each test condition.

Eccentric contraction-induced muscle fatigue in vehicle-treated male mdx mice, 7 months of age, significantly reduced (54%) maximal isometric torque 24 hours after injury and never returned to full functional restoration. In contrast, animals (PO) dosed with 0.1, 1, and 10 mg/kg QD of the compound of Example 8 beginning 10 min prior to eccentric contraction challenge exhibited an acceleration in the kinetics of recovery. See FIG. 1.

While the preferred embodiments of the invention are illustrated by the above, it is to be understood that the invention is not limited to the precise instructions herein disclosed and that the right to all modifications coming within the scope of the following claims is reserved.

1. A compound according to Formula (I) [chemical structure]
wherein:
X is absent or selected from: N, S, and O;
Y is selected from: CH, and N;
R³ is absent or selected from:
H,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl, and
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN;
R⁴ is selected from:
F,
Cl,
Br,
I,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl,
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN,
heterocycloalkyl, and
heterocycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN;
A is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S; and
R¹ and R² are independently selected from:
hydrogen,
—OS(O)₂NH₂,
—S(O)₂CH₃,
—OH,
—CN,
F,
tetrazolyl,
methyltetrazolyl,
cycloalkyl,
morpholinyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected
from: —OH, oxo, fluoro, C₁₋₄alkoxy, cycloalkyl, —S(O)₂CH₃, —S(O)₂NH₂,
and —S(O)₂N(H)C₁₋₄alkyl, —NH₂,
—N(H)C₁₋₄alkyl, —N(H)C₁₋₄alkyl where alkyl is substituted with from 1 to
5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently
selected from: —OH, oxo, fluoro, C₁₋₄alkoxy, cycloalkyl, —NH₂,
—N(H)C₁₋₄alkyl, —N(H)C₁₋₄alkyl where the alkyl is substituted with from
1 to 5 fluoro, —N(C₁₋₄alkyl)₂, —N(C₁₋₄alkyl)₂ where the alkyls are independently substituted with from 1 to 7 fluoro,
—S(O)₂CH₃, —S(O)₂NH₂, and —S(O)₂N(H)C₁₋₄alkyl, N(C₁₋₆alkyl)₂, where each alkyl is optionally substituted with from one to six
substituents independently selected from: —OH, oxo, fluoro, and
—S(O)₂CH₃,
N(H)C₁₋₆alkyl, and
N(H)C₁₋₆alkyl substituted with from one to six substituents independently
selected from: —OH, oxo, fluoro, and —S(O)₂CH₃;
provided R³ is absent when X is absent;
or a pharmaceutically acceptable salt thereof.

2. The compound of claim 1 represented by the following Formula (II): [chemical structure]
wherein:
X¹ is absent or selected from: N, S, and O;
Y¹ is selected from: CH, and N;
R¹³ is absent or selected from:
H,
C₁₋₃ alkyl,
C₁₋₃alkyl substituted with from 1 to 3 substituents independently selected from: fluoro, oxo, C₁₋₄alkoxy, —OH, and —COOH,
C₃₋₇cycloalkyl, and
C₃₋₇cycloalkyl substituted with 1 or 2 substituents independently selected from: fluoro, oxo, C₁₋₄alkoxy, —OH, —COOH, and C₁₋₃alkyl;
R¹⁴ is selected from:
F,
Cl,
Br,
I,
C₁₋₆alkyl,
C₁₋₆alkyl substituted with from 1 to 5 substituents independently selected from: fluoro, oxo, C₁₋₄alkoxy, —OH, —COOH, —NH₂, —N(H)C₁₋₄alkyl, —N(C₁₋₄alkyl)₂ and —CN,
C₃₋₇cycloalkyl,
C₃₋₇cycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN,
heterocycloalkyl, and
heterocycloalkyl substituted with 1 or 2 substituents independently selected from: fluoro, oxo, C₁₋₄alkoxy, —OH, —COOH, C₁₋₄alkyl, —NH₂, —N(H)C₁₋₄alkyl, —N(C₁₋₄alkyl)₂ and —CN;
A¹ is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S;
R¹¹ and R¹² are independently selected from:
H,
OS(O)₂NH₂,
—S(O)₂CH₃,
—OH,
—CN,
F,
tetrazolyl,
methyltetrazolyl,
cyclopropyl,
morpholinyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cyclopropyl, cyclopentyl, cyclobutyl,
—S(O)₂CH₃, —S(O)₂NH₂, —S(O)₂N(H)C₁₋₄alkyl, —NH₂, —N(H)C₁₋₄alkyl, —N(H)C₁₋₄alkyl where alkyl is substituted with from 1 to 5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently
selected from: —OH, oxo, fluoro, C₁₋₄alkoxy, cycloalkyl, —NH₂,
—N(H)C₁₋₄alkyl, —N(H)C₁₋₄alkyl where the alkyl is substituted with from
1 to 5 fluoro, —N(C₁₋₄alkyl)₂, —N(C₁₋₄alkyl)₂ where the alkyls are independently substituted with from 1 to 7 fluoro, —S(O)₂CH₃, —S(O)₂NH₂, and —S(O)₂N(H)C₁₋₄alkyl,
N(H)C₁₋₆alkyl, and
N(H)C₁₋₆alkyl substituted with from one to six substituents independently selected from: —OH, oxo, fluoro, and —S(O)₂CH₃;
provided R¹³ is absent when X¹ is absent;
or a pharmaceutically acceptable salt thereof.

3. A compound of claim 1 represented by the following Formula (III): [chemical structure]
wherein:
X² is absent or selected from: N, S, and O;
Y² is selected from: CH, and N;
R²³ is absent or selected from:
H,
—CH₃,
—CH₂CH₃,
—CH(CH₃)₂, and
cyclopropyl;
R²⁴ is selected from:
Cl,
Br,
I,
C₁₋₄ alkyl,
C₁₋₄alkyl substituted from 1 to 3 times by F,
cyclopropyl;
methylcyclopropyl,
cyclobutyl,
azetidinyl,
methylazetidinyl, and
pyrrolidinyl;
A² is selected from:
C₄₋₇cycloalkyl,
a 4-, 5-, or 6-membered heterocycloalkyl containing one or two heteroatoms independently selected from O and N,
and
a 5-10 membered heteroaryl containing one or two heteroatoms, wherein at least one heteroatom is nitrogen and the second heteroatom, if present, is selected from N and S; and
R²¹ and R²² are independently selected from:
H,
OS(O)₂NH₂,
—S(O)₂CH₃,
—OH,
—CN,
F,
tetrazolyl
methyltetrazolyl,
cyclopropyl,
morpholinyl,
tetrazolyl,
methyltetrazolyl,
azetidinyl,
azetidinyl substituted with one or two substituents independently selected from: fluoro, —OH, —CF₃, and —CH₃,
pyridinyl,
pyridinyl substituted with —CN,
oxazolyl,
oxazolyl substituted with —C(O)OCH₂CH₃,
oxazolyl substituted with —CN,
—N(H)oxazolyl,
—N(H)oxazolyl substituted with —C(O)OCH₂CH₃,
—N(H)oxazolyl substituted with —CN,
—N(H)S(O)₂CH₃,
oxo,
C₁₋₈alkyl,
C₁₋₈alkyl substituted with from one to six substituents independently selected from: —OH, oxo, fluoro, C₁₋₄alkoxy, cyclopropyl, cyclopentyl, —S(O)₂CH₃, —NH₂, —N(H)C₁₋₄alkyl, —N(H)C₁₋₄alkyl where alkyl is substituted with from 1 to 5 fluoro, —N(C₁₋₄alkyl)₂, and —N(C₁₋₄alkyl)₂ where the alkyls are independently substituted with from 1 to 7 fluoro,
C₁₋₈alkoxy,
C₁₋₈alkoxy substituted with from one to six substituents independently selected from: —OH, oxo, fluoro, C₁₋₄alkoxy, cyclopropyl, —NH₂, —N(H)C₁₋₄alkyl, —N(H)C₁₋₄alkyl where the alkyl is substituted with
from
1 to 5 fluoro, —N(C₁₋₄alkyl)₂, —N(C₁₋₄alkyl)₂ where the alkyls are independently substituted with from 1 to 7 fluoro, —S(O)₂CH₃, —S(O)₂NH₂, and —S(O)₂N(H)C₁₋₄alkyl,
N(H)C₁₋₆alkyl, and
N(H)C₁₋₆alkyl substituted with from one to six substituents independently selected from: —OH, oxo, fluoro, and —S(O)₂CH₃;
provided R²³ is absent when X² is absent;
or a pharmaceutically acceptable salt thereof.

4. A compound of claim 1 represented by the following Formula (IV): [chemical structure]
wherein:
R³⁰ is selected from: bromide, cyclopropyl, methylcyclopropyl, cyclobutyl, azetidinyl, methylazetidinyl, —NHCH(CH₃)₂, —N(CH₃)CH(CH₃)₂, —NHCH₃, —N(CH₃)₂, —CF(CH₃)₂, —C(CH₃)₃, —CH(CH₃)₂, pyrrolidinyl, —N(CH₃)cyclopropyl, —N(cyclopropyl)₂, —NCH(CH₃)₂CH(CH₃)₂, —N(CH₃)C(CH₃)₃, —SCH₃, and —OCH₃;
Y³ is selected from: CH, and N;
A³ is selected from: cyclohexyl, cyclobutyl, bicyclopentanyl, spiroheptanyl, pyrrolidinyl, tetrahydropyranyl, and piperidinyl; and
R³¹ and R³² are independently selected from: hydrogen, fluoro, —OH, —CH₃, —OCH₂CH₂OH, oxo, —CH₂OH, —C(CH₃)₂OH, —NHCH(CH₃)CHF₂, —CH(cyclopropyl)OH, —CH(OH)CH₂S(O)₂CH₃, tetrazolyl, methyltetrazolyl, difluoroazetidinyl, fluoroazetidinyl, azetidinyl and —CH(OH)CF₃;
or a pharmaceutically acceptable salt thereof.

5. A compound of claim 1 selected from:
2-Bromo-N-(trans)-4-(2-hydroxypropan-2-yl)cyclohexyl)thieno[3,2-b]pyridine-6-carboxamide;
2-Cyclopropyl-N-(trans)-4-(2-hydroxypropan-2-yl)cyclohexyl)thieno[3,2-b]pyridine-6-carboxamide;
2-Bromo-N-(cis)-3-hydroxy-3-methylcyclobutyl)thieno[3,2-b]pyridine-6-carboxamide;
2-Cyclopropyl-N-(cis)-3-hydroxy-3-methylcyclobutyl)thieno[3,2-b]pyridine-6-carboxamide;
N-(trans)-4-(2-Hydroxypropan-2-yl)cyclohexyl)-2-(isopropylamino)thiazolo[4,5-b]pyridine-6-carboxamide;
N-(trans)-4-(2-Hydroxypropan-2-yl)cyclohexyl)-2-(isopropyl(methyl)amino)thiazolo[4,5-b]pyridine-6-carboxamide;
N-(trans)-4-(2-Hydroxypropan-2-yl)cyclohexyl)-2-(methylamino)thiazolo[4,5-b]pyridine-6-carboxamide;
2-Cyclopropyl-N-(trans)-4-(2-hydroxypropan-2-yl)cyclohexyl)thiazolo[4,5-b]pyridine-6-carboxamide;
2-(Dimethylamino)-N-(trans)-4-(2-hydroxypropan-2-yl)cyclohexyl)thiazolo[4,5-b]pyridine-6-carboxamide;
N-(trans-4-(2-Hydroxypropan-2-yl)cyclohexyl)-2-((1S,2R)-2-methylcyclopropyl)thieno[3,2-b]pyridine-6-carboxamide;
N-(trans-4-(2-hydroxypropan-2-yl)cyclohexyl)-2-((1R,2S)-2-methylcyclopropyl)thieno[3,2-b]pyridine-6-carboxamide;
2-Bromo-N-(3-(2-hydroxypropan-2-yl)bicyclo[1.1.1]pentan-1-yl)thieno[3,2-b]pyridine-6-carboxamide;
2-Cyclopropyl-N-(3-(2-hydroxypropan-2-yl)bicyclo[1.1.1]pentan-1-yl)thieno[3,2-b]pyridine-6-carboxamide;
2-Cyclopropyl-N-(3-(2-hydroxypropan-2-yl)bicyclo[1.1.1]pentan-1-yl)thiazolo[4,5-b]pyridine-6-carboxamide;
2-Cyclobutyl-N-((trans)-4-(2-hydroxypropan-2-yl)cyclohexyl)thiazolo[4,5-b]pyridine-6-carboxamide;
2-cyclopropyl-N-(6-(2-hydroxypropan-2-yl)spiro[3.3]heptan-2-yl)thiazolo[4,5-b]pyridine-6-carboxamide;
2-Cyclopropyl-N-((trans)-4-hydroxycyclohexyl)thiazolo[4,5-b]pyridine-6-carboxamide;
2-Cyclopropyl-N-((trans)-4-hydroxy-4-methylcyclohexyl)thiazolo[4,5-b]pyridine-6-carboxamide;
2-Cyclopropyl-N-((trans)-4-(2-hydroxyethoxy)cyclohexyl)thiazolo[4,5-b]pyridine-6-carboxamide;
(S)-2-Cyclopropyl-N-(2-oxopyrrolidin-3-yl)thiazolo[4,5-b]pyridine-6-carboxamide;
2-Cyclopropyl-N-((trans)-4-(hydroxymethyl)cyclohexyl)thiazolo[4,5-b]pyridine-6-carboxamide;
2-Cyclopropyl-N-((trans)-4-(3,3-difluoroazetidin-1-yl)cyclohexyl)thiazolo[4,5-b]pyridine-6-carboxamide;
2-Cyclopropyl-N-((trans)-3-(2-hydroxypropan-2-yl)cyclobutyl)thiazolo[4,5-b]pyridine-6-carboxamide;
2-Cyclopropyl-N-(trans-4-((1,1-difluoropropan-2-yl)amino)cyclohexyl)thiazolo[4,5-b]pyridine-6-carboxamide;
2-Cyclopropyl-N-((3R,6S)-6-(2-hydroxypropan-2-yl)tetrahydro-2H-pyran-3-yl)thiazolo[4,5-b]pyridine-6-carboxamide;
2-Cyclopropyl-N-((3S,6R)-6-(2-hydroxypropan-2-yl)tetrahydro-2H-pyran-3-yl)thiazolo[4,5-b]pyridine-6-carboxamide;
2-(2-Fluoropropan-2-yl)-N-(trans-4-(2-hydroxypropan-2-yl)cyclohexyl)thiazolo[4,5-b]pyridine-6-carboxamide;
2-Cyclopropyl-N-(trans-4-(cyclopropyl(hydroxy)methyl)cyclohexyl)thiazolo[4,5-b]pyridine-6-carboxamide;
2-(tert-Butyl)-N-(trans-4-(2-hydroxypropan-2-yl)cyclohexyl)thiazolo[4,5-b]pyridine-6-carboxamide;
2-cyclopropyl-N-(trans-4-(1-hydroxy-2-(methylsulfonyl)ethyl)cyclohexyl)thiazolo[4,5-b]pyridine-6-carboxamide;
2-(Azetidin-1-yl)-N-(trans-4-(2-hydroxypropan-2-yl)cyclohexyl)thiazolo[4,5-b]pyridine-6-carboxamide;
N-(trans-4-(2-Hydroxypropan-2-yl)cyclohexyl)-2-isopropylthiazolo[4,5-b]pyridine-6-carboxamide;
2-Cyclopropyl-N-(1-(1-methyl-1H-tetrazol-5-yl)piperidin-4-yl)thiazolo[4,5-b]pyridine-6-carboxamide;
2-Cyclopropyl-N-(trans-4-(2,2,2-trifluoro-1-hydroxyethyl)cyclohexyl)thiazolo[4,5-b]pyridine-6-carboxamide;
N-(trans-4-(2-Hydroxypropan-2-yl)cyclohexyl)-2-(pyrrolidin-1-yl)thiazolo[4,5-b]pyridine-6-carboxamide;
N-(trans-4-(2-Hydroxypropan-2-yl)cyclohexyl)-2-((S)-2-methylazetidin-1-yl)thiazolo[4,5-b]pyridine-6-carboxamide;
2-(Cyclopropyl(methyl)amino)-N-(trans-4-(2-hydroxypropan-2-yl)cyclohexyl)thiazolo[4,5-b]pyridine-6-carboxamide;
2-(Dicyclopropylamino)-N-(trans-4-(2-hydroxypropan-2-yl)cyclohexyl)thiazolo[4,5-b]pyridine-6-carboxamide;
2-(Diisopropylamino)-N-(trans-4-(2-hydroxypropan-2-yl)cyclohexyl)thiazolo[4,5-b]pyridine-6-carboxamide;
2-(tert-Butyl(methyl)amino)-N-(trans-4-(2-hydroxypropan-2-yl)cyclohexyl)thiazolo[4,5-b]pyridine-6-carboxamide;
N-((trans)-4-(2-Hydroxypropan-2-yl)cyclohexyl)-2-(methylthio)thiazolo[4,5-b]pyridine-6-carboxamide;
N-((trans)-4-(2-Hydroxypropan-2-yl)cyclohexyl)-2-methoxythiazolo[4,5-b]pyridine-6-carboxamide;
2-cyclopropyl-N-((3S,5S)-3,5-dihydroxycyclohexyl)thiazolo[4,5-b]pyridine-6-carboxamide;
(S)-2-Cyclopropyl-N-(6-(2-hydroxypropan-2-yl)spiro[3.3]heptan-2-yl)thiazolo[4,5-b]pyridine-6-carboxamide; and
(R)-2-cyclopropyl-N-(6-(2-hydroxypropan-2-yl)spiro[3.3]heptan-2-yl)thiazolo[4,5-b]pyridine-6-carboxamide;
or a pharmaceutically acceptable salt thereof.

6. A compound of Formula (I) or a pharmaceutically acceptable salt thereof according to claim 1 for use in therapy.

7. A compound of Formula (I) or a pharmaceutically acceptable salt thereof according to claim 1 for use in the treatment of a condition for which a H-PGDS inhibitor is indicated.

8. A compound of Formula (I) or a pharmaceutically acceptable salt thereof according to claim 1 for use in the treatment of asthma.

9. A compound of Formula (I) or a pharmaceutically acceptable salt thereof according to claim 1 for use in the treatment of Duchenne muscular dystrophy.

10. A method for the treatment of disorders in which inhibition of H-PGDS is beneficial in a human comprising administering to the human in need thereof a therapeutically effective amount of a compound of Formula (I) or a pharmaceutically acceptable salt thereof according to claim 1.

11. A method for the treatment of allergic diseases and other inflammatory conditions such as asthma, aspirin-exacerbated respiratory disease (AERD), cough, chronic obstructive pulmonary disease (including chronic bronchitis and emphysema), bronchoconstriction, allergic rhinitis (seasonal or perennial), vasomotor rhinitis, rhinoconjunctivitis, allergic conjunctivitis, food allergy, hypersensitivity lung diseases, eosinophilic syndromes including eosinophilic asthma, eosinophilic pneumonitis, eosinophilic oesophagitis, eosinophilic granuloma, delayed-type hypersensitivity disorders, atherosclerosis, rheumatoid arthritis, pancreatitis, gastritis, inflammatory bowel disease, osteoarthritis, psoriasis, sarcoidosis, pulmonary fibrosis, respiratory distress syndrome, bronchiolitis, sinusitis, cystic fibrosis, actinic keratosis, skin dysplasia, chronic urticaria, eczema and all types of dermatitis including atopic dermatitis or contact dermatitis in a human comprising administering to the human in need thereof a therapeutically effective amount of a compound of Formula (I) or a pharmaceutically acceptable salt thereof according to claim 1.

12. A method for the treatment of asthma in a human comprising administering to the human in need thereof a therapeutically effective amount of a compound of Formula (I) or a pharmaceutically acceptable salt thereof according to claim 1.

13. A method for the treatment of Duchenne muscular dystrophy in a human comprising administering to the human in need thereof a therapeutically effective amount of a compound of Formula (I) or a pharmaceutically acceptable salt thereof according to claim 1.

14. A pharmaceutical composition comprising a compound of Formula (I) or a pharmaceutically acceptable salt thereof according to claim 1 and one or more pharmaceutically acceptable carriers or excipients.

15. A pharmaceutical composition as claimed in claim 14 for the treatment of a disorder in which inhibition of H-PGDS is beneficial.

16. A pharmaceutical composition as claimed in claim 15 for the treatment or prophylaxis of asthma.

17. A pharmaceutical composition as claimed in claim 15 for the treatment or prophylaxis of Duchenne muscular dystrophy.

18. A method for the treatment of neuromuscular-related conditions selected from: Duchenne muscular dystrophy (MD), Becker MD, congenital MD (Fukuyama), Dreifuss MD, limb girdle MD, fascioscapulohumeral MD, myotonic dystrophy type I (DM1 or Steinert's), myotonic dystrophy type II (DM2 or proximal myotonic myopathy), congenital myotonia, polymyositis, dermatomyositis, amyotrophic lateral sclerosis (ALS), muscle injury, surgery-related muscle injury, traumatic muscle injury, work-related skeletal muscle injury, overtraining-related muscle injury, muscle damage due to knee replacement, muscle damage due to anterior cruciate ligament (ACL) repair, muscle damage due to plastic surgery, muscle damage due to hip replacement surgery, muscle damage due to joint replacement surgery, muscle damage due to tendon repair surgery, muscle damage due to surgical repair of rotator cuff disease, muscle damage due to surgical repair of rotator cuff injury, muscle damage due to amputation, battlefield muscle injuries, auto accident-related muscle injuries, sports-related muscle injuries, muscle lacerations, traumatic injury due to blunt force contusions, traumatic injury due to shrapnel wounds, muscle pulls or tears, traumatic injury due to burns, acute muscle strains, chronic muscle strains, weight or force stress muscle injuries, repetitive stress muscle injuries, avulsion muscle injury, compartment syndrome, muscle injuries caused by highly repetitive motions, muscle injuries caused by forceful motions, muscle injuries caused by awkward postures, muscle injuries caused by prolonged and forceful mechanical coupling between the body and an object, muscle injuries caused by vibration, muscle injuries due to unrepaired or under-repaired muscle damage coincident with a lack of recovery or lack of an increase of physical work capacity, exercise-induced delayed onset muscle soreness (DOMS), wound healing and disuse atrophy in a human comprising administering to the human in need thereof a therapeutically effective amount of a compound of Formula (I) or a pharmaceutically acceptable salt thereof according to claim 1.

19. A pharmaceutical composition as claimed in claim 14 for the treatment of neuromuscular-related conditions selected from: Duchenne muscular dystrophy (MD), Becker MD, congenital MD (Fukuyama), Dreifuss MD, limb girdle MD, fascioscapulohumeral MD, myotonic dystrophy type I (DM1 or Steinert's), myotonic dystrophy type II (DM2 or proximal myotonic myopathy), congenital myotonia, polymyositis, dermatomyositis, amyotrophic lateral sclerosis (ALS), muscle injury, surgery-related muscle injury, traumatic muscle injury, work-related skeletal muscle injury, overtraining-related muscle injury, muscle damage due to knee replacement, muscle damage due to anterior cruciate ligament (ACL) repair, muscle damage due to plastic surgery, muscle damage due to hip replacement surgery, muscle damage due to joint replacement surgery, muscle damage due to tendon repair surgery, muscle damage due to surgical repair of rotator cuff disease, muscle damage due to surgical repair of rotator cuff injury, muscle damage due to amputation, battlefield muscle injuries, auto accident-related muscle injuries, sports-related muscle injuries, muscle lacerations, traumatic injury due to blunt force contusions, traumatic injury due to shrapnel wounds, muscle pulls or tears, traumatic injury due to burns, acute muscle strains, chronic muscle strains, weight or force stress muscle injuries, repetitive stress muscle injuries, avulsion muscle injury, compartment syndrome, muscle injuries caused by highly repetitive motions, muscle injuries caused by forceful motions, muscle injuries caused by awkward postures, muscle injuries caused by prolonged and forceful mechanical coupling between the body and an object, muscle injuries caused by vibration, muscle injuries due to unrepaired or under-repaired muscle damage coincident with a lack of recovery or lack of an increase of physical work capacity, exercise-induced delayed onset muscle soreness (DOMS), wound healing and disuse atrophy.

20. A pharmaceutical composition comprising from 0.5 to 1,000 mg of a compound or pharmaceutically acceptable salt thereof as defined in claim 1, and from 0.5 to 1,000 mg of a pharmaceutically acceptable excipient.
